(19) United States
(12) Patent Application Publication
(10) Pub. No.: US 2024/0294983 A1
(43) Pub. Date: Sep. 5, 2024

(54) PROBES FOR MEASURING MOLECULAR PROXIMITY IN A SAMPLE

(71) Applicant: California Institute of Technology, Pasadena, CA (US)

(72) Inventors: Samuel J Schulte, Pasadena, CA (US); Niles A Pierce, Pasadena, CA (US)

(21) Appl. No.: 18/592,402

(22) Filed: Feb. 29, 2024

Related U.S. Application Data
(60) Provisional application No. 63/449,543, filed on Mar. 2, 2023.
(60) Provisional application No. 63/528,262, filed on Jul. 21, 2023.

(51) Int. Cl. C12Q 1/6876; C12Q 1/6844

(52) U.S. Cl. CPC C12Q 1/6876; C12Q 1/6844; C12Q 2600/166

(57) ABSTRACT

The present application relates to hybridization chain reaction (HCR). In particular, the sensitivity of hybridization chain reaction (HCR) signal amplification is combined with two or more fractional-initiator probes and one or more proximity probes able to colocalize a full HCR initiator that will trigger HCR when the targets are in proximity to one another.

### REFERENCE TO RELATED APPLICATIONS

This application claims priority to U.S. Provisional Patent Application No. 63/449,543, filed Mar. 2, 2023, and U.S. Provisional Patent Application No. 63/528,262, filed Jul. 21, 2023, the disclosures of which are hereby incorporated by reference in their entirety.

### STATEMENT REGARDING FEDERALLY SPONSORED R&D

This invention was made with government support under Grant No. EB006192 awarded by the National Institutes of Health. The government has certain rights in the invention.

### INCORPORATION BY REFERENCE OF MATERIAL IN SEQUENCE LISTING FILE

This application incorporates the material in the XML sequence listing provided herewith, entitled CALTE.164Aseglist.xml, was created on Feb. 27, 2024, and is 9,311 bytes in size.

### BACKGROUND

### Field

The present application relates to hybridization chain reaction (HCR). In particular, the sensitivity of hybridization chain reaction (HCR) signal amplification is combined with two or more fractional-initiator probes and one or more proximity probes able to colocalize a full HCR initiator that will trigger HCR when targets are in proximity to one another.

### SUMMARY

In accordance with some implementations, compositions are provided comprising a first fractional-initiator probe comprising a first target-binding domain configured to bind directly or indirectly to a first target, a first proximity domain, and a first fractional initiator; a second fractional-initiator probe comprising a second target-binding domain configured to bind directly or indirectly to a second target, a second proximity domain, and a second fractional initiator; and a proximity probe configured to bind the first proximity domain and the second proximity domain. In accordance with some implementations, a third fractional-initiator probe can be provided, comprising a third target-binding domain configured to bind directly or indirectly to a third target, a third proximity domain, and a third fractional initiator. In some embodiments the proximity probe is further configured to bind the third proximity domain.

In accordance with some implementations, the first target is a protein, a nucleic acid, a molecule, or a combination thereof, and the second target is a protein, a nucleic acid, a molecule, or a combination thereof. In accordance with some implementations, the third target is a protein, a nucleic acid, a molecule, or a combination thereof. In accordance with some implementations, the first target and the second target are bound to each other. In accordance with some implementations, the first target, the second target, and the third target are bound to each other. In accordance with some implementations, the first target and the second target are proximal to each other. In accordance with some implementations, the first target, the second target, and the third target are proximal to each other. In accordance with some implementations, the first target and the second target are the same molecule. In accordance with some implementations, the first target, the second target, and the third target are the same molecule.

In accordance with some implementations, the proximity probe contains one or more clamp domains configured to bind to some or all of the first fractional initiator and/or the second fractional initiator if one target is not proximal. In accordance with some implementations, the proximity probe contains one or more clamp domains configured to bind to some or all of the first fractional initiator, and/or the second fractional initiator, and/or the third fractional initiator if one or more targets is not proximal.

In accordance with some implementations, the first fractional-initiator probe and/or the second fractional-initiator probe comprises an antibody, a nanobody, and/or an oligonucleotide. In accordance with some implementations, the third fractional-initiator probe comprises an antibody, a nanobody, and/or an oligonucleotide.

In accordance with some implementations, when the first fractional-initiator probe is bound to the first target and the second fractional-initiator probe is bound to the second target, and when the first and second target are bound to each other and/or are proximal, then the proximity probe can bind to the first proximity domain and the second proximity domain to colocalize a full initiator.

In accordance with some implementations, when the first fractional-initiator probe is bound to the first target and the second fractional-initiator probe is bound to the second target and when the third fractional-initiator probe is bound to the third target, and when the first, second, and third target are bound to each other and/or are proximal, then the proximity probe can bind to the first proximity domain, the second proximity domain, and the third proximity domain to colocalize a full initiator.

In accordance with some implementations, the colocalized full initiator can mediate generation of a signal directly or indirectly. In accordance with some implementations, the colocalized full initiator is able to trigger HCR signal amplification or signal amplification by another method.

In accordance with some implementations, additional fractional initiator probes beyond a third fractional-initiator probe comprising a target-binding domain configured to bind directly or indirectly to target, a proximity domain, and a fractional initiator; and the proximity probe further configured to bind the proximity domain.

In accordance with some implementations, methods are provided comprising providing a sample optionally containing a first target and/or a second target; contacting the sample with a first fractional-initiator probe comprising a first target-binding domain configured to bind directly or indirectly to the first target, a first proximity domain, and a first fractional initiator; contacting the sample with a second fractional-initiator probe comprising a second target-binding domain configured to bind directly or indirectly to the second target, a second proximity domain, and a second fractional initiator; optionally, contacting the sample with a third fractional-initiator probe comprising a third target-binding domain configured to bind directly or indirectly to a third target, a third proximity domain, and a third fractional initiator; contacting the sample with a proximity probe configured to bind the first proximity domain and the second proximity domain and optionally the third proximity domain; and contacting the sample with an HCR amplifier comprising two or more HCR hairpins.

In accordance with some implementations, methods are provided comprising incubating the first fractional-initiator probe and the second fractional-initiator probe and optionally the third fractional-initiator probe in the sample to allow for binding, optionally washing to remove unbound fractional-initiator probes, incubating the proximity probe in the sample to allow for binding, optionally washing to remove unbound proximity probes, incubating the HCR amplifier in the sample, optionally washing to remove unbound HCR hairpins, and detecting a signal. In accordance with some implementations, there is a method comprising optionally removing the signal, and optionally repeating any of the above steps to detect a signal for the same or different targets.

### BRIEF DESCRIPTION OF THE DRAWINGS

FIG. 1 depicts detection of a protein:protein interaction with proximity ligation assay (PLA).

FIG. 2 depicts detection of a protein:protein interaction with a proximity-based HCR method using a kinetic trigger mechanism.

FIG. 3 depicts detection of an RNA:protein interaction with a bridge strand.

FIGS. 4A-4C depict some embodiments of HCR RNA-FISH using initiator-labeled probes.

FIGS. 5A-5B depict some embodiments of HCR RNA-FISH using fractional-initiator probes.

FIG. 6 depicts some embodiments of detection of a target complex using fractional-initiator probes and a proximity probe, wherein the fractional-initiator probes directly bind the sample.

FIG. 7 depicts some embodiments of detection of a target complex using fractional-initiator probes and a proximity probe, wherein the fractional-initiator probes indirectly bind the sample.

FIG. 8 depicts some embodiments of detection of a target using fractional-initiator probes and a proximity probe.

FIG. 9 depicts some embodiments of detection of a first target and a second target that are not in a complex with each other but are in a complex with a third target.

FIG. 10 depicts some embodiments of detection of a first target and a second target that are not in a complex with each other.

FIG. 11 depicts some embodiments of detection of a target using fractional-initiator probes and a proximity probe, wherein that fractional-initiator probes indirectly bind to the sample.

FIG. 12 depicts some embodiments of detection of 3 targets using fractional-initiator probes and a proximity probe.

FIG. 13 depicts some embodiments of detection of 3 targets using fractional-initiator probes and two proximity probes.

FIG. 14 depicts some embodiments of detection of a protein:protein complex.

FIG. 15 depicts imaging of protein:protein target complexes in human cells.

FIG. 16 depicts imaging of a protein:protein target complex in a FFPE human breast tissue section.

FIG. 17 depicts 3-plex imaging of three protein:protein target complexes in human cells.

FIG. 18 depicts simultaneous 3-plex imaging of a protein:protein target complex, a protein target, and an RNA target in human cells.

FIG. 19A-19C depicts quantitative HCR imaging of a protein:protein target complex in human cells.

FIG. 20 depicts some embodiments of detection of a protein:protein target complex with fractional-initiator nanobody probes and a proximity probe.

FIG. 21 depicts some embodiments of detection of a protein:RNA target complex.

FIG. 22 depicts some embodiments of detection of an RNA:RNA target complex.

FIGS. 23A-23C depict some embodiments of detection of a target complex using a proximity probe that allows for colocalization of the fractional initiators when both targets are present, and that clamps the entire fractional initiator in the absence of one target.

FIGS. 24A-24C depict some embodiments of detection of a target complex using a proximity probe that allows for colocalization of the fractional initiators when both targets are present, and that clamps the 5′ end of the fractional initiator in the absence of one target.

FIGS. 25A-25C depict some embodiments of detection of a target complex using a proximity probe that allows for colocalization of the fractional initiators when both targets are present, and that clamps the 3′ end of the fractional initiator in the absence of one target.

FIGS. 26A-26C depict some embodiments of detection of a target complex using a proximity probe that allows for colocalization of the fractional initiators when both targets are present, and that clamps the 5′ and 3′ ends of the fractional initiator in the absence of one target.

FIG. 27A-27B depict some embodiments of fractional-initiator probes forming a cooperative probe junction with a target RNA or with a proximity probe.

FIGS. 28A-28N depict some embodiments of initiator-labeled probes.

FIGS. 29A-29F depict some embodiments of initiator-labeled probes.

FIGS. 30A-30D depict some embodiments of probe sets comprising one or more probe units and optionally comprising one or more helper probes.

FIGS. 31A-31E depict some embodiments of fractional-initiator probes colocalized by a target RNA.

FIGS. 32A-32D depict some embodiments of fractional-initiator probes colocalized by a target.

FIGS. 33A-33E depict some embodiments of fractional-initiator probes colocalized by a target complex.

FIGS. 34A-34C depict some embodiments of probe units comprising fractional-initiator probes.

FIG. 35 depicts some embodiments of fractional-initiator probes colocalized by a target RNA.

FIGS. 36A-36R depict some embodiments of fractional-initiator probes colocalized by a target either direction or indirectly.

FIGS. 37A-37F depict some embodiments of HCR amplifiers.

FIG. 38A-38B depict some embodiments of HCR amplification using four HCR hairpins.

FIG. 39 depicts full HCR initiator i1 formed by two fractional-initiator probes colocalized by a target. Only a part of the fractional-initiator probes is depicted.

FIG. 40 depicts some embodiments of HCR amplification using two HCR hairpins.

FIGS. 41A-41E depict some embodiments of using HCR amplification to mediate CARD signal amplification for different targets and signal probes.

FIGS. 42A-42C depict some embodiments of using HCR amplification to mediate CARD signal amplification for generic targets and signal probes.

FIGS. 43A-43B depict some embodiments of using HCR amplification with reporter-labeled or fractional-reporter-labeled HCR hairpins to mediate CARD signal amplification.

FIGS. 44A-44C depict some embodiments of detection of a target complex using two fractional-initiator probes and a proximity probe comprising one or more clamps such that the proximity probe allows for colocalization of the fractional initiators to form a full initiator when both fractional-initiator probes are bound to the proximity probe but such that the one or more clamps sequester one or more portions of a fractional initiator if only one fractional-initiator probe is bound to the proximity probe.

FIGS. 45A-45D depict strong HCR signal generation in positive samples containing a protein:protein target complex and no visible staining in negative samples lacking a protein:protein target complex.

FIGS. 46A-46B depict some embodiments of an HCR imaging method for detecting a protein:protein target complex in a sample.

FIG. 47 depicts some embodiments of a protocol for simultaneous HCR imaging of protein targets, protein:protein target complexes, and RNA targets in a sample.

FIG. 48A-48D depict some embodiments of using HCR signal amplification for detection of a target or target complex in a sample.

FIG. 49A-49D depict some embodiments of HCR immunohistochemistry and HCR protein:protein imaging using primary antibody probes with or without secondary antibody probes.

FIGS. 50A-50F depict some embodiments of simultaneous HCR IHC/RNA-ISH using initiator-labeled antibody probes for protein targets and fractional-initiator DNA probes for RNA targets.

### DETAILED DESCRIPTION

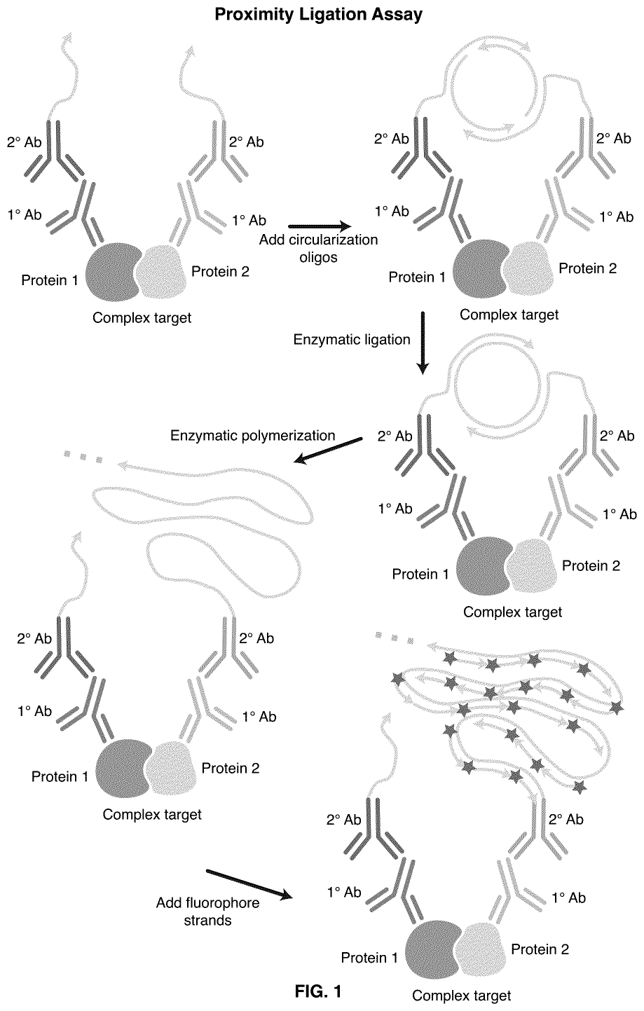

To study biological processes including replication, transcription, translation, and signaling, it is important to visualize not only molecules involved in these processes, such as RNA, protein, and DNA targets, but also complexes of these molecules. Making signal generation conditional upon the proximity of two molecules provides a sub-diffraction-limit readout in contrast to independent imaging of the same two molecules in separate channels. Target complexes have previously been imaged using proximity ligation assays (PLA) that exploit enzymatic ligation and rolling circle amplification (RCA)¹⁻¹² (for example, see FIG. 1). To detect a complex, 1° antibodies bind to the targets of interest, and 2° antibodies labeled with oligonucleotides then bind to the 1° antibodies. Two additional oligonucleotides then bind to the oligonucleotide-labeled 2° antibodies and are circularized via an enzymatic ligase step. RCA is then accomplished by an enzymatic polymerase to generate a single-stranded DNA amplification product that is detected by complementary fluorescent readout strands^{1,12} (for example, see FIG. 1). In addition to the cost and storage concerns for PLA enzymes^{1,12}, PLA suffers from false-negatives resulting from formation of non-circular ligation products¹³ and false-positives due to spurious amplification in the absence of proximal probes¹². Additionally, the amplified signal using PLA methods does not scale linearly with target abundance⁷.

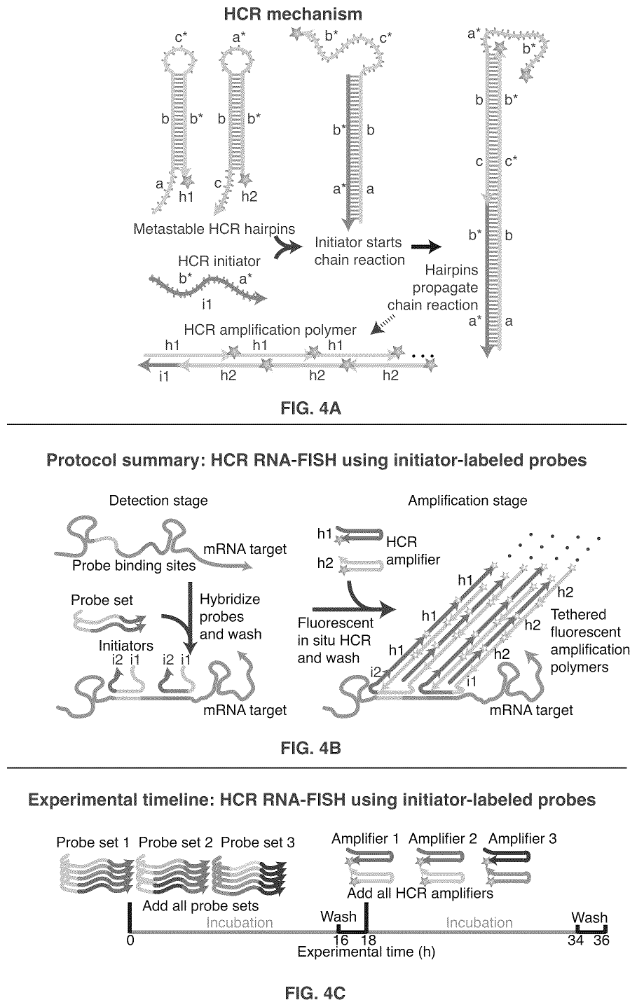

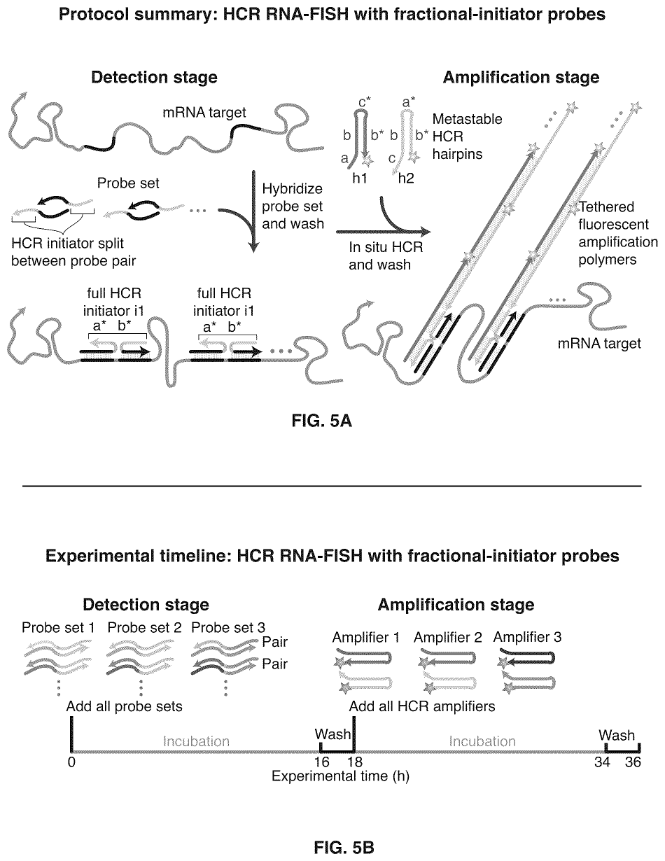

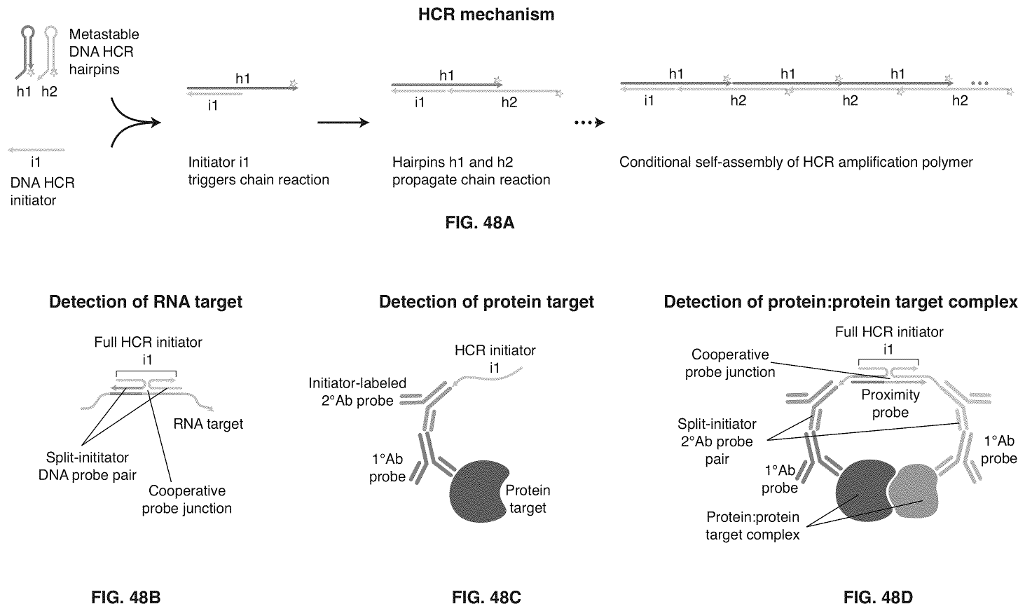

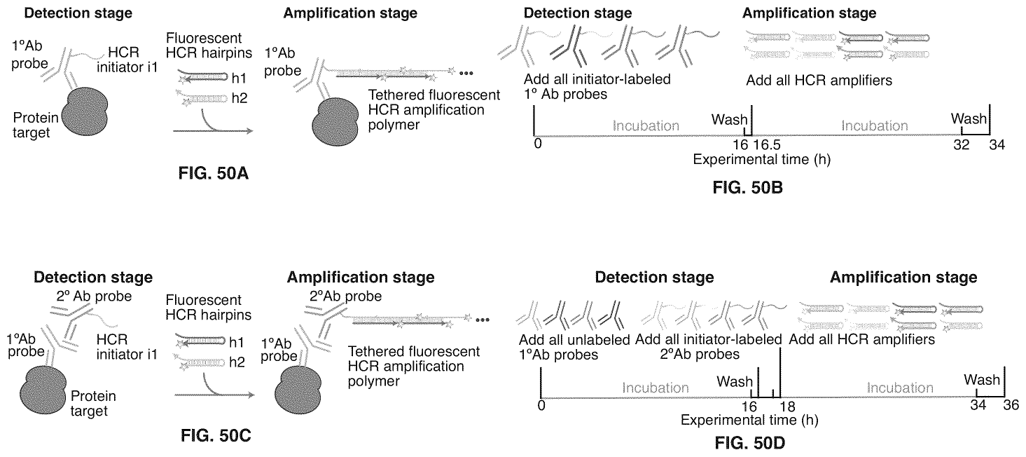

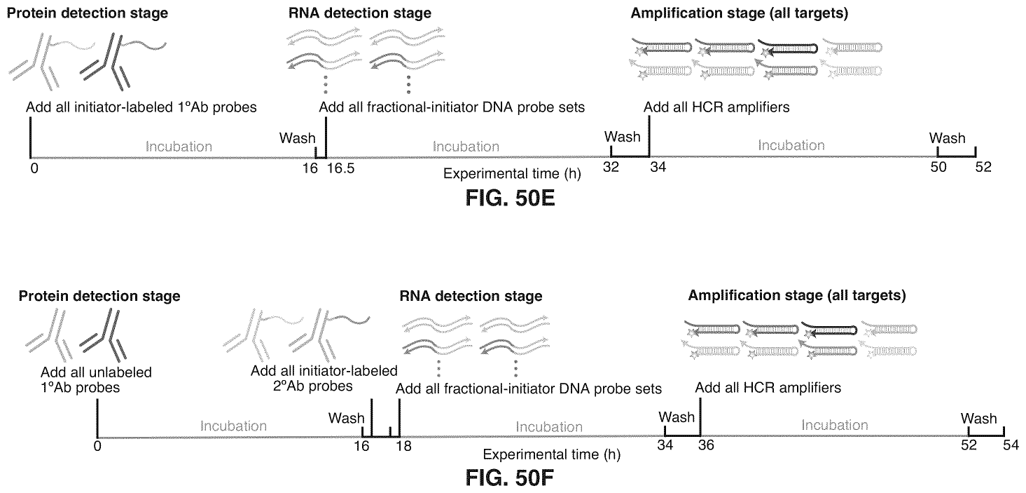

Signal amplification based on the mechanism of hybridization chain reaction (HCR)¹⁴ has been used to provide in situ signal amplification, for example for imaging RNA and protein targets within fixed biological specimens.¹⁵⁻¹⁸ In some instances, an HCR amplifier can consist of two species of kinetically trapped DNA hairpins (h1 and h2) that coexist metastably in solution, storing the energy to drive conditional self-assembly of an HCR amplification polymer upon exposure to a cognate initiator sequence (i1; for example, see FIGS. 4A, 40, and 48A).¹⁴ In some instances, using HCR RNA in situ hybridization (RNA-ISH), an RNA target can be detected using one or more pairs of fractional-initiator DNA probes, each carrying a fraction of HCR initiator i1 (FIGS. 5A and B and 48B).¹⁷ Probe pairs that hybridize specifically to proximal binding sites on the target RNA colocalize a full HCR initiator i1. In some instances, colocalization of the two (or more) fractional initiators forms a full initiator that can trigger HCR signal amplification, for example by binding to and opening one or more metastable HCR hairpin monomers. Meanwhile, any individual probes that bind nonspecifically in the sample do not colocalize the full HCR initiator i1 and do not trigger HCR, providing automatic background suppression. In some instances, using HCR immunohistochemistry (IHC), a protein target can be detected using an unlabeled primary antibody probe, which in turn is detected by a fractional-initiator secondary antibody probe that carries an HCR initiator i1 capable of triggering HCR signal amplification (FIGS. 48C, 50C, 50D, and 50F).¹⁸ In some instances, the specimen is then imaged with a fluorescence microscope to map the expression pattern of the target molecule in an anatomical context. HCR imaging protocols involve multiple stages in which different reagents are washed into or out of the sample (for example, see FIGS. 5B, 50B, 50D, 50E, and 50F).¹⁵⁻¹⁹ For example, in a detection stage, probes can be added to the fixed sample, incubated to allow probes to bind targets, and then a wash can be used to remove unused probes from the sample to reduce background resulting from non-specific probe binding; in a subsequent amplification stage, HCR amplifiers comprising fluorophore-labeled HCR hairpins can be added to the sample, incubated to allow HCR signal amplification to occur, and then a wash can be used to remove unused HCR hairpins from the sample to reduce background resulting from non-specific hairpin binding (for example, see FIGS. 5B, 50B, 50D, 50E, and 50F).

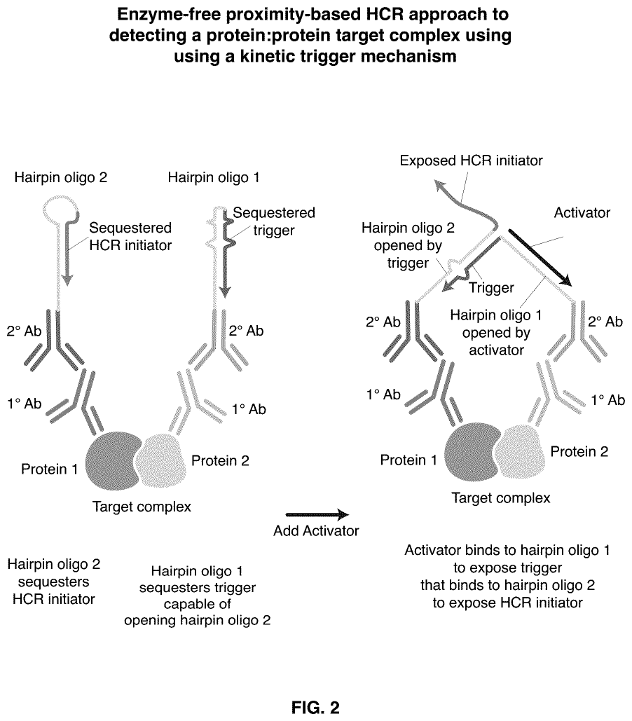

To avoid the use of enzymes for detecting a protein:protein target complex, a proximity-based HCR approach has been developed that uses a kinetic trigger mechanism to de-sequester an HCR initiator if two probes are bound to proximal target proteins^{13,20} (for example, see FIG. 2). To detect a target complex, two unlabeled primary antibodies bind to the two targets of interest, and two oligo-conjugated secondary antibody probes bind to the primary antibodies. One secondary antibody probe is conjugated to a hairpin oligo 1 and one secondary antibody probe is conjugated to a hairpin oligo 2. Hairpin oligo 1 sequesters a trigger capable of opening hairpin 2. Hairpin oligo 2 sequesters an HCR initiator. An additional activator strand is introduced, which binds to and opens hairpin oligo 1 to expose the trigger, which binds to and opens hairpin oligo 2 to expose the HCR initiator, enabling subsequent HCR signal amplification. This method has so far been limited to 1-plex applications.

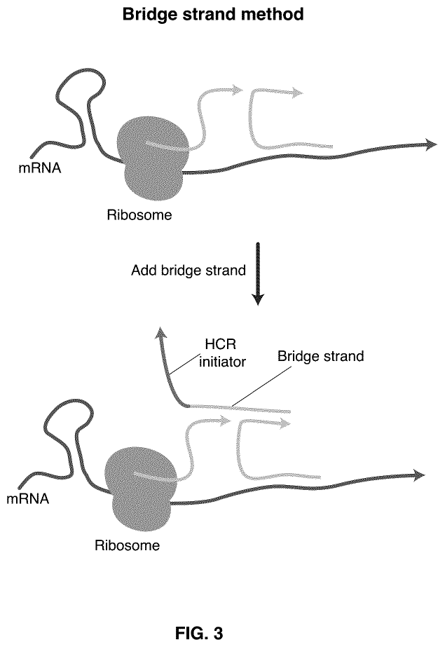

HCR signal amplification has also been used to detect ribosome interactions with mRNA²¹ using an initiator-labeled bridge strand to link one nucleic acid probe bound to the ribosome with a second nucleic acid probe bound to the an mRNA, thereby detecting a ribosomal RNA (rRNA):messenger RNA (mRNA) interaction (for example, see FIG. 3). Because the bridge strand carries a full HCR initiator, if the bridge strand binds non-specifically in the sample, amplified background will be generated. This method has so far been limited to 1-plex applications.

The systems disclosed herein address shortcomings in the existing methods, and in some embodiments enable enzyme-free, multiplexed, quantitative, high-resolution imaging of target complexes using HCR signal amplification. In some embodiments, fractional-initiator probes are utilized such that if the two or more fractional-initiator probes within a probe pair are bound to proximal targets in the sample, a further proximity probe is able to simultaneously bind proximity domains on each fractional-initiator probe so as to colocalize a full HCR initiator. In some instances, the colocalized full HCR initiator is capable of triggering HCR signal amplification by opening a metastable HCR hairpin monomer to set off a chain reaction and polymerization of HCR monomers. In some embodiments, if individual fractional-initiator probes bind to an isolated target in the sample, the proximity probe will be able to bind the isolated fractional-initiator probe, but binding of the proximity probe will not be able to colocalize a full HCR initiator, and hence will not generate an amplified signal at the site of the isolated target.

### Cooperative Probe Junctions for Proximity Measurements in a Sample

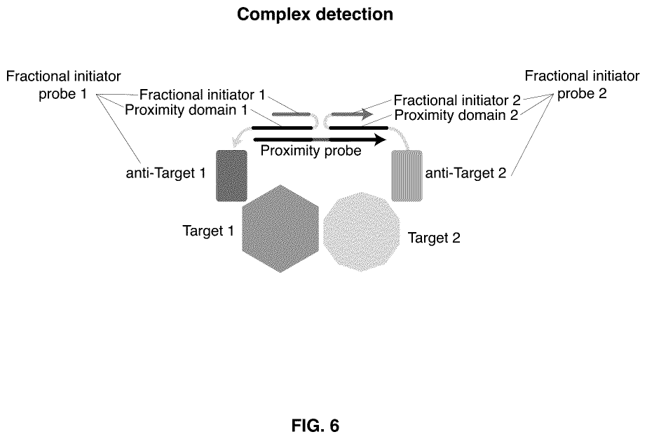

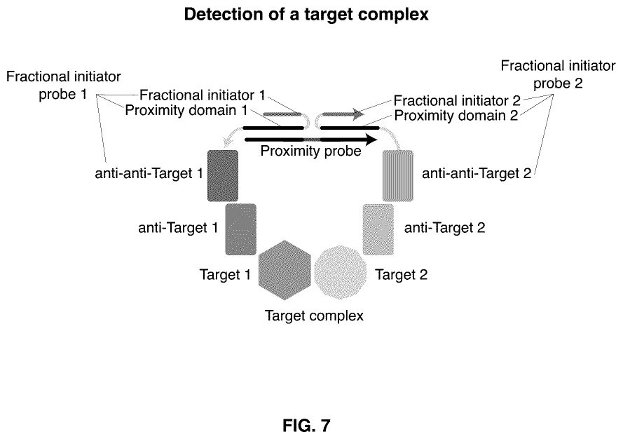

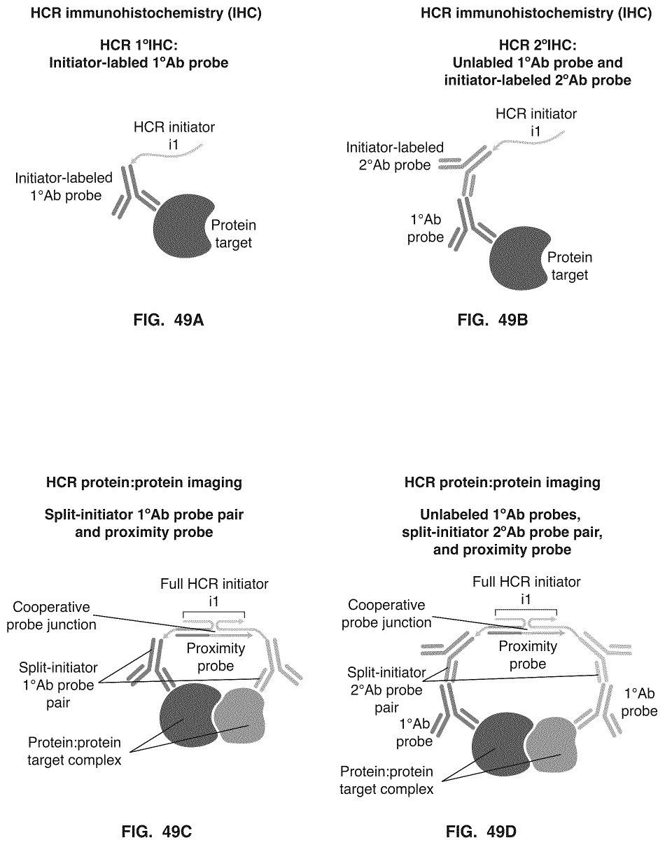

In some embodiments, a cooperative probe junction comprises two or more fractional-initiator probes that are bound by a proximity probe at their proximity domains (for example, see FIGS. 6, 7, 8, 9, 10, 11, 12, 14, 19A, 20, 21, 22, 23A, 24A, 25A, 26A, 27B, 48D, 49C, and 49D). In some embodiments, a cooperative probe junction is utilized to conduct proximity measurements within a sample. In some embodiments, a fractional-initiator probe comprises a fractional initiator (also known as an HCR fractional initiator), a proximity domain, and a target-binding domain configured to bind directly to a target (for example, see FIGS. 6, 20, 21, 22, and 49C). In some embodiments, a fractional-initiator probe comprises a fractional initiator (also known as an HCR fractional initiator), a proximity domain, and a target-binding domain configured to bind indirectly to a target (for example, see FIGS. 7, 8, 9, 10, 11, 12, 13, 14, 19A, 21, 23A, 24A, 25A, 26A, 27B, 48D, and 49D). In some embodiments, a proximity domain is a sequence within a fractional-initiator probe that is configured to be bound by a proximity probe. In some embodiments, a fractional initiator is a sequence within a fractional-initiator probe that is unable on its own to trigger HCR signal amplification, but that when colocalized with one or more fractional initiators from one or more other fractional-initiator probes via binding of the fractional-initiator probes to one or more proximity probes can form a full initiator capable of triggering HCR signal amplification.

In some embodiments: a first fractional-initiator probe comprises a first target-binding domain configured to bind directly or indirectly to a first target, a first proximity domain, and a first fractional initiator; a second fractional-initiator probe comprises a second target-binding domain configured to bind directly or indirectly to a second target, a second proximity domain, and a second fractional initiator; and a proximity probe is configured to bind the first proximity domain and the second proximity domain (for example, see FIGS. 6, 7, 9, 10, 12, 14, 19A, 20, 21, 22, 23A, 24A, 25A, 26A, 27B, 48D, 49C, and 49D). In some embodiments, binding of the proximity probe to the first proximity domain and the second proximity domain colocalizes the first fractional initiator and the second fractional initiator. In some embodiments, binding of the proximity probe to the first proximity domain and the second proximity domain colocalizes the first fractional initiator and the second fractional initiator, colocalizing a full initiator (also known as a full HCR initiator) capable of triggering HCR signal amplification.

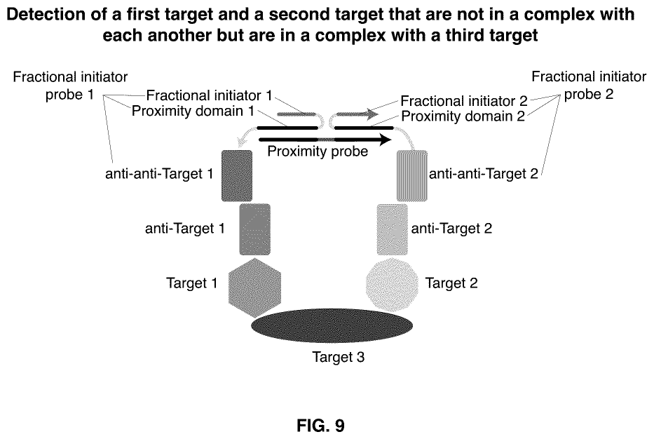

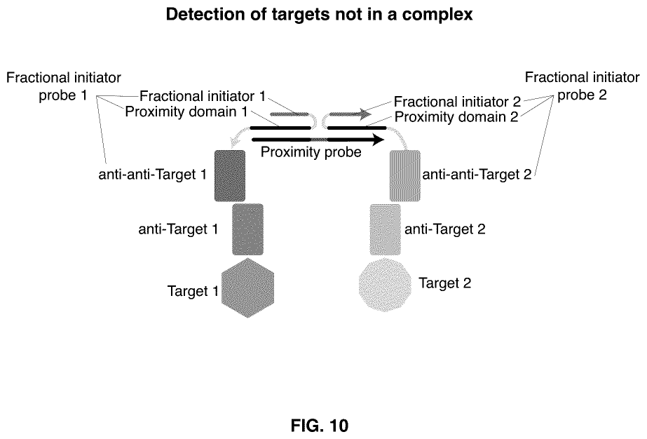

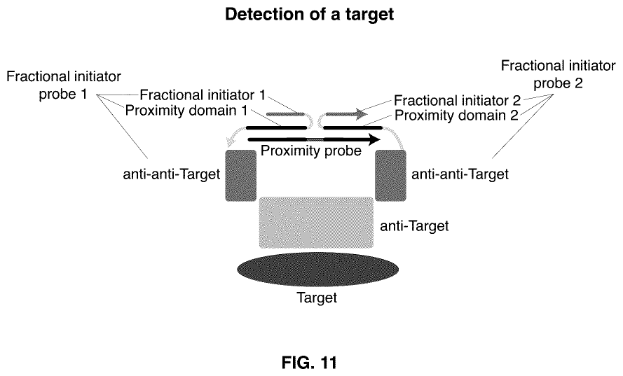

In some embodiments the first target and second target are in close enough proximity to each other that upon biding of a first fractional-initiator probe to the first target and binding of the second fractional-initiator probe to the second target, the proximity probe is able to bind to both the first proximity domain of the first fractional-initiator probe and the second proximity domain of the second fractional-initiator probe to form a cooperative probe junction. In some embodiments, the first target and the second target are bound to one another in a target complex (for example, see FIG. 7). In some embodiments, the first target and second target are not in a complex with each another but are in a complex with a third target (for example, see FIG. 9). In some embodiments, the first target and second target are proximal but are not bound to each other (for example, see FIG. 10). In some embodiments, the first target and the second target are the same molecule (for example, see FIG. 11).

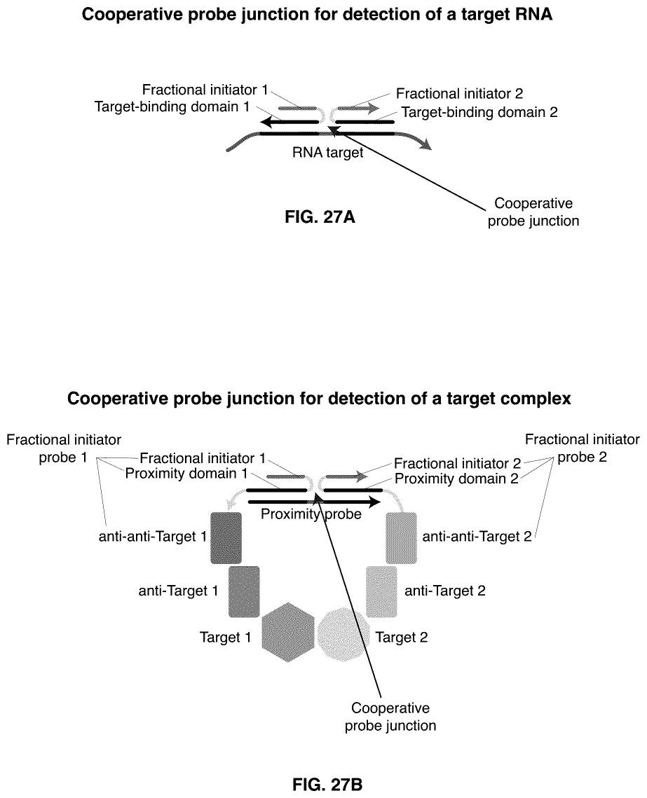

In some embodiments, the first fractional-initiator probe, the second fractional-initiator probe, and the proximity probe form a cooperative probe junction (for example, see FIGS. 27B and 48D). In some embodiments the triggering of HCR occurs as the result of the formation of the cooperative probe junction and the colocalization of the first and second fractional initiators and thus indicates that the first and second targets are proximal to each other in a sample.

The spatial resolution of three-dimensional fluorescence images is diffraction-limited to approximately 200 nm in lateral directions and 500 nm in the axial direction^{22,23}. In some embodiments, a proximity probe binds to two fractional-initiator probes to colocalize a full initiator and mediate generation of an amplified signal conditional upon the proximity of two target molecules with sub-diffraction-limit spatial resolution. In some embodiments, a proximity probe binds to two fractional-initiator probes to colocalize a full initiator and mediate generation of an amplified signal if and only if two targets are within 200 nm, or 150 nm, or 100 nm, or 90 nm, or 80 nm, or 70 nm, or 60 nm, or 50 nm, or 40 nm, or 30 nm, or 20 nm, or 10 nm, or 5 nm. In some embodiments, a proximity probe binds to two fractional-initiator probes to colocalize a full initiator and mediate generation of an amplified signal if and only if two targets are within 10 μm, or 5 μm, or 2 μm, or 1 μm, or 500 nm, or a dimension smaller than a eukaryotic cell, or a dimension smaller than a prokaryote. In some embodiments, one or more proximity probes bind to two or more fractional-initiator probes to colocalize a full initiator and mediate generation of an amplified signal conditional upon the proximity of two or more target molecules with sub-diffraction-limit spatial resolution. In some embodiments, one or more proximity probes bind to two or more fractional-initiator probes to colocalize a full initiator and mediate generation of an amplified signal if and only if two or more targets are within 200 nm, or 150 nm, or 100 nm, or 90 nm, or 80 nm, or 70 nm, or 60 nm, or 50 nm, or 40 nm, or 30 nm, or 20 nm, or 10 nm, or 5 nm. In some embodiments, one or more proximity probes bind to two or more fractional-initiator probes to colocalize a full initiator and mediate generation of an amplified signal if and only if two or more targets are within 10 μm, or 5 μm, or 2 μm, or 1 μm, or 500 nm, or a dimension smaller than a eukaryotic cell, or a dimension smaller than a prokaryote.

In some embodiments, a target complex is detected using a 3-stage protocol (for example, see FIGS. 46A and B): 1) In the detection stage, two unlabeled primary probes are provided to a sample to detect two targets. As illustrated, the primary probes may be antibody probes. Next, two secondary fractional-initiator probes (p1 and p2) are added each carrying a fraction of HCR initiator i1 and a proximity domain, wherein the secondary fractional-initiator probes are specific for the unlabeled primary probes. In some embodiments the fractional-initiator probes may comprise target binding domains configured to specifically bind the primary probes, such as the illustrated antibody target binding domains. 2) In the proximity stage, a proximity probe is provided to the sample that is able to bind to and colocalize the fractional initiator portions of the fractional-initiator probes by forming a cooperative probe junction. 3) In the amplification stage, an HCR amplifier comprising metastable labeled HCR hairpins is added to the sample such that in the presence of a colocalized full HCR initiator, the metastable labeled HCR hairpins self-assemble into a tethered labeled HCR amplification polymer. In some embodiments, the metastable labeled HCR hairpins are fluorophore-labeled. In some embodiments, in the proximity stage a cooperative probe junction is formed if the two fractional initiator probes are in close enough proximity.

In some embodiments, the use of fractional-initiator probes during the detection stage, proximity probes during the proximity stage, and metastable HCR hairpins during the amplification stage provides automatic background suppression throughout the protocol, ensuring that even if reagents bind nonspecifically in the sample, they do not generate amplified background. During the detection stage, any primary or secondary probes that bind nonspecifically in the sample do not colocalize a full HCR initiator and fail to initiate or trigger HCR on their own. In some embodiments, even if the primary and secondary fractional-initiator probes specifically bind their targets in the detection stage, the fractional initiators will fail to form a full HCR initiator and initiate HCR in the amplification stage in the absence of a proximity probe provided during the proximity stage and the formation of a cooperative probe junction. In some embodiments, even if the primary and secondary fractional-initiator probes specifically bind their targets in the detection stage, and a proximity probe is provided during the proximity stage, the fractional initiators will fail to form a full HCR initiator and initiate HCR in the amplification stage if the fractional initiators are not in close enough proximity. Likewise, during the proximity stage, any proximity probes that bind nonspecifically in the sample lack the ability to initiate HCR on their own, as the proximity probes can only mediate HCR signal amplification during the amplification stage if they bind specifically to both fractional-initiator probes to colocalize a full HCR initiator. During the amplification stage, any HCR hairpins that bind non-specifically in the sample are kinetically trapped and do not trigger formation of an HCR amplification polymer.

In some embodiments, each cooperative probe junction comprises: a fractional-initiator probe P1 comprising a target binding domain and a nucleic acid comprising a fractional initiator nucleic acid sequence and a proximity domain nucleic acid sequence; a fractional-initiator probe P2 comprising a target binding domain and a nucleic acid comprising a fractional initiator nucleic acid sequence and a proximity domain nucleic acid sequence; and a proximity probe comprising a first nucleic acid sequence complementary to the proximity domain of P1 and second nucleic acid sequence complementary to the proximity domain P2. In some embodiments, when the proximity probe binds, it brings fractional initiator sequences into proximity such that they form a full HCR initiator capable of triggering HCR signal amplification. In some embodiments, a cooperative probe junction may comprise Px additional fractional-initiator probes, where x is an integer, to detect the proximity of additional targets as the situation dictates.

In some embodiments, the cooperative probe junction colocalizes a full HCR initiator that is used to generate a signal using HCR (for example, see FIGS. 6, 7, 8, 9, 10, 11, 12, 13, 14, 19A, 20, 21, 22, 23A, 24A, 25A, 26A, 27B, 46A, 48D, 49C, and 49D). In some embodiments, the cooperative probe junction colocalizes a full HCR initiator (also known as a full initiator) that is used to generate a signal using another method that is not HCR. In some embodiments, the cooperative probe junction colocalizes a full initiator that is used to generate a signal using a complementary fluorescent strand, a branched DNA (bDNA) method, an enzymatic method, an RCA method, catalytic reporter deposition (CARD), and/or another signal generation method.

In some embodiments, the target-binding domain of a fractional-initiator probe comprises an antibody, a nanobody, a protein, a peptide, a nucleic acid, a synthetic nucleic acid analog, an aptamer, a chemically modified nucleic acid, a chemically modified protein, RNA, DNA, 2′OMe-RNA, PNA, XNA, any other material capable of base-pairing, a carbon atom, a chemical linker not capable of base-pairing, or any combination thereof. In some embodiments, the target-binding domain is capable of binding to a desired target. In some embodiments, the target-binding domain may bind directly to the target itself. In some embodiments, the target-binding domain may bind indirectly to the target, such as by binding to another molecule bound to the target, such as a primary antibody, or antibody fragment, or nanobody that is specific to the target. In some embodiments the target-biding domain binds to the target with high affinity.

In some embodiments, the target-binding domain can be selected such that it is specific for a target that is expected to be in close proximity to a second target. In some embodiments, the target may be a nucleic acid, a protein, a molecule, or a combination thereof. In some embodiments, the cooperative probe junction is able to generate signal upon detection of protein:protein complexes, RNA:protein complexes, RNA:RNA complexes, DNA:protein complexes, DNA:protein:protein complexes, or a complex of three or more RNA, DNA, and/or protein molecules in a sample.

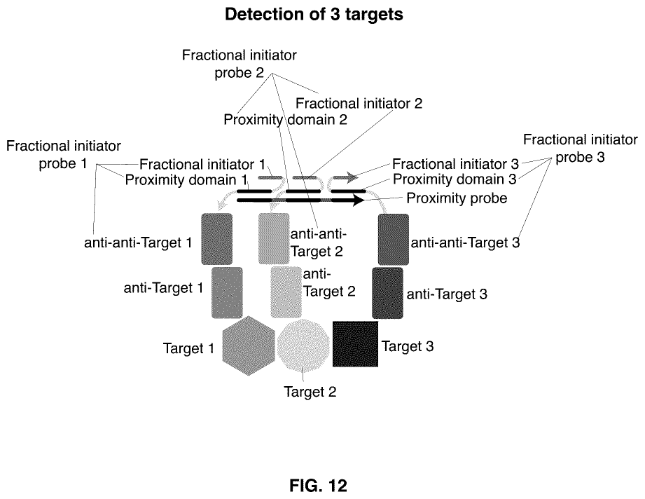

In some embodiments, fractional-initiator probes can be used to detect the proximity of three or more targets in a sample. In some embodiments, two or more fractional-initiator probes can be used to detect the proximity of two or more targets in a sample. For example, 2, 3, 4, 5, 6, 7, 8, 9, 10 . . . n fractional initiator-probes can be used in combination. In some embodiments: a first fractional-initiator probe comprises a first target-binding domain configured to bind directly or indirectly to a first target, a first proximity domain, and a first fractional initiator; a second fractional-initiator probe comprises a second target-binding domain configured to bind directly or indirectly to a second target, a second proximity domain, and a second fractional initiator; a third fractional-initiator probe comprises a third target-binding domain configured to bind directly or indirectly to a third target, a third proximity domain, and a third fractional initiator; and a proximity probe is configured to bind the first proximity domain, the second proximity domain, and the third proximity domain (for example, see FIG. 12). In some embodiments, binding of the proximity probe to the first proximity domain, the second proximity domain, and the third proximity domain colocalizes the first fractional initiator, the second fractional initiator, and the third fractional initiator. In some embodiments, binding of the proximity probe to the first proximity domain, the second proximity domain, and the third proximity domain colocalizes the first fractional initiator, the second fractional initiator, and the third fractional initiator, colocalizing a full HCR initiator capable of triggering HCR signal amplification.

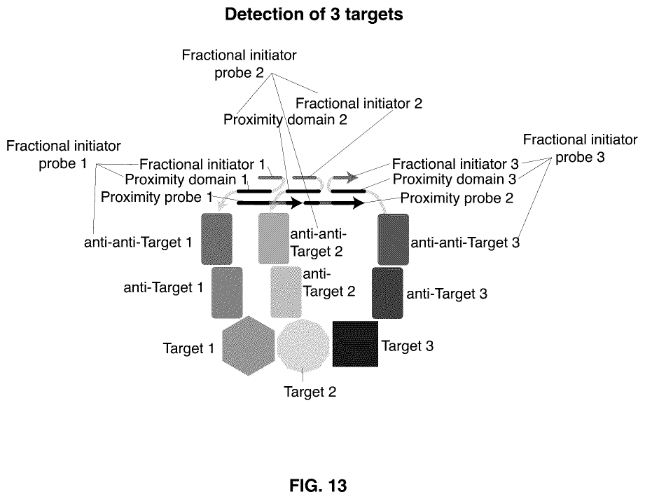

In some embodiments, two or more different proximity probes can be used in order to colocalize three or more fractional-initiator probes. In some embodiments: a first fractional-initiator probe comprises a first target-binding domain configured to bind directly or indirectly to a first target, a first proximity domain, and a first fractional initiator; a second fractional-initiator probe comprises a second target-binding domain configured to bind directly or indirectly to a second target, a second proximity domain, and a second fractional initiator; a third fractional-initiator probe comprises a third target-binding domain configured to bind directly or indirectly to a third target, a third proximity domain, and a third fractional initiator; a first proximity probe is configured to bind the first proximity domain and the second proximity domain; and a second proximity probe is configured to bind the second proximity domain and the third proximity domain (for example, see FIG. 13). In some embodiments, binding of the first proximity probe to the first proximity domain and the second proximity domain, and binding of the second proximity probe to the second proximity domain and the third proximity domain, colocalizes the first fractional initiator, the second fractional initiator, and the third fractional initiator. In some embodiments, the colocalized first fractional initiator, second fractional initiator, and third fractional initiator form a full HCR initiator capable of triggering HCR signal amplification.

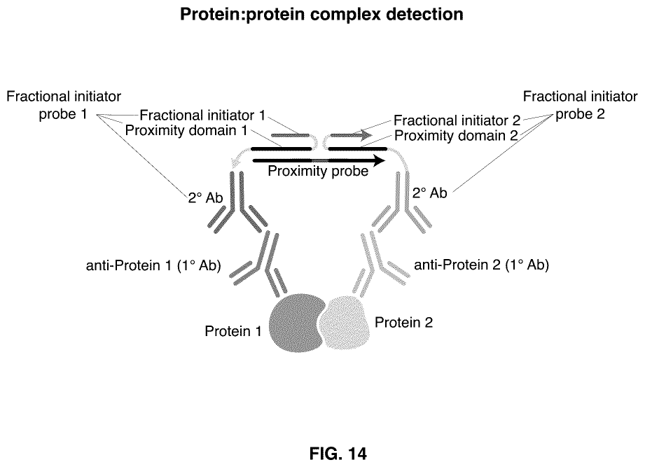

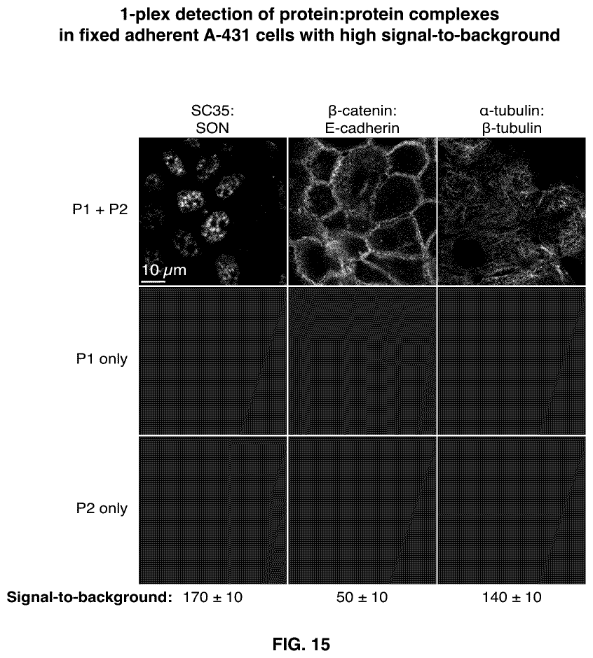

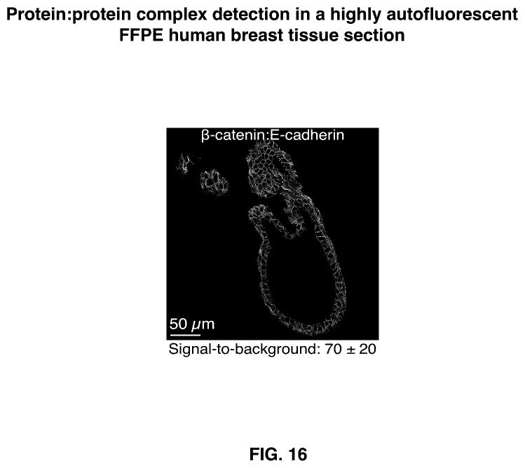

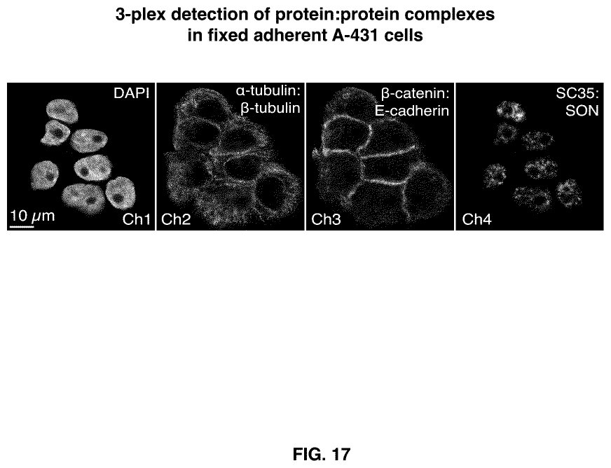

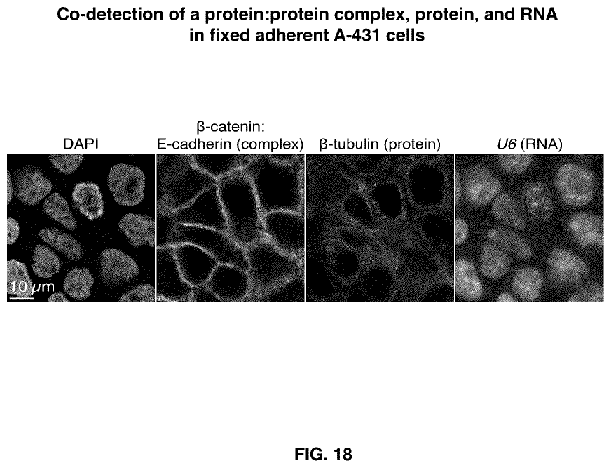

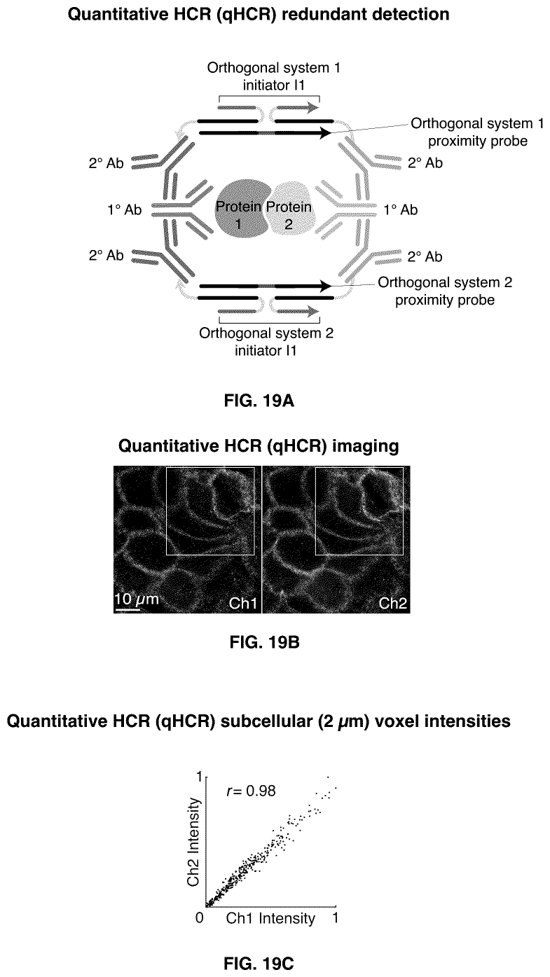

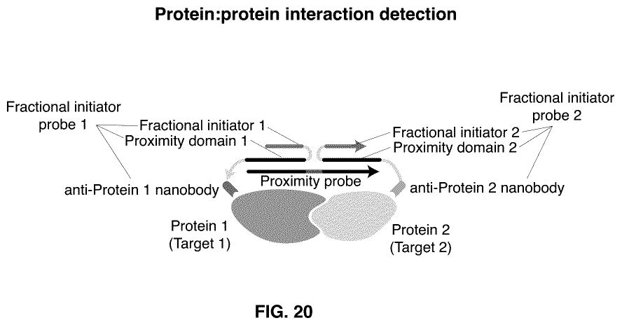

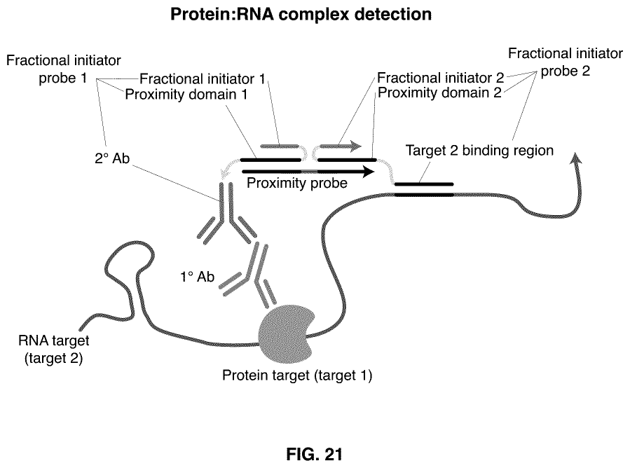

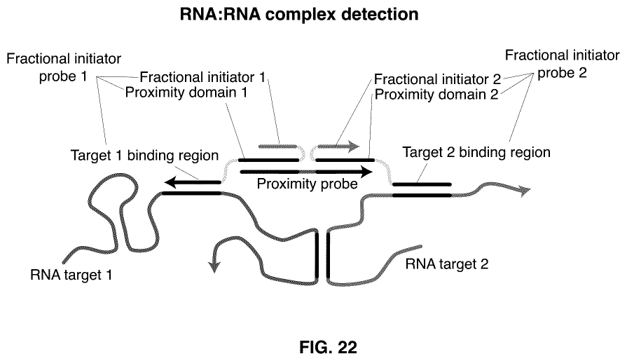

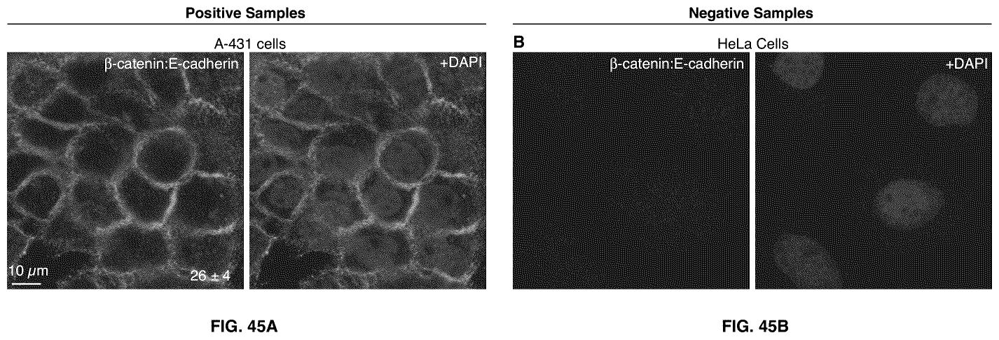

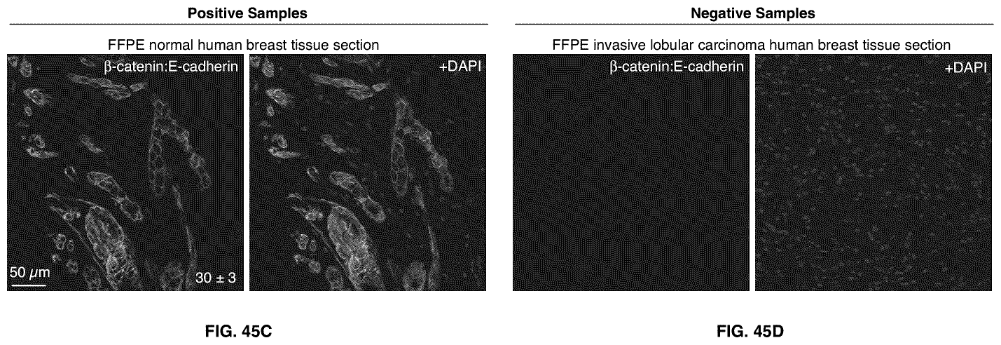

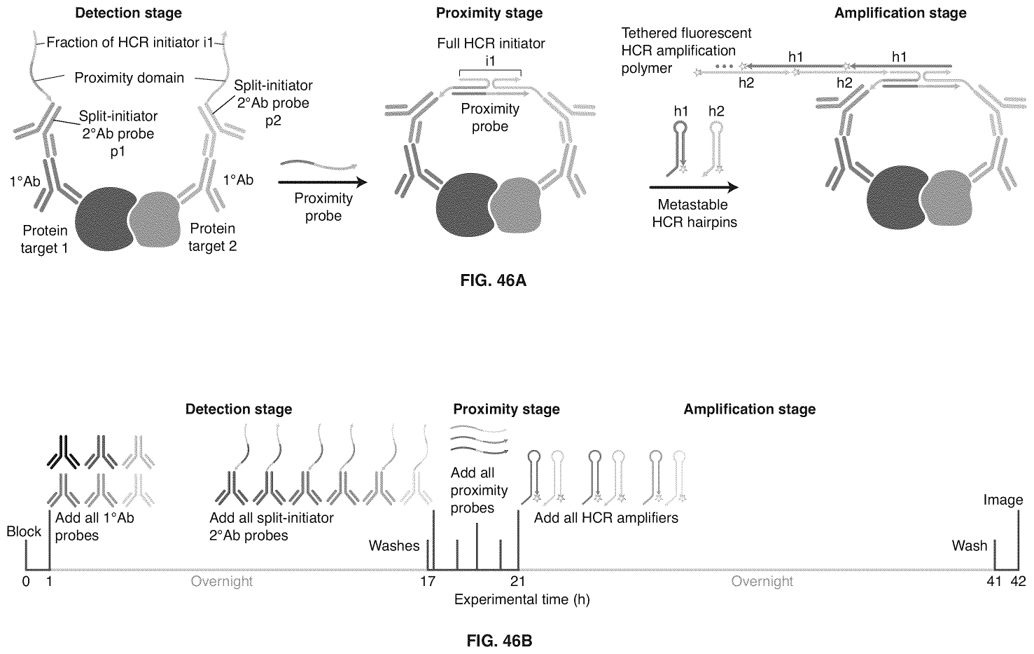

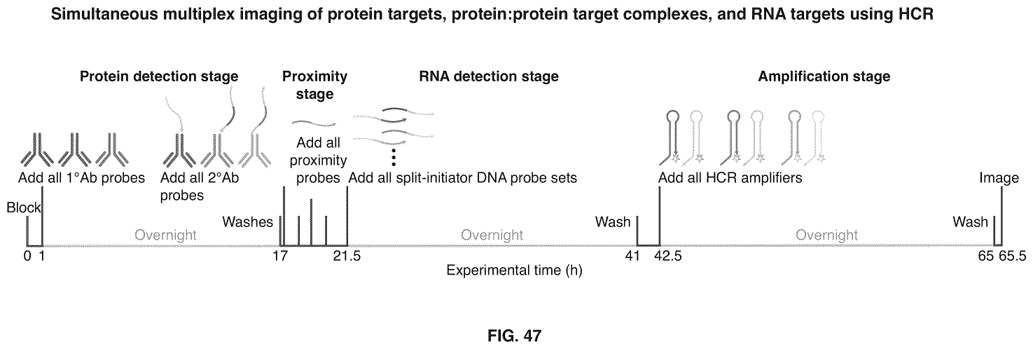

In some embodiments, the target-binding domain of a fractional-initiator probe comprises an antibody that binds directly or indirectly to a protein in the sample (for example, see FIGS. 14, 19A, 21, 46A, 48D, 49C, and 49D). In some embodiments, the target-binding domain of a fractional-initiator probe comprises a nanobody that binds directly or indirectly to a protein in the sample (for example, see FIG. 20). In some embodiments, a cooperative probe junction is used to detect a protein:protein target complex in a sample by triggering HCR in the presence of the target complex, thereby generating a detectable signal (for example, see FIGS. 14, 46A, 48D, 49C, and 49D). In some embodiments, the sample comprises a cell (for example, see FIGS. 15, 17, 18, and 19B, 45A, and 45B). In some embodiments, the sample is formalin-fixed paraffin-embedded (FFPE) tissue (for example, see FIGS. 16, 45C, and 45D). In some embodiments, one or more cooperative probe junctions are utilized to detect one or more protein:protein target complexes in a sample simultaneously (for example, see FIGS. 17 and 46B). In some embodiments, two or more cooperative probe junctions are utilized to detect two or more protein:protein target complexes in a sample simultaneously in a multiplex experiment (for example, see FIGS. 17 and 46B). In some embodiments, a cooperative probe junction comprising two or more fractional-initiator probes and one or more proximity probes is used to detect one or more target complexes simultaneous with the detection of one or more protein targets, and/or one or more RNA targets, and/or one or more DNA targets in a multiplex experiment (for example, see FIGS. 18 and 47). In some embodiments, cooperative probe junctions generate quantitative signal intensities (for example, see FIG. 19C). In some embodiments, one or more cooperative probe junctions are utilized to detect one or more RNA:protein complexes in a sample (for example, see FIG. 21). In some embodiments, one or more cooperative probe junctions are utilized to detect one or more RNA:RNA complexes in a sample (for example, see FIG. 22) or one or more DNA:protein complexes, or one or more DNA:protein:protein complexes, or a complex of three or more RNA, DNA, and/or protein molecules, and/or other molecules. In some embodiments, a cooperative probe junction comprises two or more fractional-initiator probes and one or more proximity probes. In some embodiments, a cooperative probe junction comprises three or more fractional-initiator probes and two or more proximity probes.

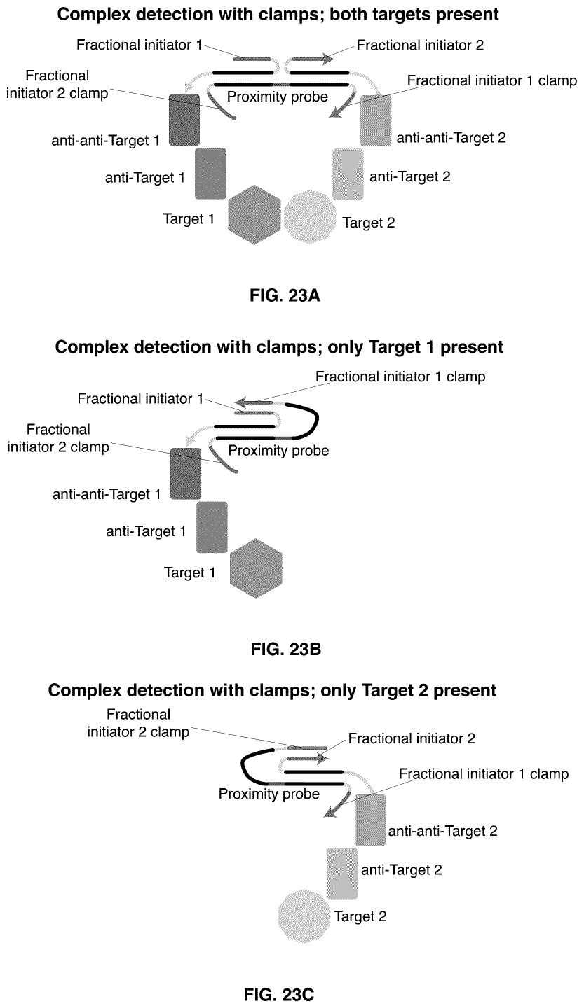

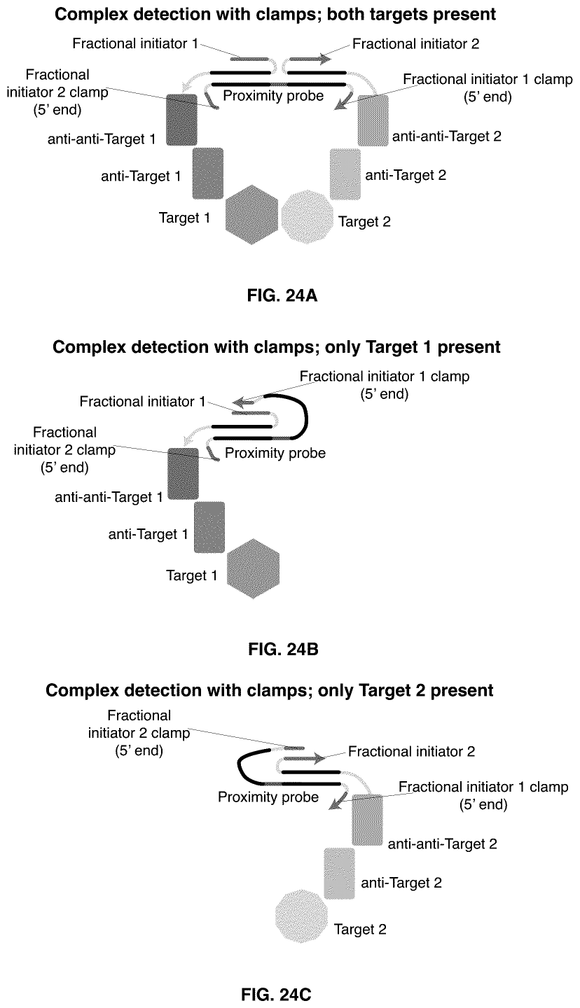

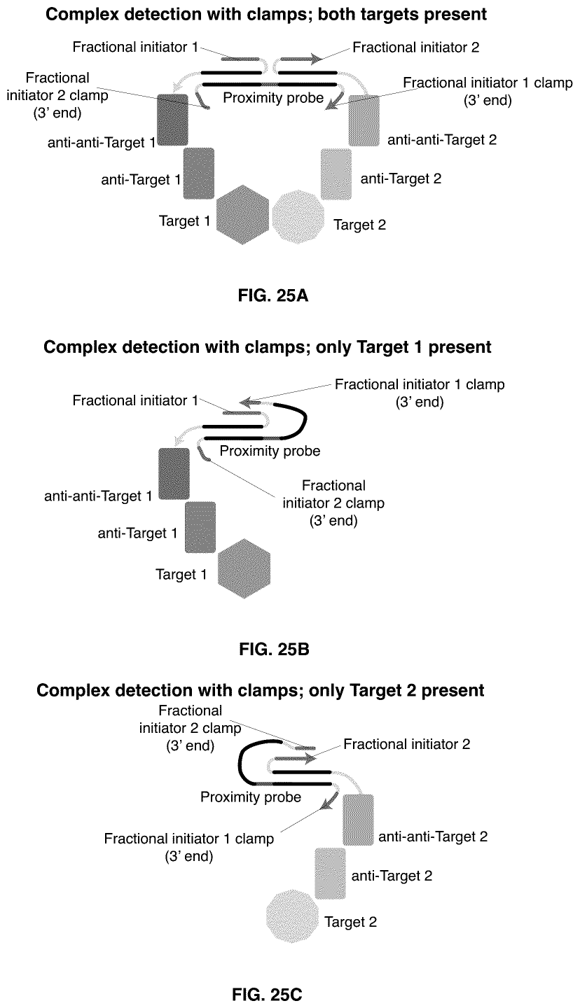

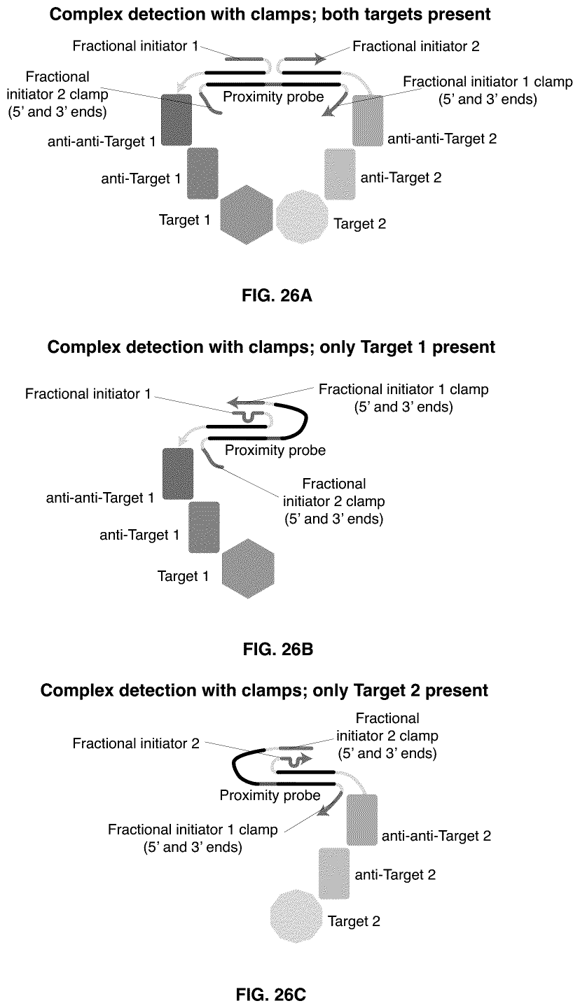

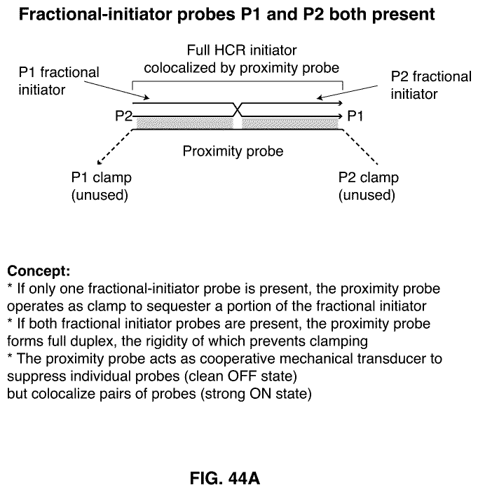

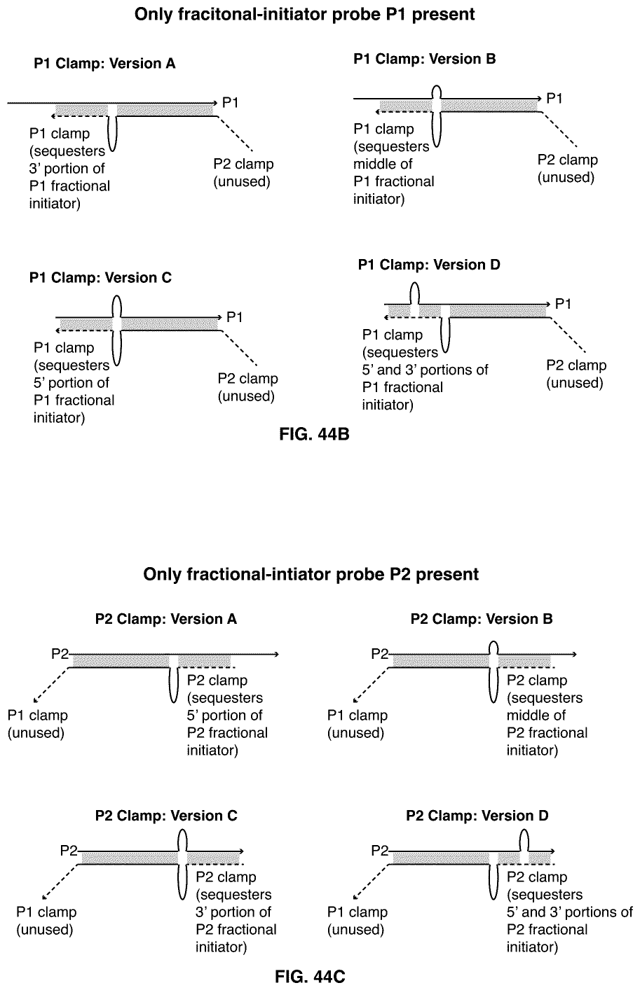

In some embodiments, a proximity probe comprises one or more clamp domains configured to bind one or more portions of a fractional initiator when only one fractional-initiator probe is bound to the proximity probe, thereby suppressing generation of amplified background (in the form of unwanted HCR signal amplification) in the absence of binding of both fractional-initiator probes to the proximity probe, and further configured such that binding of both fractional-initiator probes to the proximity probe colocalizes a full HCR initiator capable of triggering HCR signal amplification. In some embodiments, a proximity probe comprises one or more clamp domains configured to bind the entire first fractional initiator and/or second fractional initiator in the absence of one of the targets in a target complex (for example, see FIGS. 23 and 44A-44C). In some embodiments, a proximity probe comprises one or more clamp domains configured to bind the 5′ end of the first fractional initiator and/or the 5′ end of the second fractional initiator in the absence of one of the targets in a target complex (for example, see FIG. 24). In some embodiments, a proximity probe comprises one or more clamp domains configured to bind the 3′ end of the first fractional initiator and/or the 3′ end of the second fractional initiator in the absence of one of the targets in a target complex (for example, see FIG. 25). In some embodiments, a proximity probe comprises one or more clamp domains configured to bind the 5′ and 3′ ends of the first fractional initiator and/or the 5′ and 3′ ends of the second fractional initiator in the absence of one of the targets in a target complex (for example, see FIG. 26). In some embodiments, the clamp domain can be complementary to the fractional initiator domain. In some embodiments, a proximity probe does not comprise a clamp domain configured to bind to some or all of the first fractional initiator and/or the second fractional initiator in the absence of one of the targets in a target complex (for example, see FIGS. 6, 7, 8, 9, 10, 11, 12, 13, 14, 19A, 20, 21, 22).

In some embodiments, the HCR hairpin monomers (also known as HCR hairpins and HCR monomers) are able to polymerize when the proximity probe binds to all of the proximity domains in the proximal fractional initiator probes and colocalizes a full HCR initiator. In some embodiments, the HCR hairpin monomers can comprise a fluorophore, a chromophore, a luminophore, a phosphor, a FRET pair, or other labels such that the formed polymers can be detected. In some embodiments, the signal is read using a fluorescence microscope, a fluorescence scanner, a camera, a mobile phone camera, a mass spectrometer, a mass spectrometry microscope, a radioactive scanner, or another instrument suitable for detecting a signal. In some embodiments, the signal generation comprises a fluorophore, a chromophore, a luminophore, a phosphor, a FRET pair, a member of a FRET pair, a quencher, a fluorophore/quencher pair, a rare earth element or compound, a radioactive molecule, a nucleotide, an amino acid, an oligonucleotide, DNA, RNA, 2′OMe-RNA, a chemically modified nucleic acid, a synthetic nucleic acid analog, a chemically modified protein, a synthetic protein analog, a peptide, a binding substrate, a carbon atom, a chemical linker, a magnetic molecule, carbon black (CB), carbon nanotubes, magnetized carbon nanotubes, gold nanoparticles (AuNP), gold nanoshells, gold nanorods, silver-shelled gold nanoparticles, latex, magnetic nanoparticles, silica nanoparticles, a fluorophore, fluorophore-loaded nanoparticles, dye-loaded nanoparticles, an enzyme, any combination thereof, or any other molecule that facilitates measurement of a signal. In some embodiments, a hapten, a ligand, an oligonucleotide, digoxigenin (DIG), fluorescein isothiocyanate (FITC), a fluorophore, biotin, dinitrophenol, aniline, an enzyme, or another molecule or complex that can be recognized by a binding partner is utilized to facilitate generation of a signal.

### Signal Generation

In some embodiments, an initiator or a colocalized full initiator is used to mediate generation of a signal directly or indirectly. In some embodiments, an initiator or colocalized full initiator mediates signal amplification.²⁴⁻²⁶ In some embodiments, an initiator or colocalized full initiator mediates signal amplification via HCR,¹⁵⁻¹⁹ branched DNA (bDNA),²⁷⁻³³ rolling circle amplification (RCA),³⁴⁻³⁸ catalytic reporter deposition (CARD),^{26,31,39-59} polymerase chain reaction (PCR),^{30,60} proximity ligation assay (PLA),^{2-6,8-12,61,62} and/or any other signal amplification method that increases the signal intensity. In some embodiments, the initiator or colocalized full initiator is configured to bind to a readout probe comprising one or more reporters that directly or indirectly lead to generation of a signal.

### Hybridization Chain Reaction (HCR) Signal Amplification

In some embodiments, an HCR amplifier comprises two or more HCR hairpins (for example, see the HCR amplifiers of FIGS. 5A-5B, 37A-37F, and 38A-38B). In some embodiments, each HCR hairpin (also referred to as an HCR hairpin monomer or as an HCR monomer) comprises an input domain with a single-stranded toehold and a stem section, and an output domain with a single-stranded loop and a complement to the stem section (for example, see the HCR hairpins of FIGS. 5A-5B, 39, 40, 37A-37F, and 38A-38B).

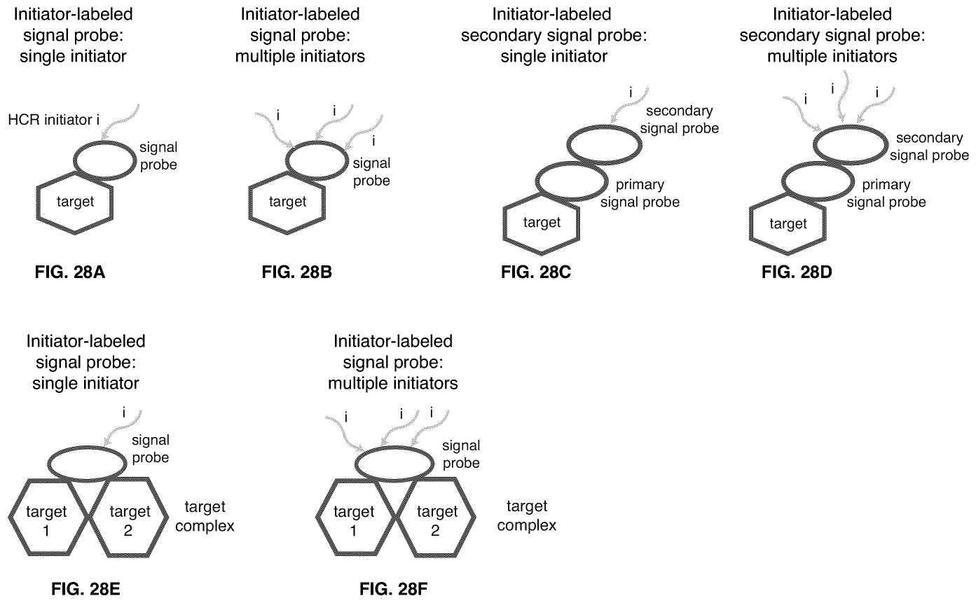

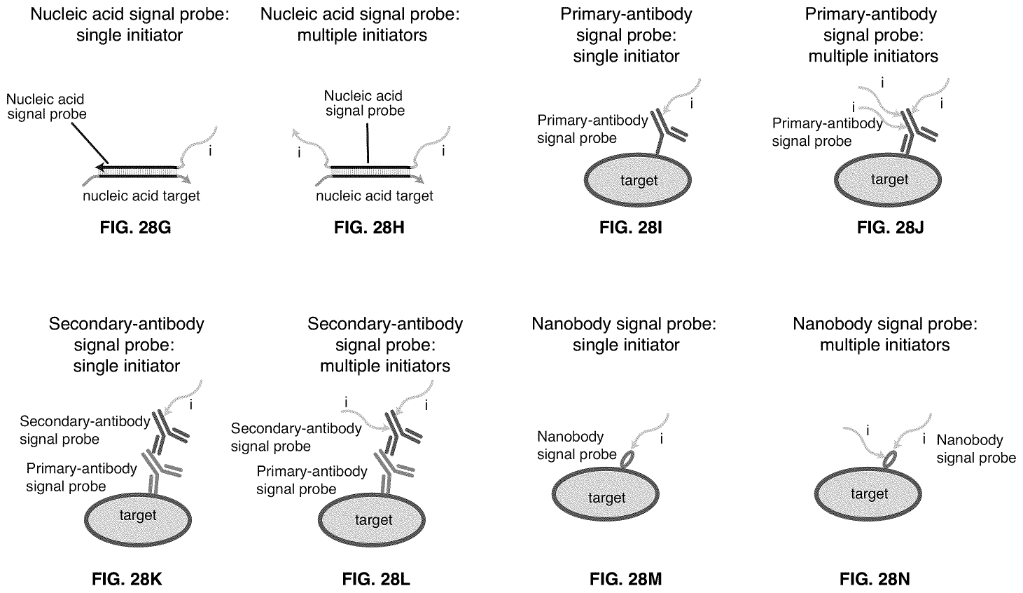

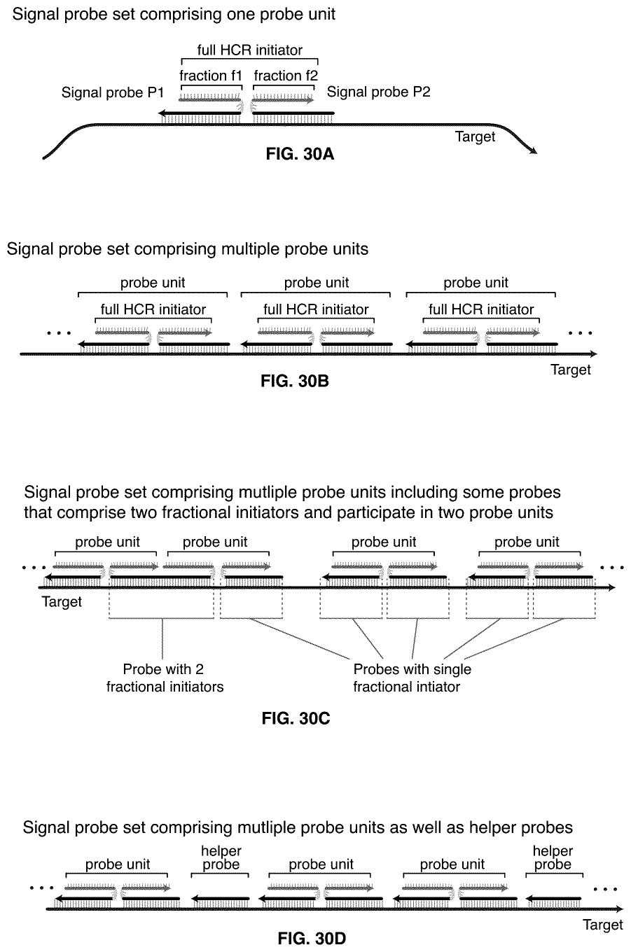

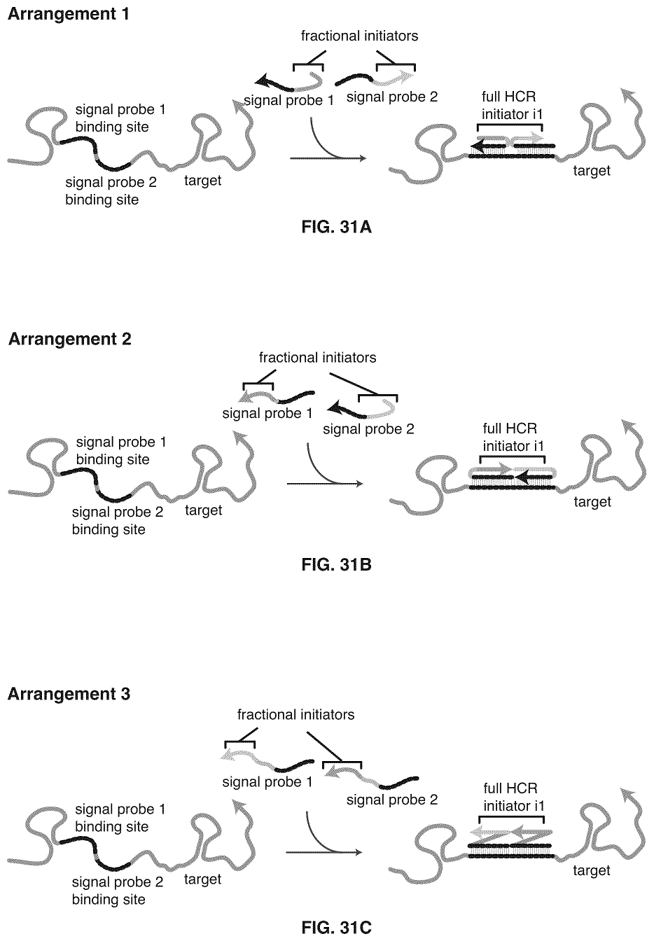

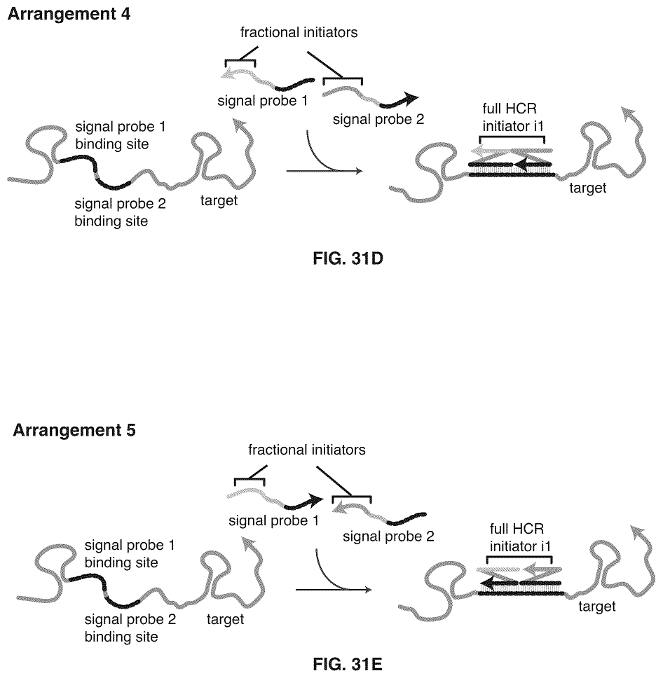

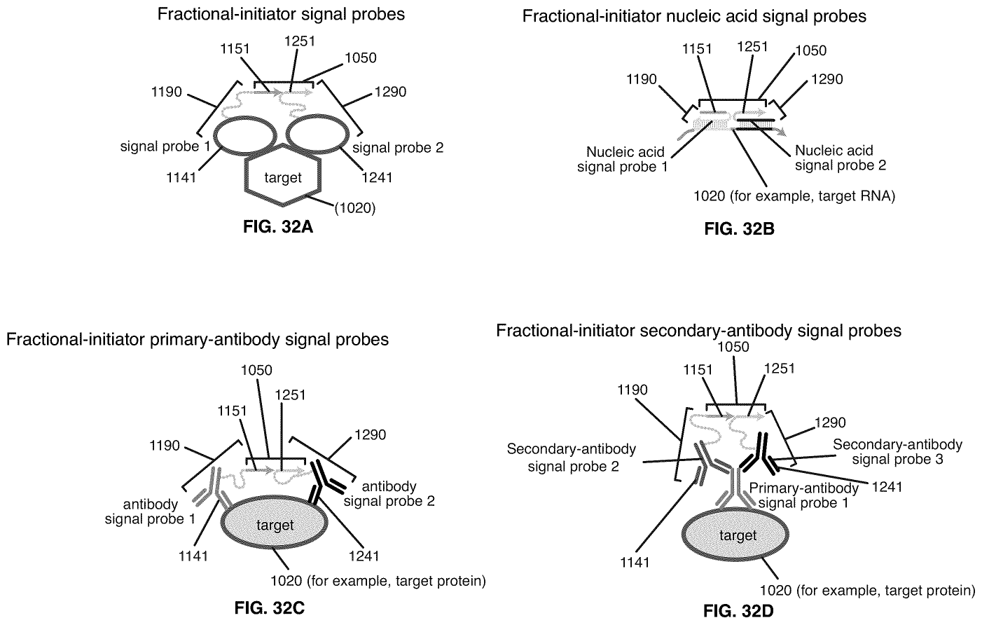

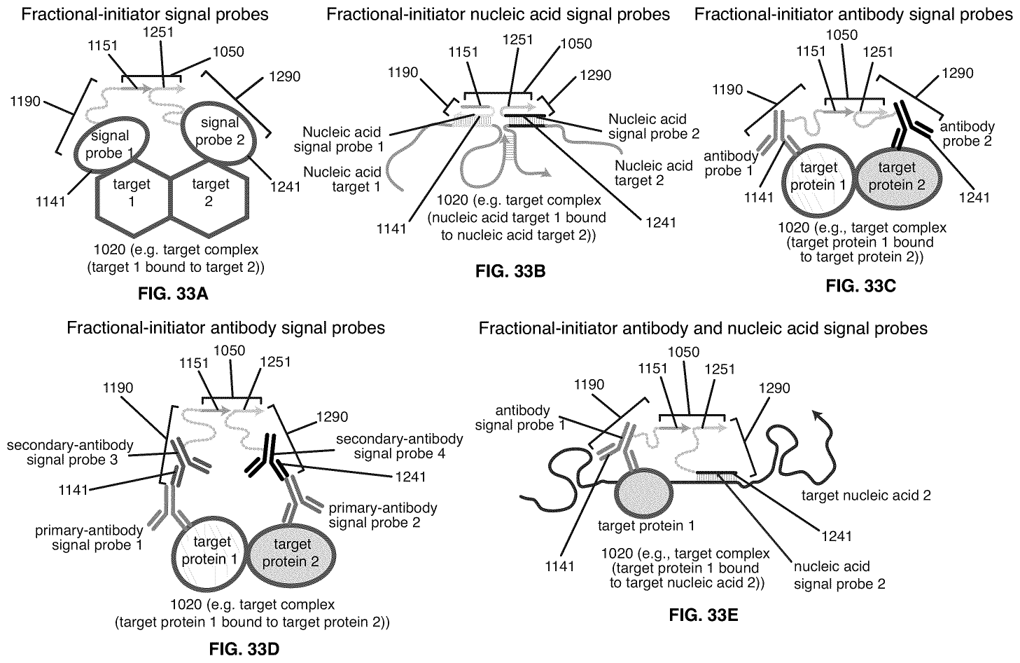

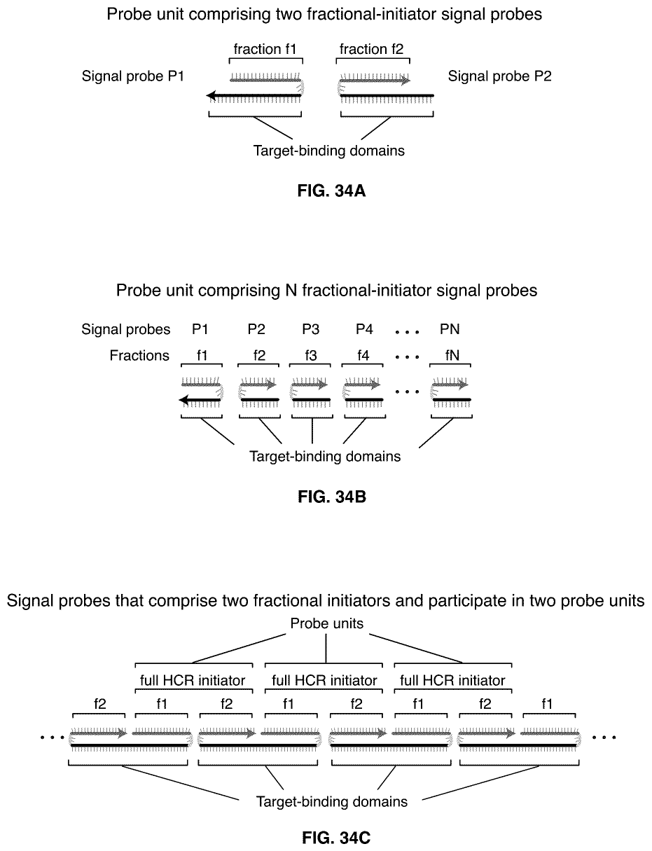

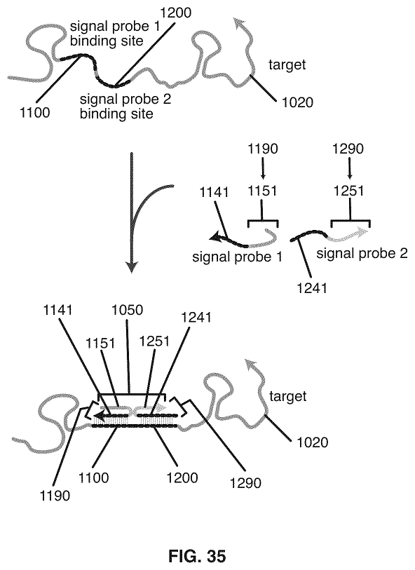

In some embodiments, a target is detected using a signal probe set comprising one or more initiator-labeled probes each comprising a target-binding domain and an amplification domain comprising one or more HCR initiators (for example, FIGS. 28A-28N and 29A-92F). In some embodiments, a target is detected within a sample using a signal probe set comprising one or more probe units (for example see the probe sets of FIGS. 5A-5B and 30A-30D), where a probe unit comprises two or more fractional-initiator probes (for example see the probe units of FIGS. 31A-31E, 32A-32D, 33A-33E, and 34A-34C), where each fractional-initiator probe comprises a target-binding domain and an amplification domain comprising a fractional initiator (for example, see the fractional-initiator probes of FIGS. 35 and 34A-34C). In some embodiments, binding of each probe within a probe unit to proximal cognate binding sites on the target colocalizes the fractional initiators to form a full HCR initiator (for example, see the full HCR initiators of FIGS. 5A-5B, 31A-31E, 32A-32D, 33A-33E, 30A-30D, and 36) capable of triggering HCR signal amplification. In some embodiments, each fractional-initiator probe within a probe unit further comprises a proximity domain (for example, see FIGS. 6, 7, 8, 9, 10, 11, 12, 13, 46A). In some embodiments, binding of each fractional-initiator probe within a probe unit to cognate binding sites on proximal targets enables binding of one or more proximity probes to the proximity domains within the probe unit to colocalize the fractional initiators to form a full HCR initiator (for example, see FIGS. 6, 7, 8, 9, 10, 11, 12, 13, 46A) capable of triggering HCR signal amplification.

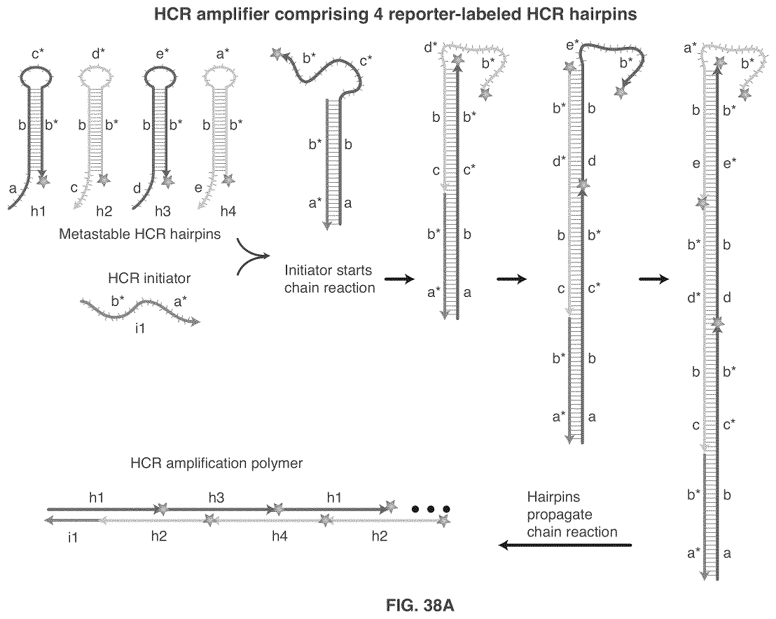

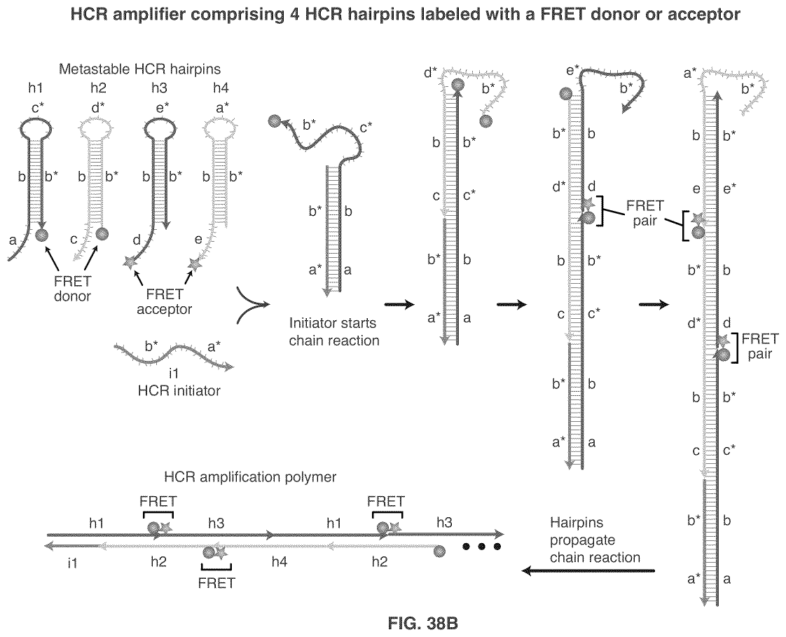

In some embodiments, the one or more HCR initiators on an initiator-labeled probe each initiate a chain reaction of polymerization steps in which the initiator hybridizes to the input domain of a first HCR hairpin, opening the first hairpin to expose its output domain, which in turn hybridizes to the input domain of a second HCR hairpin, opening the second hairpin to expose its output domain, and so on and so forth, leading to a chain reaction in which hairpins polymerize to yield an HCR amplification polymer tethered to the target (for example, see the amplification polymers of FIGS. 39, 40, 38A-38B, 41A, 41C-41E, and 42A). In some embodiments, in the absence of a full HCR initiator, HCR hairpins are kinetically trapped and do not polymerize, suppressing background. However, if the fractional-initiator probes within a probe unit bind to their proximal cognate binding sites on the target to colocalize a full HCR initiator (for example, see FIGS. 5A, 31A-31E, 32A-32D, 37C-37F), or if the proximity domains within the fractional-initiator probes within a probe unit bind to a proximity probe to colocalize a full HCR initiator (for example, see FIGS. 6, 7, 8, 9, 10, 11, 12, 13, 46A), the full HCR initiator initiates a chain reaction of polymerization steps in which the full initiator hybridizes to the input domain of a first HCR hairpin, opening the first hairpin to expose its output domain, which in turn hybridizes to the input domain of a second HCR hairpin, opening the second hairpin to expose its output domain, and so on and so forth, leading to a chain reaction in which hairpins polymerize to yield a tethered HCR amplification polymer (for example, see the amplification polymers of FIGS. 5A, 37C-37F, 38A-38B, 46A).

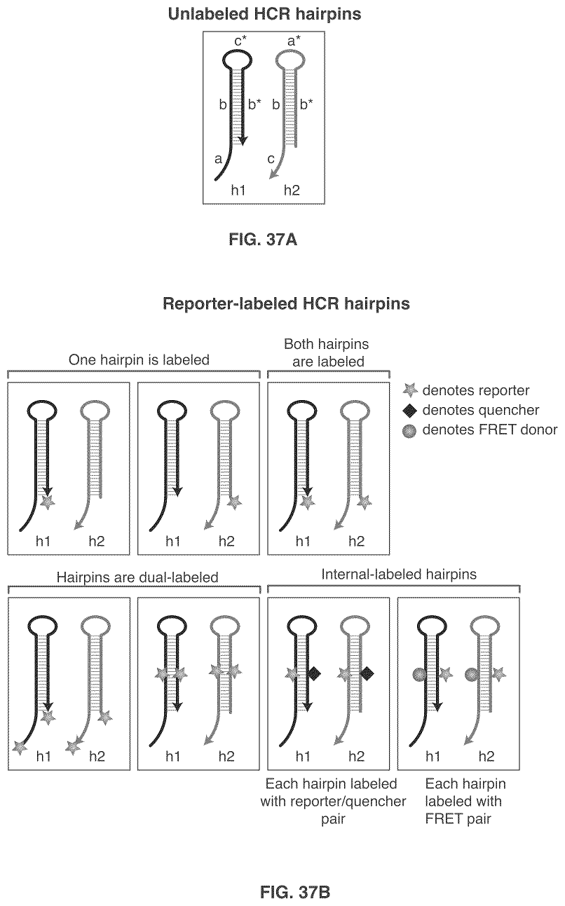

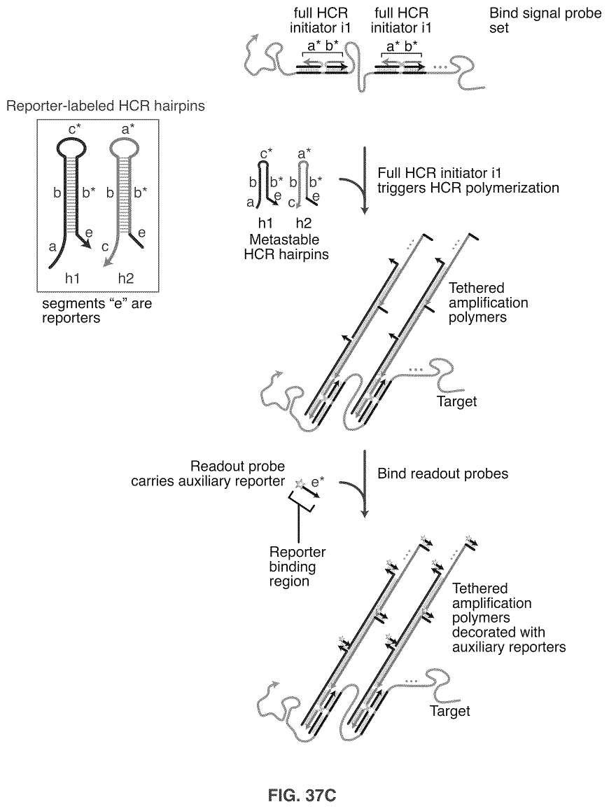

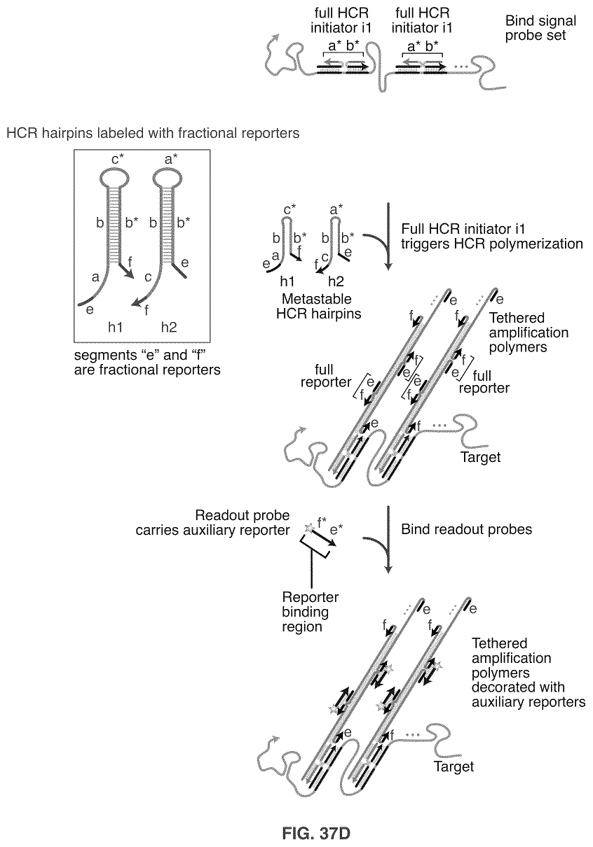

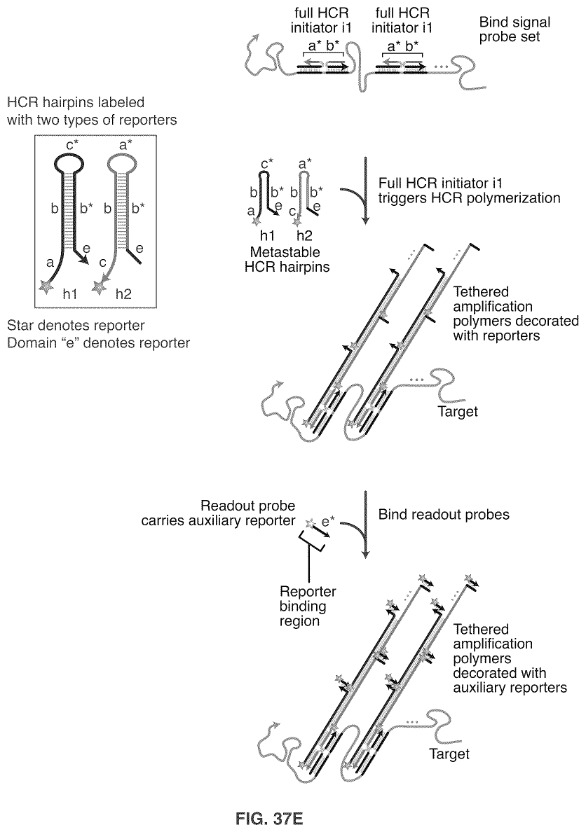

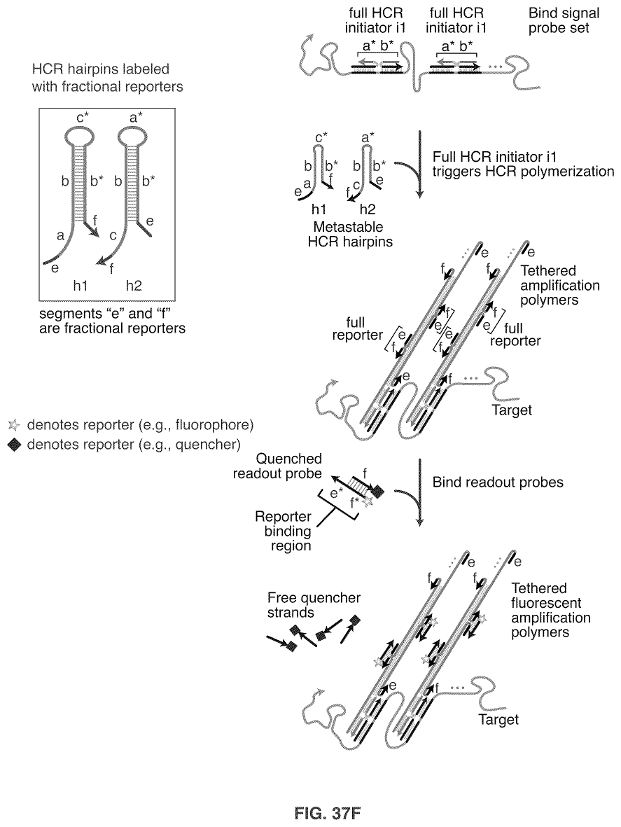

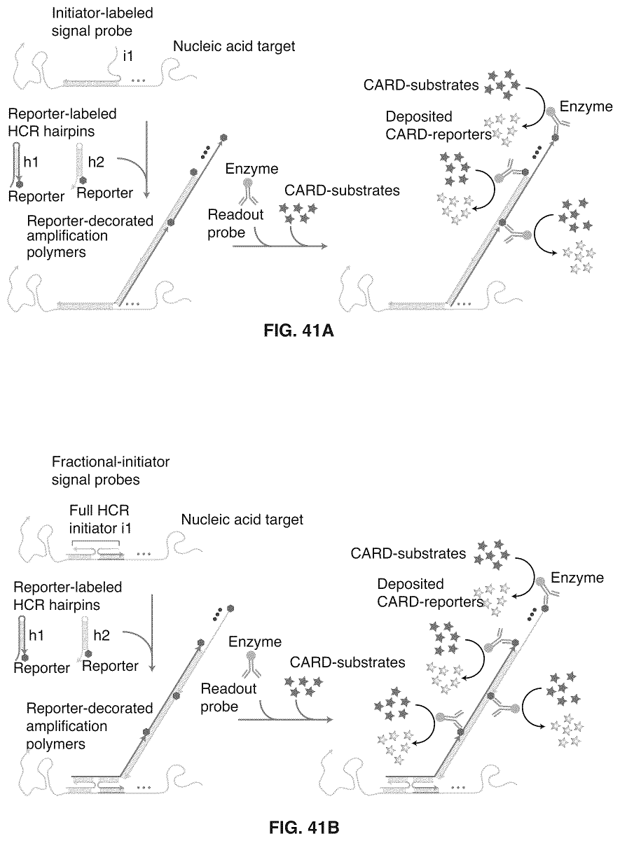

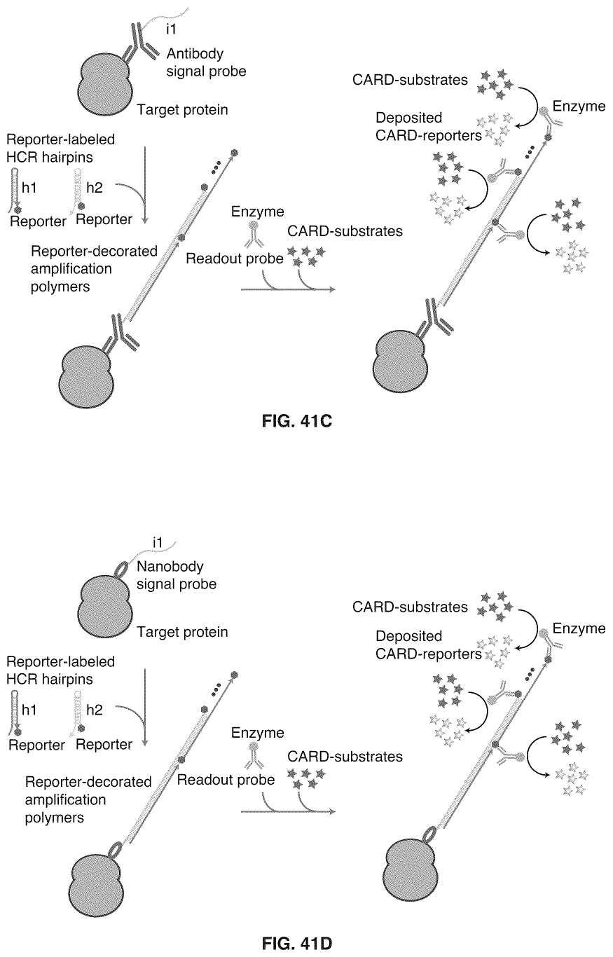

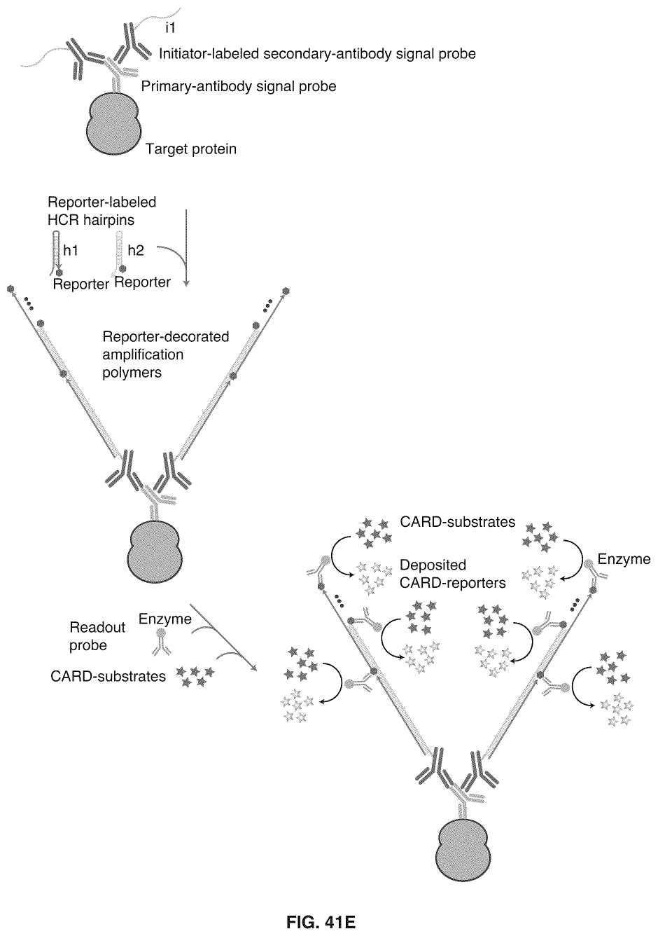

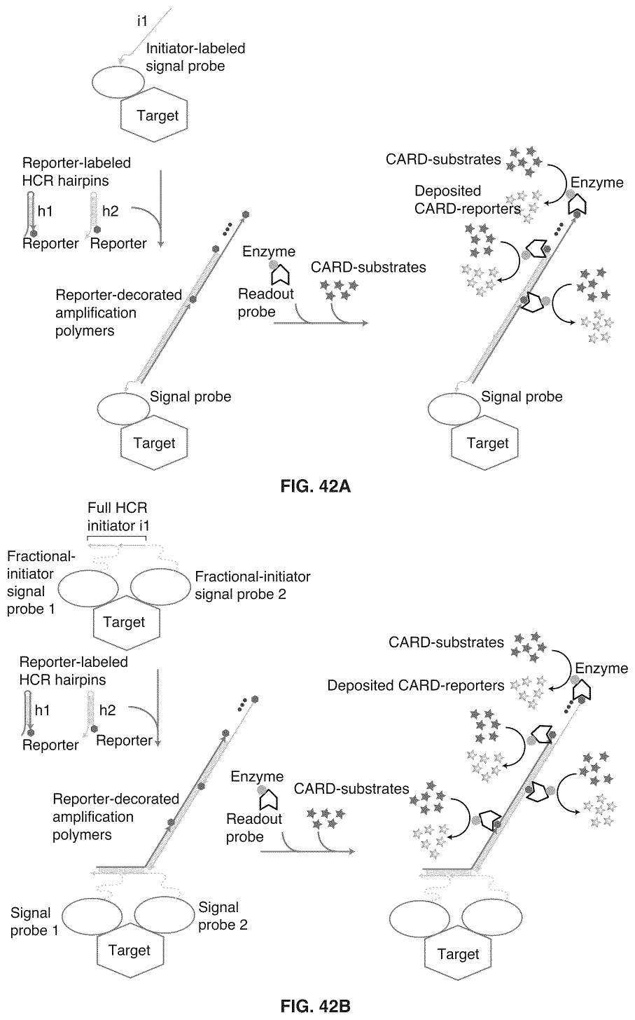

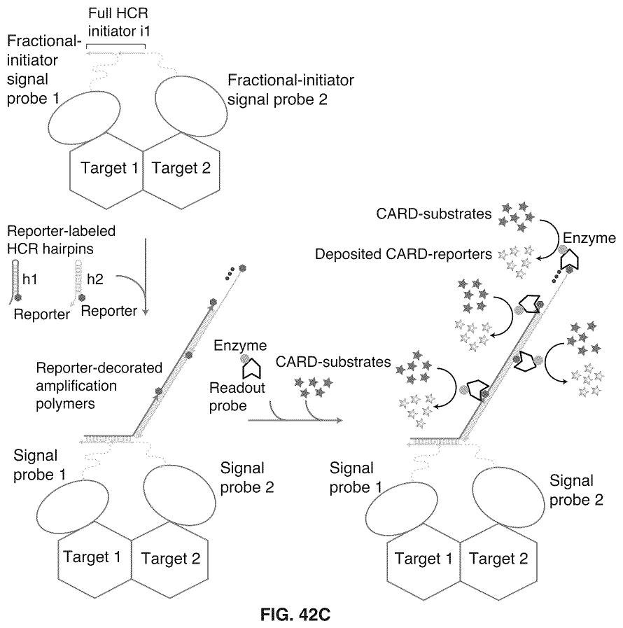

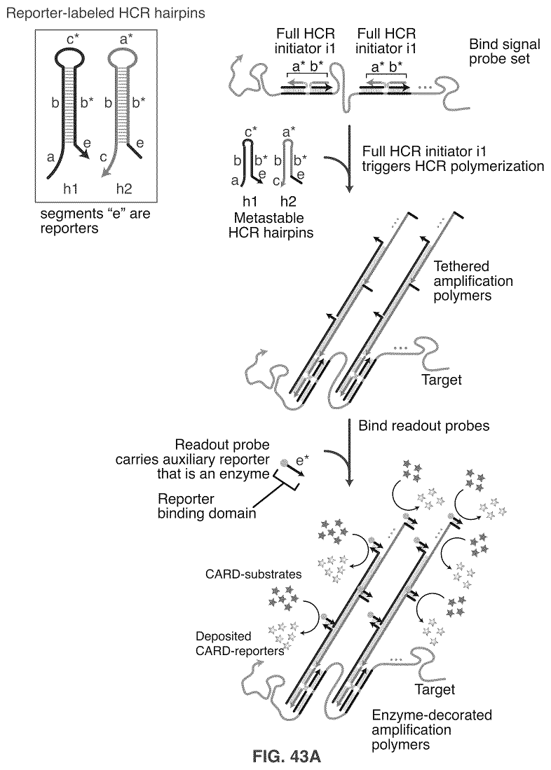

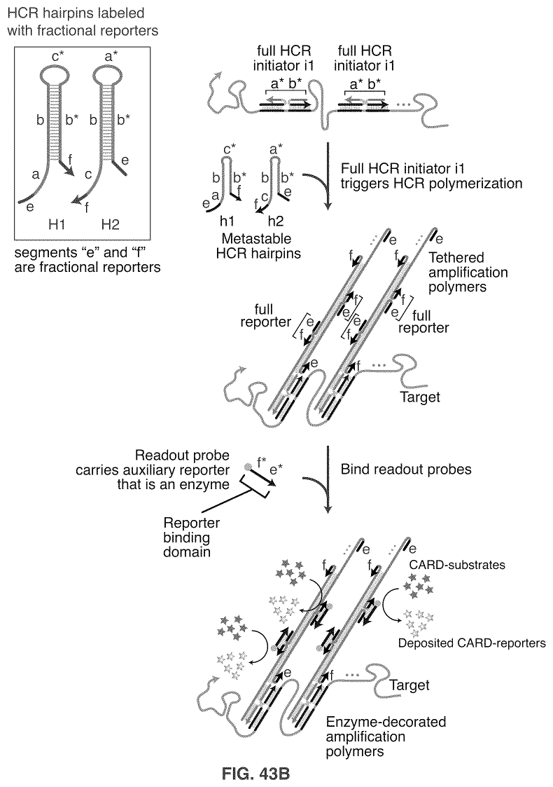

In some embodiments, an HCR hairpin further comprises zero, one, or more reporters that directly or indirectly lead to generation of an amplified signal (for example, see FIG. 37A-37F). In some embodiments, the zero, one, or more reporters on an HCR hairpin serve to mediate an additional layer of signal amplification via catalytic reporter deposition (CARD) (see for example, FIGS. 41A-41E, 42A-42C). In some embodiments, a reporter on an HCR hairpin comprises a fractional reporter such that an auxiliary-reporter-labeled readout probe does not strongly bind the fractional reporter on an individual hairpin, but such that following HCR polymerization, neighboring hairpins in the HCR amplification polymer colocalize a full reporter such that the colocalized full reporter strongly binds an auxiliary-reporter-labeled readout probe (for example, FIGS. 37D and 43B). In some embodiments, a readout probe comprises one or more auxiliary reporters and further comprises a reporter-binding domain configured to bind a reporter on an HCR amplification polymer or configured to bind a full reporter colocalized within an HCR amplification polymer (for example, see the readout probes of FIGS. 43A-43B). In some embodiments, amplified signal is generated by one or more reporters or auxiliary reporters associated with an HCR amplification polymer tethered to the target within the sample. In some embodiments, signal is removed. In some embodiments, HCR signal is generated, detected, and removed one or more times.

In some embodiments, HCR signal amplification increase the signal strength by a factor of 2, 5, 10, 15, 20, 25, 30, 40, 50, 75, 100, 500, 1000, 2000, 5000, 10,000, or 20,000-fold, or a value with a range defined by any two of the aforementioned values.

HCR initiators. In some embodiments, a colocalized full initiator comprises two or more fractional initiators brought into proximity by the proximity probe. In some embodiments, the full initiator is fully complementary to the input domain of an HCR hairpin such that it hybridizes to the input domain of the hairpin to open the hairpin and initiate the HCR polymerization cascade. In some embodiments, the full initiator is partially complementary to the input domain of an HCR hairpin, but sufficiently complementary such that it hybridizes to the input domain of the hairpin to open the hairpin and initiate the HCR polymerization cascade. In some embodiments, the full initiator is shorter or longer than the input domain of an HCR hairpin and/or has incomplete complementarity to the input domain of the hairpin, but is able to hybridize to the input domain of the hairpin to open the hairpin and initiate the HCR polymerization cascade. In some embodiments, a full initiator might have 60%, 70%, 80%, 90%, or 100% (or any intermediate value between any of these values) complementarity to the input domain of an HCR hairpin, and hybridize to the input domain of the hairpin to open the hairpin and initiate the HCR polymerization cascade. In some embodiments, a fractional initiator is shielded by base-pairing to reduce non-specific binding of probes within the sample. In some embodiments, a fractional initiator can be shielded by a hairpin structure. In some embodiments, a fractional initiator can be shielded by one or more auxiliary oligos. In some embodiments, a fractional initiator can be shielded by self-complementarity within a probe and/or complementarity to one or more auxiliary strands.

Automatic background suppression with HCR fractional-initiator probes. In some embodiments, fractional-initiator probes automatically suppress background because the HCR initiator is split between a pair of probes. In some embodiments, if fractional-initiator probes bind specifically to proximal targets and to a proximity probe, the proximity probe colocalizes the two fractional initiators within a probe pair to form a full HCR initiator. In some embodiments, individual fractional-initiator probes that bind non-specifically do not trigger HCR since each probe carries only a fraction of an HCR initiator, and HCR signal amplification is triggered only if the full HCR initiator is colocalized.

Automatic background suppression with HCR hairpins. In some embodiments, HCR hairpins automatically suppress background because HCR hairpins are kinetically trapped so they do not polymerize in the absence of an HCR initiator. In some embodiments, if both probes within a fractional-initiator probe pair bind specifically to their proximal cognate binding sites on the target (for example, see FIG. 5A) and/or on a proximity probe (for example, see FIG. 46A), the resulting colocalized full HCR initiator triggers growth of a tethered HCR amplification polymer. In some embodiments, individual HCR hairpins that bind non-specifically do not trigger HCR since they are kinetically trapped.

Automatic background suppression with HCR fractional-initiator probes and HCR hairpins. In some embodiments, the combination of HCR fractional-initiator probes for target detection and HCR amplification hairpins for signal amplification provides automatic background suppression throughout the protocol, ensuring that reagents will not generate amplified background even if they bind non-specifically.

Full HCR initiator formed by colocalization of 2 or more fractional-initiator probes. Each set of fractional-initiator probes that generate a full HCR initiator is referred to as a probe unit (for example, see FIG. 34). In some embodiments, a full HCR initiator is generated by a pair of fractional-initiator probes that each carry a fraction of the full HCR initiator such that together they comprise the full HCR initiator (fraction f1 for probe P1 and fraction f2 for probe P2 such that f1+f2=1); in this case, a probe unit is two fractional-initiator probes (for example, see FIG. 34A). In some embodiments, the fractions f1 and f2 are sufficiently small compared to the full HCR initiator (for example, f1=0.5 with f2=0.5; or f1=0.45 with f2=0.55; or f1=0.4 with f2=0.6; or f1=0.3 with f2=0.7) such that HCR signal amplification is suppressed if the full HCR initiator is not colocalized by the target.

In some embodiments, an HCR initiator (i1 or i2) is split between three fractional-initiator probes (fraction f1 for probe P1, fraction f2 for probe P2, fraction f3 for probe 3 such that f1+f2+f3=1); in this case, a probe unit consists of three fractional-initiator probes. In some embodiments, an HCR initiator (i1 or i2) is split between N fractional-initiator probes (fraction f1 for probe P1, fraction f2 for probe P2, . . . , fraction fN for probe PN such that f1+f2+ . . . fN=1; for example, see FIG. 15B) with N=2, 3, 4, or more; in this case, a probe unit consists of N fractional-initiator probes. In some embodiments, for any of these values of N, HCR signal amplification is suppressed if the full HCR initiator is not colocalized by the target.

In some embodiments, a full HCR initiator is generated by colocalization of a pair (or set) of probes that each carry a fraction of an HCR initiator such that the sum of the fraction f1 for probe P1 and the fraction f2 for probe P2 (f1+f2) is sufficiently close to 1 (for example, f1=0.47, f2=0.47, f1+f2=0.94; or f1=0.44, f2=0.42, f1+f2=0.86) such that HCR signal amplification is triggered by the colocalized full initiator that results from binding of the pair of probes to their cognate binding sites on proximal targets and binding of their proximity domains to a proximity probe. In some embodiments, the fractional-initiator probes within a probe unit generate a full HCR initiator corresponding to 100% of an HCR initiator. In some embodiments, the fractional-initiator probes within a probe unit generate a sufficient fraction of an HCR initiator to provide efficient HCR signal amplification relative to the rate of signal amplification when no fractional-initiator probes are present or when individual fractional-initiator probes are present but are not colocalized. In some embodiments, the fraction of a full HCR initiator generated by colocalized probes within a probe unit is 99%, 95%, 90%, 80%, or 60%, including any range above any one of the preceding values or defined between any two of the preceding values of a full HCR initiator. In some embodiments, a probe unit comprises 2, 3, 4, 5 or more fractional-initiator probes. In some embodiments, the fractional initiators in the probe unit are sufficient to be functional as an HCR initiator when the probes within the probe unit are colocalized by binding to their cognate binding sites on proximal targets and binding of their proximity domains to a proximity probe. In some embodiments, while an HCR initiator may have a sequence of a particular length (e.g., 15 nucleotides), the fractional initiators within a probe unit need not be the exact same length. For example, in some embodiments, their combined length could be 14 or 13 nucleotides, if, when colocalized, they still function as an HCR initiator.

In some embodiments, any two or more fractional initiators can be used, as long as, together, they provide the function of an HCR initiator.

In some embodiments, a full HCR initiator is generated by colocalization of a pair of probes that each carry a fraction of an HCR initiator further comprising one or a few or several sequence modifications such that the sum of the fraction f1 for probe P1 and the fraction f2 for probe P2 (f1+f2) is sufficiently close to 1 (for example, f1=0.45, f2=0.47, f1+f2=0.92) such that HCR signal amplification is triggered by the colocalized full initiator that results from binding of the pair of probes to their cognate binding sites on proximal targets and a proximity probe. In some embodiments, the fractional-initiator probes within a probe unit generate a full HCR initiator that has 100% sequence identity with an HCR initiator. In some embodiments, the fractional-initiator probes within a probe unit generate sufficient sequence identity to an HCR initiator to allow efficient HCR signal amplification relative to the rate of signal amplification when no fractional-initiator probes are present or when individual fractional-initiator probes are present but are not colocalized. In some embodiments, the full HCR initiator generated by colocalized probes within a probe unit has 99%, 95%, 90%, 80%, or 60% sequence identity with an HCR initiator, including any range above any one of the preceding values or defined between any two of the preceding values.

HCR amplifiers with 2 hairpins. In some embodiments, an HCR amplifier comprises two hairpin nucleic acids (h1 and h2; for example, see FIGS. 5A-5B and 37A-37F). In some embodiments, each hairpin comprises an input domain with a single-stranded toehold and a stem section, and an output domain with a single-stranded loop and a complement to the stem section. In the absence of an HCR initiator (i1 or i2), hairpins h1 and h2 coexist metastably, that is, they are kinetically trapped and do not polymerize.

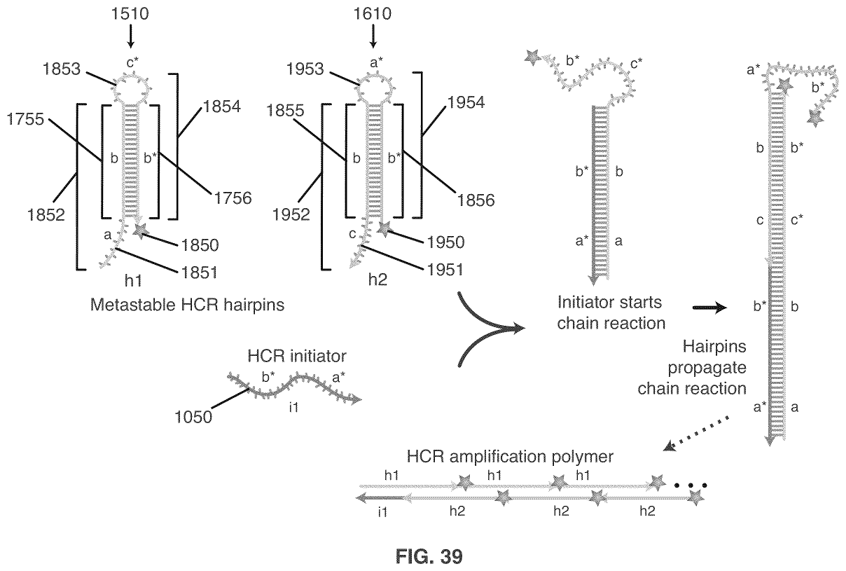

Initiation with initiator i1. In some embodiments, a full initiator i1 (1050) formed from colocalization of two or more fractional initiators comprises a domain complementary to the toehold of hairpin h1 (1851) and a domain complementary to the stem section of h1 (1755) (for example, see FIG. 39). In some embodiments, if an h1 hairpin (1510) encounters full initiator i1 (1050), the full initiator i1 hybridizes to the input domain of hairpin h1 (1852) via toehold-mediated strand displacement, opening hairpin h1 (1510) to expose the output domain of hairpin h1 (1854) and form complex i1-h1. In some embodiments, the output domain of hairpin h1 (1854) comprises a domain complementary to the toehold of hairpin h2 (1951) and a domain complementary to the stem section of h2 (1855). In some embodiments, if an h2 hairpin (1610) encounters an i1-h1 complex, the exposed output domain of h1 (1854) hybridizes to the input domain of hairpin h2 (1952) via toehold-mediated strand displacement, opening hairpin h2 to expose the output domain of hairpin h2 (1854) and form complex i1-h1-h2. In some embodiments, the output domain of hairpin h2 (1854) comprises a domain complementary to the toehold of hairpin h1 (1851) and a domain complementary to the stem section of h1 (1755). In some embodiments, if an h1 hairpin (1510) encounters an i1-h1-h2 complex, the exposed output domain of h2 (1854) hybridizes to the input domain of hairpin h1 (1852) via toehold-mediated strand displacement, opening hairpin h1 (1510) to expose the output domain of hairpin h1 (1854) and form complex i1-h1-h2-h1. In some embodiments, this polymerization process can repeat with alternating h1 and h2 polymerization steps to generate polymers of the form i1-h1-h2-h1-h2-h1-h2- . . . , which may be denoted i1-(h1-h2)_{N} for a polymer that incorporates N alternating copies of hairpins h1 and h2. For example, a polymer might incorporate several h1 and h2 molecules, or dozens of h1 and h2 molecules, or hundreds of h1 and h2 molecules, or thousands of h1 and h2 molecules, or tens of thousands of h1 and h2 molecules, or more. In some embodiments, it is possible for a polymer to end with either h1 or h2, so i1-(h1-h2)_{N}-h1 and i1-(h1-h2)_{N}-h1-h2 are both possible, the latter being equivalent to i1-(h1-h2)_{N+1}.

Initiation with initiator i2. In some embodiments, a full initiator i2 formed from colocalization of two or more fractional initiators comprises a domain complementary to the toehold of hairpin h2 and a domain complementary to the stem section of h2. In some embodiments, if an h2 hairpin encounters full initiator i2, the full initiator i2 hybridizes to the input domain of hairpin h2 via toehold-mediated strand displacement, opening hairpin h2 to expose the output domain of hairpin h2 and form complex i2-h2. In some embodiments, if an h1 hairpin encounters an i2-h2 complex, the exposed output domain of h2 hybridizes to the input domain of hairpin h1 via toehold-mediated strand displacement, opening hairpin h1 to expose the output domain of hairpin h1 and form complex i2-h2-h1. In some embodiments, if an h2 hairpin encounters an i2-h2-h1 complex, the exposed output domain of h1 hybridizes to the input domain of hairpin h2 via toehold-mediated strand displacement, opening hairpin h2 to expose the output domain of hairpin h2 and form complex i2-h2-h1-h2. In some embodiments, this polymerization process can repeat with alternating h2 and h1 polymerization steps to generate polymers of the form i2-h2-h1-h2-h1-h2-h1 . . . , which can be denoted i2-(h2-h1)_{N} for a polymer that incorporates N alternating copies of h2 and h1. For example, a polymer might incorporate several h1 and h2 molecules, or dozens of h1 and h2 molecules, or hundreds of h1 and h2 molecules, or thousands of h1 and h2 molecules, or tens of thousands of h1 and h2 molecules, or more. In some embodiments, it is possible for a polymer to end with either h1 or h2, so i2-(h2-h1)_{N}-h2 and i2-(h2-h1)_{N}-h2-h1 are both possible, the latter being equivalent to i2-(h2-h1)_{N+1}.

HCR amplifiers with 4 hairpins. In some embodiments, an HCR amplifier can comprise more than 2 hairpins. For example, an HCR amplifier might comprise 4 hairpins h1, h2, h3, h4 (for example, see FIGS. 38A and 38B). In some embodiments, just as for 2-hairpin HCR, each hairpin comprises an input domain comprising a single-stranded toehold and a stem section, and an output domain comprising a single-stranded loop and a complement to the stem section. In some embodiments, in the absence of a full HCR initiator (i1, i2, i3, or i4), hairpins h1, h2, h3, h4 coexist metastably, that is, they are kinetically trapped and do not polymerize. In some embodiments, the output domain of hairpin h1 comprises a domain complementary to the toehold of hairpin h2 and a domain complementary to the stem section of h2; the output domain of hairpin h2 comprises a domain complementary to the toehold of hairpin h3 and a domain complementary to the stem section of h3; the output domain of hairpin h3 comprises a domain complementary to the toehold of hairpin h4 and a domain complementary to the stem section of h4; the output domain of hairpin h4 comprises a domain complementary to the toehold of hairpin h1 and a domain complementary to the stem section of h1. In some embodiments, full initiator i1 formed from colocalization of two or more fractional initiators comprises a domain complementary to the toehold of hairpin h1 and a domain complementary to the stem section of h1; full initiator i2 formed from colocalization of two or more fractional initiators comprises a domain complementary to the toehold of hairpin h2 and a domain complementary to the stem section of h2; full initiator i3 formed from colocalization of two or more fractional initiators comprises a domain complementary to the toehold of hairpin h3 and a domain complementary to the stem section of h3; full initiator i4 formed from colocalization of two or more fractional initiators comprises a domain complementary to the toehold of hairpin h4 and a domain complementary to the stem section of h4. In some embodiments, analogous to the case of 2-hairpin HCR, if a hairpin h1 encounters a full initiator i1, the full initiator i1 opens hairpin h1 to form complex i1-h1 with an exposed h1 output domain, which in turn opens hairpin h2 to form complex i1-h1-h2 with an exposed h2 output domain, which in turn opens hairpin h3 with an exposed output domain to form complex i1-h1-h2-h3 with an exposed h3 output domain, which in turn opens hairpin h4 to form complex i1-h1-h2-h3-h4 with an exposed h4 output domain, which in turn opens hairpin h1 to form complex i1-h1-h2-h3-h4-h1 with an exposed h1 output domain, and so on and so forth, leading to polymerization via alternating h1, h2, h3, and h4 polymerization steps to generate polymers of the form i1-h1-h2-h3-h4-h1-h2-h3-h4-h1-h2-h3-h4 . . . , which can be denoted i1-(h1-h2-h3-h4)_{N} for a polymer that incorporates N alternating copies of h1, h2, h3, and h4. In some embodiments, it is possible for a polymer to end with h1, h2, h3, or h4, so i1-(h1-h2-h3-h4)_{N}-h1, i1-(h1-h2-h3-h4)_{N}-h1-h2, i1-(h1-h2-h3-h4)_{N}-h1-h2-h3, and i1- (h1-h2-h3-h4)_{N}-h1-h2-h3-h4 are all possible, the latter being equivalent to i1-(h1-h2-h3-h4)_{N+1}. In some embodiments, it is possible for HCR polymerization to be triggered by any of the cognate full initiators (i1, i2, i3, or i4). For example, initiation by full initiator i3 could generate polymers of the form i3-(h3-h4-h1-h2)_{N}. In some embodiments, HCR amplifiers with 4 hairpins are convenient for generating a signal that is absent in the unpolymerized state and present in the polymer state (for example, FIG. 19B illustrates FRET pairs that are colocalized to generate a FRET signal only when hairpins are colocalized within an amplification polymer, providing a basis for wash-free methods since unused hairpins that are not washed from the sample will not participate in FRET, and hence will avoid generating background).

HCR amplifiers with 2 or more hairpins. More generally, in some embodiments, an HCR amplifier may comprise M HCR hairpins (h1, h2, . . . , hM) with M an integer of 2 or more. In the absence of a full HCR initiator (i1, i2, . . . , iM) formed from colocalization of two or more fractional initiators, hairpins h1, h2, . . . , hM coexist metastably, that is, they are kinetically trapped and do not polymerize. In the presence of a cognate full HCR initiator formed from colocalization of two or more fractional initiators, polymerization occurs via alternating polymerization steps analogous to 2-hairpin or 4-hairpin HCR. For example, full initiator i1 would lead to growth of polymers of the form i1-(h1-h2- . . . -hM)_{N} for a polymer that incorporates N alternating copies of h1, h2, . . . , hM. It is possible for a polymer to end with any of h1, h2, . . . , hM, so i1-(h1-h2- . . . -hM)_{N}-h1, i1-(h1-h2- . . . -hM)_{N}-h1-h2, . . . , and i1-(h1-h2- . . . -hM)_{N}-h1-h2- . . . -hM are all possible, the latter being equivalent to i1-(h1-h2- . . . -hM)_{N}+1. It is possible for HCR polymerization to be triggered by any of the cognate full initiators (i1, i2, . . . , iM). For example, initiation by full initiator i3 could generate polymers of the form i3-(h3- . . . -hM-h1-h2)_{N}.

Reporter-labeled HCR hairpins. For a given HCR amplifier, each HCR hairpin comprises zero, one, or more reporters. Reporters on different hairpins within an amplifier may be the same or different. For example, an amplifier comprising hairpins h1 and h2 might have: 1) the same reporter on h1 and h2, 2) different reporters on h1 and h2, 3) a reporter on h1 but no reporter on h2, 4) a reporter on h2 but no reporter on h1, 5) no reporter on h1 or h2, 6) zero, one, or more reporters on h1 of which zero, one, or more of them are the same or different as zero, one, or more reporters on h2. Similarly, for an HCR amplifier comprising hairpins h1, h2, h3, h4, each hairpin may comprise zero, one, or more reporters (for example 3, 5, or 10 reporters) of which zero, one, or more of them may be the same as zero, one, or more reporters on each of the other hairpins. In some embodiments, one or more of the reporters for a given hairpin can be unique within a mixture of hairpins and/or hairpin reporters. In some embodiments, there are 1, 10, 100, 1000, 10,000, 100,000 or more unique reporters within a mixture (including any range defined between any two of the previous numbers).

In some embodiments, the one or more reporters on a reporter-labeled HCR hairpin directly or indirectly contributes to the generation, alteration, or elimination of a signal. For example, a reporter could be a fluorophore, a chromophore, a luminophore, a phosphor, a FRET pair, a member of a FRET pair, a quencher, a fluorophore/quencher pair, a rare-earth element or compound, a radioactive molecule, a magnetic molecule, an enzyme, or any other molecule that directly or indirectly facilitates measurement of a signal.

In some embodiments, a reporter decorating a tethered HCR amplification polymer may bind to the reporter-binding domain of a readout probe to directly or indirectly mediate localization of auxiliary reporters in the vicinity of the reporter, which in turn directly or indirectly mediates generation of an amplified signal. For example:

In some embodiments, the reporter can comprise digoxigenin (DIG) that recruits anti-DIG antibody as the readout probe, where the anti-DIG is directly labeled with one or more auxiliary reporters, or with one or more reporters that serve to directly or indirectly mediate localization of auxiliary reporters in the vicinity of the reporter.

In some embodiments, the reporter can comprise a nucleic acid domain that serves as a substrate with full or partial sequence complementarity to a reporter-binding domain within a readout probe that carries one or more auxiliary reporters (for example, see FIG. 37C),

In some embodiments, the reporter can comprise a nucleic acid domain that serves as a substrate with full or partial sequence complementarity to a reporter-binding domain within a readout probe that carries one or more substrates that serve to mediate localization of auxiliary reporters in the vicinity of the reporter.

In some embodiments, the reporter can comprise a nucleic acid domain that serves as a substrate for a readout probe that directly or indirectly mediates localization of auxiliary reporters in the vicinity of the reporter.

In some embodiments, the reporter can comprise a substrate that serves to recruit a readout probe that indirectly mediates localization of auxiliary reporters in the vicinity of the reporter.

In some embodiments, the reporter can comprise a substrate that serves to recruit a readout probe that comprises an enzyme that mediates catalytic reporter deposition (CARD) in the vicinity of the reporter (for example, see FIG. 43A).

In some embodiments, the reporter can comprise biotin that recruits streptavidin (or another biotin-binding molecule) as the readout probe, where the streptavidin is directly labeled with one or more auxiliary reporters, or with one or more substrates that serve to directly or indirectly mediate localization of auxiliary reporters in the vicinity of the reporter.

In some embodiments, the reporter can comprise a hapten that recruits an anti-hapten antibody readout probe or an anti-hapten nanobody readout probe that directly or indirectly mediates localization of reporters in the vicinity of the reporter via CARD signal amplification. For example, the anti-hapten antibody or nanobody readout probe may comprise an enzyme that mediates CARD (for example, see FIGS. 41A-41E).

In some embodiments, the reporter can comprise a hapten that recruits an anti-hapten that directly or indirectly mediates localization of auxiliary reporters in the vicinity of the reporter. For example, the anti-hapten readout probe may comprise an enzyme that mediates CARD (for example, see FIGS. 42A-42C).

In some embodiments, the reporter can comprise an enzyme that mediates CARD signal amplification to deposit CARD-reporter molecules in the vicinity of the hairpin.

In some embodiments, the reporter can comprise zero, one, or more haptens (for example, see FIGS. 37A-37B) that mediate, directly or indirectly, localization of auxiliary reporters in the vicinity of the haptens.

In some embodiments, the reporter can comprise a hapten that recruits an anti-hapten (for example, an antibody, a nanobody, streptavidin, or another molecule) that is labeled with auxiliary reporters.

In some embodiments provided herein, HCR signal amplification is used to mediate catalytic reporter deposition (CARD), leading to even higher signal gain. In some embodiments, the even higher single gain is about 5, 10, 15, 20, 25, 30, 40, 50, 75, 100, 500, 1000, 2000, 5000, 10,000, 20,000, 50,000, or 100,000-fold, or a value with a range defined by any two of the aforementioned values.

Haptens and anti-haptens. In some embodiments, hairpin labels that are substrates comprising a hapten could for example be digoxygenin (DIG), dinitrophenyl (DNP), a fluorophore, biotin, or any small molecule, biological molecule, or non-biological molecule that can recruit an anti-hapten. Examples of anti-haptens include antibodies, nanobodies, streptavidin, aptamers, or any other molecule or complex of molecules that selectively binds a hapten.

Enzymes for HCR-mediated Catalytic Reporter Deposition (CARD). In some embodiments, reporter-decorated HCR amplification polymers mediate signal amplification via catalytic reporter deposition (CARD) by an enzyme that catalyzes a CARD-substrate leading to deposition of CARD-reporters in the vicinity of the HCR amplification polymer (for example, see FIGS. 41A-41E, 42A-42C, 43A-43B). For example:

In some embodiments, the enzyme could be horseradish peroxidase (HRP) (or polymer HRP comprising multiple HRP enzymes) that acts on a CARD-substrate to catalyze deposition a chromogenic CARD-reporter such as AEC, DAB, TMB, or StayYellow, or that catalyzes a CARD-substrate to catalyze deposition of a fluorescent CARD-reporter such as fluorophore-labeled tyramide, or that catalyzes deposition of a hapten-labeled CARD-substrate such as biotin-labeled tyramide, where the hapten serves to mediate localization of CARD-reporters in the vicinity of the reporter-decorated HCR amplification polymer.

In some embodiments, the enzyme could be alkaline phosphatase (AP) (or polymer AP comprising multiple AP enzymes) that acts on a CARD-substrate to catalyze deposition of CARD-reporters, for example a chromogenic CARD-reporter such as but not limited to BCIP/NBT, BCIP/TNBT, Napthol AS-MX phosphate+FastBlue BB, Napthol AS-MX phosphate+FastRed TR, StayGreen.

In some embodiments, the enzyme could be glucose oxidase that acts on a CARD-substrate to catalyze deposition of CARD-reporters, for example NBT.

In some embodiments, the enzyme could be any molecule or complex that directly or indirectly mediates localization of CARD-reporters in the vicinity of a reporter-decorated HCR amplification polymer.

In some embodiments, the enzyme that mediates CARD is deactivated (aka inactivated) after CARD-reporter deposition (for example, using chemical or heat denaturation). For example, the enzyme that mediates CARD can be deactivated using any combination of:
1. Heat (for example, 65° C. or above)
2. Fixative (for example, 4% PFA)
3. Acid (for example, 0.1 M glycine-HCl with 1% Tween 20 at pH 2.2, 0.2N HCl, 10% acetic acid, 10 mM HCl)
4. Other chemicals (for example, hydrogen peroxide (H₂O₂), hydrogen peroxide+phenol, sodium azide, DEPC, MAB with 10 mM EDTA)

In some embodiments, HRP is inactivated using H₂O₂. In some embodiments, AP is inactivated using a combination of heat and acid. In some embodiments, AP is inactivated with fixative. In some embodiments, deactivation of the enzyme that mediates CARD allows repeated CARD using the same enzyme in combination with different substrates for different targets to allow multiplexed target analysis using HCR-mediated CARD. In some embodiments, deactivation of the enzyme that mediates CARD allows repeated CARD using different enzymes in combination with different CARD-substrates for different targets to allow multiplexed target analysis using HCR-mediated CARD.

In some embodiments, CARD allows storage of stained samples for 10 or more years to allow reanalysis in compliance with regulatory requirements. In some embodiments, the CARD-stained sample is adequately stable for 1, 2, 3, 4, 5, 6, 7, 8, 9, 10, 11, 12, 13, 14, 15 or more years, and still in compliance with regulatory requirements. In some embodiments, CARD staining provides for long-term storage of archival samples.

Target types. In some embodiments, a fractional-initiator probe comprises a target-binding domain, a fractional initiator, and a proximity domain. In some embodiments, a probe unit comprises two or more fractional-initiator probes such that when their target-binding domains are bound to proximal cognate targets and their proximity domains are bound to a cognate proximity probe (or probes), their fractional initiators are colocalized to form a full HCR initiator capable of triggering HCR.

In some embodiments, a probe unit can detect a target comprising any molecule including but not limited to an RNA molecule (for example, mRNA, rRNA, lncRNA, siRNA, shRNA, microRNA, non-coding RNA, synthetic RNA, or modified RNA), a DNA molecule, a non-natural nucleic acid molecule, a protein molecule, a small molecule, a biological molecule, a chemically modified biological molecule, a non-biological molecule;

In some embodiments, a probe unit can detect a target comprising any complex of molecules comprising any combination of RNA, DNA, protein, small molecules, biological molecules, and/or non-biological molecules (for example, an RNA:RNA complex, an RNA:protein complex, a DNA:protein complex, an RNA:DNA:protein complex, a protein:protein complex), a complex of 2, 3 or more molecules.

In some embodiments, a probe unit can detect a target comprising any collection of proximal molecules or complexes such that the fractional initiators in the probe unit can colocalize to form a full HCR initiator when the fractional-initiator probes comprising the probe unit are bound to their respective targets within the collection of proximal molecules or complexes and also to their cognate proximity probe (or probes).

In some embodiments, the target-binding domains within a probe unit are configured to bind to overlapping or non-overlapping regions of the target. In some embodiments, the proximity domains within a probe unit are configured to bind to overlapping or non-overlapping regions of the proximity probe. In some embodiments, the fractional initiators within a probe unit are designed to hybridize to overlapping or non-overlapping regions of an HCR hairpin. In some embodiments, individual fractional-initiator probes that bind non-specifically do not colocalize a full HCR initiator, suppressing spurious generation of amplified HCR background. In some embodiments, all of the fractional-initiator probes within a probe unit must bind to one or more proximity probes in order trigger HCR signal amplification.

In any of the embodiments provided herein, the fractional initiators within a probe unit are designed to be (or are) complementary to non-overlapping regions of an HCR hairpin (for example, regions separated by 0, 1, 2, or more nucleotides), or are designed to be (or are) complementary to overlapping regions of an HCR hairpin (for example, regions that overlap by 1, 2 or more nucleotides), or are designed to be (or are) substantially complementary to an HCR hairpin (for example, complementary except for 0, 1, 2, a few, or several mismatches), or are configured to bind an HCR hairpin.

In any of the embodiments provided herein, the target-binding regions within a probe unit are configured to bind to non-overlapping regions of the target (for example, regions separated by 0, 1, 2, or more nucleotides or regions separated by 0, 1, 2, or more nanometers), or are configured to bind to overlapping regions of the target (for example, regions that overlap by 1, 2 or more nucleotides, or regions that overlap by 1, 2, or more nanometers).

In any of the embodiments provided herein, the proximity domains within a probe unit are configured to bind to non-overlapping regions of one or more proximity probes (for example, regions separated by 0, 1, 2, or more nucleotides or regions separated by 0, 1, 2, or more nanometers), or are configured to bind to overlapping regions of one or more proximity probes (for example, regions that overlap by 1, 2 or more nucleotides, or regions that overlap by 1, 2, or more nanometers).

In some embodiments, a fractional-initiator probe comprises a kissing domain. In some embodiments, a probe unit comprises two fractional-initiator probes, each comprising a kissing domain. In some embodiments, the kissing domains within a probe unit are configured to bind to each other. In some embodiments, a probe unit comprises two or more fractional-initiator probes, each comprising a kissing domain. In some embodiments, the kissing domains within a probe unit are configured to bind to each other.

Fractional-initiator probes for multiplexing. In some embodiments, fractional-initiator probes are designed for multiplexed experiments in which 2, 3, 4, 5, 10, 20, or 100 or more fractional-initiator probes are used to bind to different targets in the same sample. In some embodiments, multiplexed experiments with fractional-initiator probes can be used to detect multiple target complexes, or multiple targets in proximity to one another within a sample.

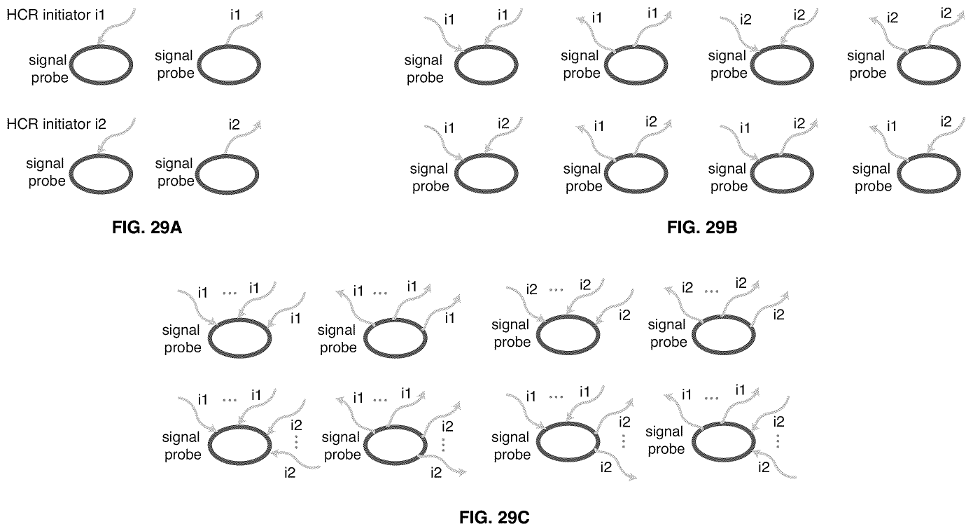

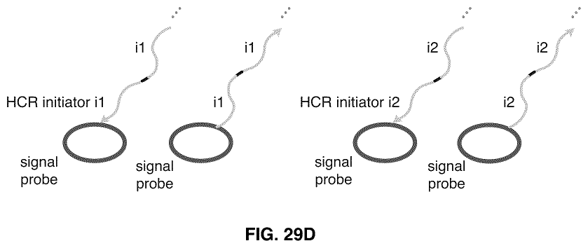

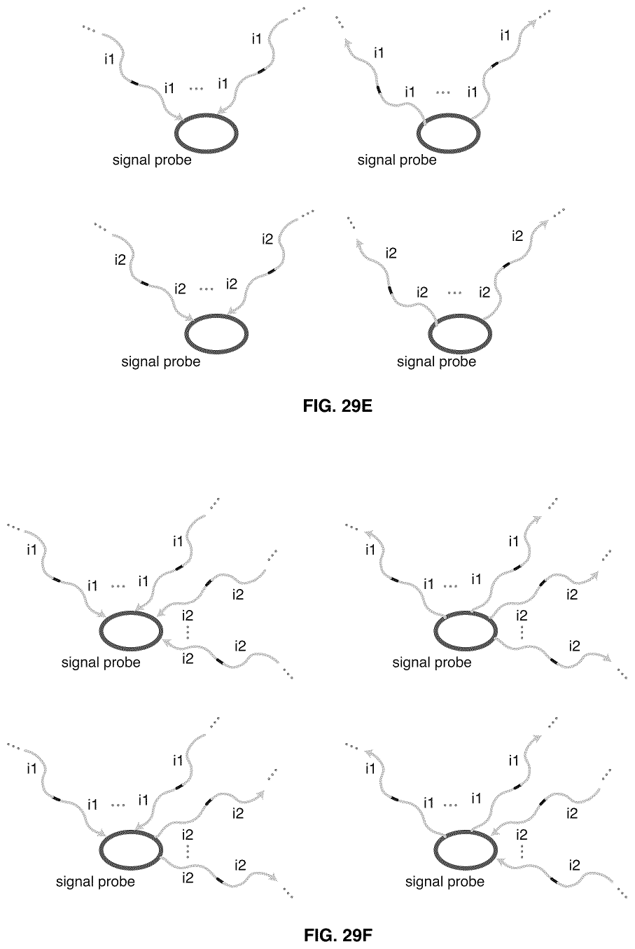

Materials and compositions of initiator-labeled probes. In some embodiments, an initiator-labeled probe comprises one or more target-binding domains and one or more HCR initiators (for example, see FIGS. 28A-28N and 29A-29F). In some embodiments, each domain may comprise one or more materials including DNA, RNA, 2′OMe-RNA, PNA, XNA, chemically modified nucleic acids, synthetic nucleic acid analogs, amino acids, chemical linkers, synthetic amino acid analogs, and/or any other molecule suited for the purpose of the domain. For example:

In some embodiments, an initiator-labeled probe may comprise one or more initiators made of DNA and a target-binding domain made of DNA.

In some embodiments, an initiator-labeled probe may comprise one or more initiators made of DNA, a chemical linker, and a target-binding domain made of amino acids (for example, an antibody or a nanobody or an antibody fragment).

In some embodiments, an initiator-labeled probe may comprise an initiator made of a synthetic nucleic acid analog and a target-binding domain made of a combination of DNA and 2′OMe-RNA.

In some embodiments, an initiator-labeled probe may comprise an initiator made of 2′OMe-RNA and a target-binding domain made of a combination of RNA and protein.

In some embodiments, an initiator-labeled probe may comprise an initiator made of DNA and a target-binding domain made of PNA.

In some embodiments, an initiator-labeled probe may comprise one or more initiators made of any nucleic acid or nucleic acid analog and one or more target-binding domains made of any combination of materials suitable for binding the target molecule.

In some embodiments, an initiator-labeled probe may comprise an antibody or nanobody conjugated to one or more oligonucleotides each comprising one or more initiators.

In some embodiments, an initiator-labeled probe may comprise a single covalently linked molecule or may comprise two or more molecules (each covalently linked) that interact non-covalently to form a complex. For example:

In some embodiments, an initiator-labeled probe may comprise an initiator made of DNA that is covalently linked to a target-binding domain made of DNA.

In some embodiments, an initiator-labeled probe may comprise an initiator made of a nucleic acid or nucleic acid analog that is covalently linked or non-covalently bound to a target-binding domain comprising one or more molecules.

In some embodiments, an initiator-labeled probe may comprise an antibody or nanobody conjugated to one or more oligonucleotides each comprising one or more fractional initiators, a proximity binding domain, wherein the antibody or nanobody is a secondary probe that binds to a primary probe that binds the target.

Materials and composition of fractional-initiator probes. In some embodiments, a fractional-initiator probe comprises one or more target-binding domains, one or more fractional initiators, and optionally one or more proximity domains (for example, see FIGS. 6, 7, 8, 9, 10, 11, 12, 13, 14, 31A-31E, 32A-32D, 33A-33E, 36, 46A). In some embodiments, each domain may comprise one or more materials including DNA, RNA, 2′OMe-RNA, PNA, XNA, chemically modified nucleic acids, synthetic nucleic acid analogs, chemical linkers, amino acids, synthetic amino acid analogs, and/or any other molecule suited for the purpose of the domain. For example:

In some embodiments, a fractional-initiator probe may comprise one or more fractional initiators made of DNA, an optional proximity domain made of DNA, and a target-binding domain made of DNA.

In some embodiments, a fractional-initiator probe may comprise one or more fractional initiators made of DNA, an optional proximity domain made of DNA, a chemical linker, and a target-binding domain made of amino acids (for example, an antibody or a nanobody or an antibody fragment).

In some embodiments, a fractional-initiator probe may comprise a fractional initiator made of a synthetic nucleic acid analog, an optional proximity domain made of synthetic nucleic acid analog, and a target-binding domain made of a combination of DNA and 2′OMe-RNA.

In some embodiments, a fractional-initiator probe may comprise a fractional initiator made of 2′OMe-RNA, and optional proximity domain made of 2′OMe-RNA, and a target-binding domain made of a combination of RNA and protein.

In some embodiments, a fractional-initiator probe may comprise a fractional initiator made of DNA, an optional proximity domain made of DNA, and a target-binding domain made of PNA.

In some embodiments, a fractional-initiator probe may comprise one or more fractional initiators made of any nucleic acid or nucleic acid analog, one or more optional proximity domains made of nucleic acid or nucleic acid analog, and one or more target-binding domains made of any combination of materials suitable for binding the target molecule.

In some embodiments, a fractional-initiator probe may comprise an antibody or a nanobody conjugated to an oligonucleotide comprising a fractional initiator and a proximity domain.

In some embodiments, a fractional-initiator probe may comprise a target-binding domain, a fractional initiator, and a proximity domain.

In some embodiments, a fractional-initiator probe may comprise a single covalently linked molecule or may comprise two or more molecules (each covalently linked) that interact non-covalently to form a complex. For example:

In some embodiments, a fractional-initiator probe may comprise a fractional initiator and an optional proximity domain made of DNA that is covalently linked to a target-binding domain made of DNA.

In some embodiments, a fractional-initiator probe may comprise one or more fractional initiators and one or more optional proximity domains made of DNA that are covalently linked to dCas9 (or another Cas) which is non-covalently bound to a guide RNA (gRNA) such that the target-binding domain comprises the gRNA:dCas9 complex (or gRNA:Cas complex using another Cas).

In some embodiments, a fractional-initiator probe may comprise one or more fractional initiators and one or more optional proximity domains made of DNA that are covalently linked to a gRNA that is non-covalently bound to dCas9 (or another Cas) such that the target-binding domain comprises the gRNA:dCas9 complex (or gRNA:Cas complex using another Cas).

In some embodiments, a fractional-initiator probe may comprise a fractional initiator and optional proximity domain made of a nucleic acid or nucleic acid analog that is covalently linked or non-covalently bound to a target-binding domain comprising one or more molecules. Each fractional-initiator probe within a probe unit may have the same or different material compositions from the other fractional-initiator probes in the probe unit. Each fractional-initiator probe within a probe unit may have target-binding regions that bind to different detection sites on the same target molecule, or to different detection sites within a target complex, or to different detection sites within a target collection of proximal molecules or complexes.

In some embodiments, a fractional-initiator probe may comprise an antibody or nanobody conjugated to one or more oligonucleotides each comprising a fractional initiator and optionally a proximity domain, wherein the antibody or nanobody is a secondary probe that binds to a primary probe that binds the target.

In some embodiments, a fractional-initiator probe may comprise a target-binding domain, a proximity-domain, and a fractional initiator, wherein the target is a primary probe that binds a primary target.

Removal of signal from the sample. In some embodiments, HCR signal is removed from the target after detecting the signal. In some embodiments, signal can be removed by any method that reduces the number of signal-generating reporters and/or auxiliary reporters, for example: photobleaching fluorescent reporter molecules using light and/or chemical reactions, chemically cleaving reports from HCR hairpins (e.g., TCEP), chemically cleaving hairpins to fragment HCR amplification polymers, chemically cleaving probes to untether HCR amplification polymers from the target, using an auxiliary strand to dehybridize hairpins from HCR amplification polymers, using an auxiliary strand to dehybridize probes from the target, using chemical denaturants and/or elevated temperature to destabilize HCR amplification polymers, using chemical denaturants and/or elevated temperature to destabilize probes and the target, using enzymes to degrade HCR amplification polymers, using enzymes to degrade probes from the target, using DNases to degrade DNA amplification polymers, using DNases to degrade DNA probes, using DNases to degrade DNA targets, using RNases to degrade RNA targets, using two or more of the above methods or any other method for removing signal from the target at the same time or at different times.

Assay formats. In some embodiments, a signal can be measured in different assay formats including but not limited to: blots, northern blots, western blots, Southern blots, spot blots, paper assays, flow cytometry assays, fluorescent flow cytometry assays, cell sorting assays, fluorescence-activated cell sorting assays, magnetic-activated cell sorting assays, microscopy assays, light microscopy assays, epifluorescence microscopy assays, confocal microscopy assays, light sheet microscopy assays, microarray assays, bead-based assays, mass spectrometry assays, fluorescent microscopy assays, mass spectrometry microscopy assays, mass spectrometry flow cytometry assays, fluorescence assays, chemiluminescence assays, bioluminescence assays, colorimetric assays, electrochemical impedance assays, electrochemical chemiluminescence assays, energy dissipation assays, assays using the human eye, assays using a cell phone camera, gel electrophoresis assays, in situ hybridization (ISH) assays, RNA-ISH assays, DNA-ISH assays, immunohistochemistry (IHC) assays, autoradiography assays, or any assay capable of detecting a signal generated by an HCR amplification polymer.

Sample types. In some embodiments, initiator-labeled probes and/or fractional-initiator probes can be used with HCR amplification hairpins to detect a target in a sample, the target comprising a molecule, a complex, or a collection of proximal molecules or complexes. The target molecule may be contained within a sample, including for example: a bacterium, a zebrafish embryo, a chicken embryo, a mouse embryo, a human biopsy specimen, a human tissue section, an FFPE tissue section, a urine sample, a blood sample, a stool sample, a mouse tissue section, a brain slice, a sea urchin embryo, a nematode larva, a fruit fly embryo, a model organism, a non-model organism, a multi-species mixture of organisms, an environmental sample containing unknown organisms, a consortium of organisms (for example, a mixture of protists and bacteria within the gut of another organism), a termite, a microbiome, a clinical specimen, a diagnostic sample, a sputum sample, a tumor biopsy sample, a research sample, a sample comprising material from a human, a sample comprising material from a pet (for example, a dog, cat, rabbit, lizard, snake, or fish), material from a wild animal (for example, a cheetah, elephant, rhinoceros, or chimpanzee), material from an extinct animal (for example, a woolly mammoth, a dodo, a giant auk, a triceratops, or a passenger pigeon), living cells (for example, bacteria or cultured mammalian cells), or a living organism (for example a living mouse or a living human).

In some embodiments, the target may be free in solution within the sample. For example, the target may be free in solution within: a test tube, a cell, an embryo, an organism, a tissue section, a biological specimen, or other sample.

In some embodiments, the target may be covalently crosslinked or non-covalently bound to one or more capture probes covalently or non-covalently attached to a solid support. For example, bound directly or indirectly to a capture probe covalently linked to a microarray or bead.

In some embodiments, the target may be fixed, covalently crosslinked, or non-covalently bound directly or indirectly to a solid support. For example, the target may be bound, fixed, or covalently cross-linked to a slide, a blot, a membrane, a paper substrate, or any other substrate. The target may be fixed or covalently crosslinked to a cell, embryo, organism, tissue section, biological specimen, or any other sample. The target may be covalently linked within a sample that is fixed and permeabilized, fixed but not permeabilized, or not fixed but permeabilized.

In some embodiments, the target may be free within a living cell, living embryo, living organism, living ecosystem, or consortium of organisms (for example, the microbiome within the gut of a mammal). The target may be associated with but exterior to a cell or organism, or it may be contained within a cell or organism. The target may be covalently crosslinked within a living cell, living embryo, living organism, living ecosystem, or living consortium of organisms. The target may be present within or absent from one or more cell types within the sample. The target may be present within or absent from one or more species of organism within the sample. The target may be present in a sample that contains one or more off-targets that have different degrees of similarity to the target molecule. The target may be present within an expanded sample. The target may be present within a compressed sample. The sample may be expanded prior to detecting the target so as to increase the spatial separation between molecules. The sample may be compressed prior to detecting the target so as to decrease the spatial separation between molecules. The target and/or other molecules may be crosslinked to an expanded sample so as to maintain the relative position between molecules in the sample as the sample expands. The target and/or other molecules may be crosslinked to a gel, matrix, or other reagents introduced to the sample so as to expand the sample while maintaining the relative position and/or orientation of molecules in the sample as the sample expands. The sample may be differentially expanded and/or compressed with different expansion and/or compression factors in different tissues and/or organs within the sample.

Fixing the sample. In some embodiments, a target molecules can be crosslinked to the sample so that they are retained during subsequent steps in an experiment. For example, target molecules can be crosslinked to the sample using chemical reagents (for example, formaldehyde, paraformaldehyde, EDC (1-ethyl-3-(3-dimethylaminopropyl) carbodiimide)).

Permeabilizing the sample. In some embodiments, the sample can be treated to enhance the accessibility of target molecules to HCR probes and amplifiers. For example, the sample (for example, cells, tissue sections, or whole-mount embryos) can be permeabilized using chemical reagents (for example, methanol, ethanol, detergent) or enzymes (for example, proteinase K). Target accessibility can also be enhanced via sample homogenization, microdissection, electroporation, sectioning, heat treatment (for example, Smith J J, Gunasekera T S, Barardi C R, Veal D, Vesey G (2004) J Appl Microbiol 96(2):409-417), and/or microwave treatment (for example, Lan H Y, Mu W, NG Y Y, Nikolic-Paterson D H, & Atkins R C (1996) J Histochem Cytochem 44(3):281-287). Another option is to deliver HCR probes and amplifiers across the cell membrane using chemical transfection reagents.

Sample washes to remove unbound reagents from the sample. In some embodiments, background can be reduced by washing unused imaging reagents from the sample. For example, washes can be used to remove probes, initiator-labeled probes, fractional-initiator probes, HCR amplification hairpins, amplification reagents, label probes, antibodies, and/or other imaging reagents from the sample. Washes can be performed at a temperature using chemical reagents such that imaging reagents that are bound specifically are predominantly not removed (retaining signal) and imaging reagents that are bound non-specifically are predominantly removed (reducing background). For example, wash buffers could include denaturing agents (e.g., formamide, urea), salt buffer (e.g., sodium chloride sodium citrate (SSC), phosphate buffered saline (PBS)), acids (e.g., citric acid), surfactants (e.g., Tween 20, Triton-X, SDS), or blocking agents (e.g., tRNA, salmon sperm DNA, BSA, ficoll, polyvinilypyrolidone, heparin). Wash buffer can be combined with wash temperature (e.g., 25-80° C.) to optimize wash stringency.

### EXEMPLARY EMBODIMENTS

FIG. 6 depicts the detection of a target complex comprising two target molecules with fractional-initiator probes and a proximity probe, wherein the fractional-initiator probes directly bind their targets. Fractional-initiator probe 1 comprising target-binding domain anti-Target 1 binds Target 1, and Fractional-initiator probe 2 comprising target-binding domain anti-Target 2 binds Target 2. With both fractional-initiator probes bound to their targets and in proximity to one another, the proximity probe can bind proximity domains 1 and 2, thereby allowing fractional initiators 1 and 2 to colocalize and form a full initiator capable of initiating HCR.

FIG. 7 depicts the detection of a target complex comprising two target molecules with fractional-initiator probes and a proximity probe, wherein the fractional-initiator probes indirectly bind their targets. Primary probe anti-Target 1 binds Target 1 and primary probe anti-Target 2 binds Target 2. Fractional-initiator probe 1 comprising target-binding domain anti-anti-Target 1 binds to primary probe anti-Target 1 and Fractional-initiator probe 2 comprising target-binding domain anti-anti-Target 2 binds to primary probe anti-Target 2. With both fractional-initiator probes bound to their targets and in proximity to one another, the proximity probe can bind proximity domains 1 and 2, thereby allowing fractional initiators 1 and 2 to colocalize and form a full initiator capable of initiating HCR.

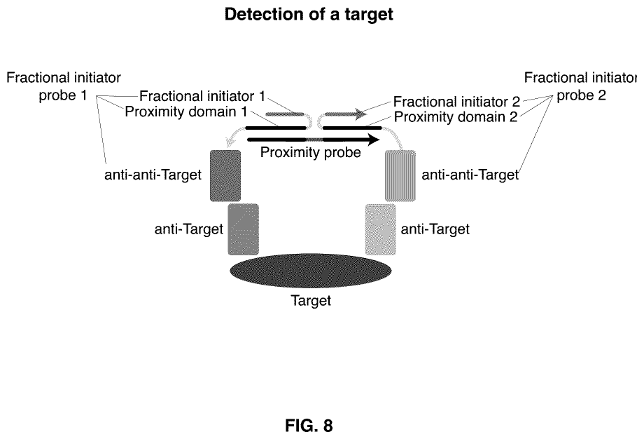

FIG. 8 depicts the detection of a target molecule with two fractional-initiator probes and a proximity probe, wherein the fractional-initiator probes indirectly bind different sites on the target. Two separate sites on the target are bound by anti-target primary probes. Fractional-initiator probe 1 binds one anti-Target primary probe with its anti-anti-Target domain, and Fractional-initiator probe 2 binds the other anti-Target primary probe with its anti-anti-Target domain. With both fractional-initiator probes bound to their targets and in proximity to one another, the proximity probe can bind proximity domains 1 and 2, thereby allowing fractional initiators 1 and 2 to colocalize and form a full initiator capable of initiating HCR.

FIG. 9 depicts detection of a first target molecule and a second target molecule with two fractional-initiator probes and a proximity probe, wherein the first and second target molecules are not in a complex with each other but are in a complex with a third target molecule. Primary probe anti-Target 1 binds Target 1 and primary probe anti-Target 2 binds Target 2, wherein Targets 1 and 2 are not bound to each other directly, but both are bound to Target 3. Fractional-initiator probe 1 binds anti-Target 1 with its anti-anti-Target 1 domain, and Fractional-initiator probe 2 binds anti-Target 2 with its anti-anti-Target 2 domain. With both fractional-initiator probes bound to their targets and in proximity to one another, the proximity probe can bind proximity domains 1 and 2, thereby allowing fractional initiators 1 and 2 to colocalize and form a full initiator capable of initiating HCR.

FIG. 10 depicts detection of a first target and a second target with two fractional-initiator probes and a proximity probe, wherein the first and second targets are proximal but not in a complex with each other. Targets 1 and 2 of the target complex are bound by anti-Targets 1 and 2 respectively, wherein Targets 1 and 2 are not bound to each other directly. Fractional-initiator probe 1 binds anti-Target 1 with its anti-anti-Target 1 domain, and Fractional-initiator probe 2 binds anti-Target 2 with its anti-anti-Target 2 domain. With both fractional-initiator probes bound to their targets and in proximity to one another, the proximity probe can bind proximity domains 1 and 2, thereby allowing fractional initiators 1 and 2 to colocalize and form a full initiator capable of initiating HCR.

FIG. 11 depicts detection of a target with two fractional-initiator probes and a proximity probe, wherein the fractional-initiator probes bind the target indirectly via binding to different sites on a primary probe that directly binds the target. Primary probe anti-Target binds the Target. Fractional-initiator probe 1 and fractional-initiator probe 2 bind the different sites on the anti-Target primary probe with their respective anti-anti-Target domains. With both fractional-initiator probes bound to their targets and in proximity to one another, the proximity probe can bind proximity domains 1 and 2, thereby allowing fractional initiators 1 and 2 to colocalize and form a full initiator capable of initiating HCR.

FIG. 12 depicts detection of three targets using fractional-initiator probes and a proximity probe, wherein the three targets are in a complex together. Targets 1, 2, and 3 of the target complex are bound by primary probes anti-Targets 1, 2, and 3 respectively. Fractional-initiator probe 1 binds anti-Target 1 with its anti-anti-Target 1 domain, fractional-initiator probe 2 binds anti-Target 2 with its anti-anti-Target 2 domain, and fractional-initiator probe 3 binds anti-Target 3 with anti-anti-Target 3 domain. With all fractional-initiator probes bound to their targets and in proximity to one another, the proximity probe can bind proximity domains 1, 2, and 3, thereby allowing fractional initiators 1, 2, and 3 to colocalize and form a full initiator capable of initiating HCR.

FIG. 13 depicts detection of three targets using fractional-initiator probes and two proximity probes, wherein the three targets are in a complex together. Targets 1, 2, and 3 of the target complex are bound by primary probes anti-Targets 1, 2, and 3 respectively. Fractional-initiator probe 1 binds anti-Target 1 with its anti-anti-Target 1 domain, fractional-initiator probe 2 binds anti-Target 2 with its anti-anti-Target 2 domain, and fractional-initiator probe 3 binds anti-Target 3 with its anti-anti-Target 3 domain. With all fractional-initiator probes bound to their targets and in proximity to one another, proximity probe 1 can bind proximity domains 1 and 2, and proximity probe 2 can bind proximity domains 2 and 3, thereby allowing fractional initiators 1, 2, and 3 to colocalize and form a full initiator capable of initiating HCR.

FIG. 14 depicts detection of two protein targets using fractional-initiator probes and a proximity probe, wherein the targets are in a protein:protein complex. Proteins 1 and 2 of the target complex are bound by primary antibody probes anti-protein 1 and anti-protein 2 respectively. Fractional-initiator probe 1 binds anti-Protein 1 with its secondary antibody domain, and Fractional-initiator probe 2 binds anti-Protein 2 with its secondary antibody domain. With both fractional-initiator probes bound to their targets and in proximity to one another, the proximity probe can bind proximity domains 1 and 2, thereby allowing fractional initiators 1 and 2 to colocalize and form a full initiator capable of initiating HCR.

### Additional Embodiments

Any of the embodiments, compositions, and/or methods provided herein can be employed with, or in the alternative form of, any of the following. Thus, for example, the above noted compositions and/or methods can employ any of the compositions or methods noted below. Similarly, the above noted compositions and/or methods should be understood to also provide methods employing the methods below or as being part of the methods noted below.

Similarly, the embodiments and/or methods provided herein should also be understood to provide embodiments involved in the method, e.g., compositions, components of the method, kits, etc. In some embodiments, any of the ingredients in one or more of the methods and/or steps provided herein can be provided as a kit including one or more of the noted ingredients (and optionally the target or target sequence or sample).

### Compositions

Some embodiments of compositions are outlined in FIGS. 46A and 46B, as well as other figures provided herein. In some embodiments, a composition is provided that includes a first fractional-initiator probe comprising: a first target-binding domain configured to bind directly or indirectly to a first target, a first proximity domain, a first fractional initiator, a second fractional-initiator probe comprising: a second target-binding domain configured to bind directly or indirectly to a second target, a second proximity domain, a second fractional initiator, a proximity probe configured to bind the first proximity domain and the second proximity domain; and an HCR amplifier comprising two or more HCR hairpins. Optionally, the composition can comprise additional fractional-initiator probes or proximity probes.

In some embodiments, the first and second fractional initiators together form a full HCR initiator when bound by a proximity probe, where the full HCR initiator is capable of binding to and opening the first HCR hairpin. In some embodiments, the first fractional-initiator probe further comprises a first target-binding domain and the second fractional-initiator probe further comprises a second target-binding domain, wherein the first target-binding domain is configured to bind to a first target and the second target binding domain is configured to bind to a second target. In some embodiments, these target-binding domains are positioned in proximity to one another following binding of their respective targets, such that when both fractional-initiator probes are bound to both target domains, they can be bound by the proximity probe at their respective proximity domains, and the first and second fractional initiators (within the fractional-initiator probes) colocalized to form a full HCR initiator capable of binding to and opening the first HCR hairpin.

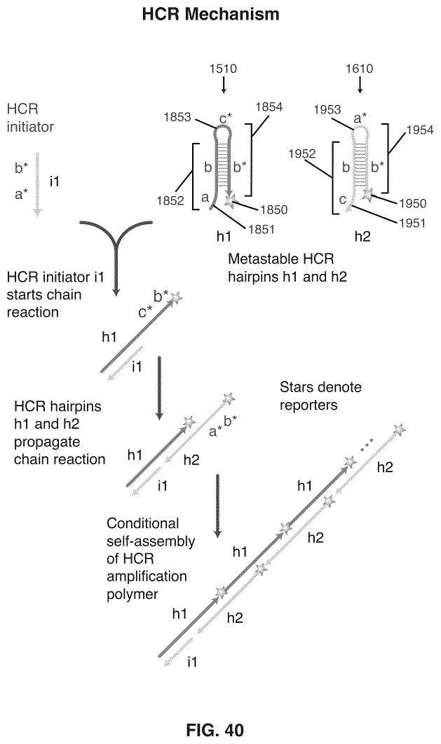

Some embodiments of compositions of HCR monomers are outlined in FIGS. 39 and 40, as well as other figures provided herein.

In some embodiments, a composition is provided that includes a first HCR hairpin (1510), comprising: a) a first input domain (1852), comprising a first toehold (1851) and a first stem section (1755), b) a first output domain (1854), comprising a first loop (1853) and a complement to the first stem section (1756), and optionally c) one or more reporters (1850). In some embodiments, the composition further includes a second HCR hairpin (1610), comprising: a) a second input domain (1952), comprising a second toehold (1951) and a second stem section (1855), b) a second output domain (1954), comprising a second loop (1953) and a complement to the second stem section (1856), and optionally c) one or more reporters (1950).

In some embodiments, the first toehold (1851) is complementary to the second loop (1953). In some embodiments, the second toehold (1951) is complementary to the first loop (1853). In some embodiments, this circularity allows HCR polymerization to occur in the presence of an initiator that is able to bind to the first input domain, as described herein. In some embodiments, the first toehold is not 100% complementary to the second loop but is sufficient to allow hybridization.

### Methods

In some embodiments, methods are provided comprising: A) providing: a sample containing a first target and a second target; a first fractional-initiator probe comprising: a first target-binding domain configured to bind directly or indirectly to the first target, a first proximity domain, a first fractional initiator; a second fractional-initiator probe comprising: a second target-binding domain configured to bind directly or indirectly to the second target, a second proximity domain, a second fractional initiator; a proximity probe configured to bind the first proximity domain and the second proximity domain; an HCR amplifier comprising two or more HCR hairpins; B) incubating the first fractional-initiator probe and the second fractional-initiator probe in the sample to allow for binding; C) incubating the proximity probe in the sample to allow for binding; D) incubating the HCR amplifier in the sample; E) and detecting a signal. In some embodiments, the first fractional-initiator probe is bound to the first target and the second fractional-initiator probe is bound to the second target, and when the first and second target are bound to each other and/or are proximal, then the proximity probe binds to the first proximity domain and the second proximity domain to colocalize a full initiator. In some embodiments, the colocalized full initiator is used to trigger hybridization chain reaction (HCR) signal amplification.

In some embodiments, the method further comprises providing, a third fractional-initiator probe comprising: a third target-binding domain configured to bind directly or indirectly to the third target, a third proximity domain, and a third fractional initiator. In some embodiments, the proximity probe is configured to bind the first proximity domain the second proximity domain, and the third proximity domain. In some embodiments, a wash step is performed to remove unbound fractional-initiator probes following B). In some embodiments, a wash step is performed to remove unbound proximity probes following C). In some embodiments, a wash step is performed to remove unbound HCR hairpins following D). In some embodiments, the signal is removed following E). In some embodiments, any of the above steps are repeated to detect a signal for the same or different targets.

In some embodiments, the signal is detected by a fluorescence microscope, a fluorescence scanner, a camera, a mobile phone camera, a mass spectrometer, a mass spectrometry microscope, or a radioactive scanner. In some embodiments, the method further comprises providing a helper probe to maximize signal generation. In some embodiments, a helper probe does not comprise a fractional initiator. In some embodiments, a helper probe does not comprise a proximity domain. In some embodiments, binding of one or more helper probes to the target increases target accessibility to facilitate the binding of one or more fractional-initiator probes to the target (e.g., see FIG. 30D). In some embodiments, the first target is a protein, a nucleic acid, a molecule, or a combination thereof, and wherein the second target is a protein, a nucleic acid, a molecule, or a combination thereof. In some embodiments, the first target and the second target are bound to each other. In some embodiments, the first target and the second target are the same molecule. In some embodiments, the proximity probe contains one or more clamp domains configured to bind to some or all of the first fractional initiator and/or the second fractional initiator if one target is not proximal. In some embodiments, the first fractional-initiator probe and/or the second fractional-initiator probe comprises an antibody, a nanobody, and/or an oligonucleotide. In some embodiments, the colocalized full initiator is used to mediate generation of a signal directly or indirectly. In some embodiments, the colocalized full initiator triggers self-assembly of metastable fluorophore-labeled HCR hairpins into a tethered fluorescent amplification polymer to generate an amplified signal at the site of the targets. In some embodiments, the colocalized full initiator is utilized to detect one or more protein:protein complexes, RNA:protein complexes, RNA:RNA complexes, DNA:protein complexes, DNA:protein:protein complexes, or a complex of three or more RNA, DNA, and/or protein or other molecules in a sample. In some embodiments, fractional-initiator probes that bind nonspecifically do not colocalize a full initiator and do not initiate HCR. In some embodiments. In some embodiments, three cooperative probe junctions comprise the oligonucleotide sequences displayed in Table 1, where each cooperative probe junction comprises a fractional-initiator probe P1 comprising a fractional initiator and a proximity domain, a fractional-initiator probe P2 comprising a fractional initiator and a proximity domain, and a proximity probe. In some embodiments, the signal generation comprises a fluorophore, a chromophore, a luminophore, a phosphor, a FRET pair, a member of a FRET pair, a quencher, a fluorophore/quencher pair, a rare earth element or compound, a radioactive molecule, a nucleotide, an amino acid, an oligonucleotide, DNA, RNA, 2′Ome-RNA, a chemically modified nucleic acid, a synthetic nucleic acid analog, a chemically modified protein, a synthetic protein analog, a peptide, a binding substrate, a carbon atom, a chemical linker, a magnetic molecule, carbon black (CB), carbon nanotubes, magnetized carbon nanotubes, gold nanoparticles (AuNP), gold nanoshells, gold nanorods, silver-shelled gold nanoparticles, latex, magnetic nanoparticles, silica nanoparticles, a fluorophore, fluorophore-loaded nanoparticles, dye-loaded nanoparticles, an enzyme, any combination thereof. In some embodiments, the method further comprises providing a hapten, a ligand, an oligonucleotide, digoxigenin (DIG), fluorescein isothiocyanate (FITC), a fluorophore, biotin, dinitrophenol, aniline, or an enzyme utilized to facilitate generation of a signal.

In some embodiments, the target in a sample undergoes blocking for approximately one hour before fractional-initiator probes or anti-target molecules are incubated with the target. In some embodiments, one or more anti-target molecules are incubated with the target in a sample before two or more fractional-initiator probes are incubated with the target. In some embodiments, the one or more anti-target molecules comprise unlabeled primary antibody probes. In some embodiments, the two or more fractional-initiator probes are incubated with the sample following incubation with the one or more anti-target molecules. In some embodiments, the two or more fractional-initiator probes comprise fractional-initiator secondary antibody probes. In some embodiments, the two or more fractional-initiator probes are incubated with the sample overnight, or approximately 17 hours. In some embodiments, one or more proximity probes are added to the sample following overnight incubation with the two or more fractional-initiator probes. In some embodiments, the one or more proximity probes are incubated with the sample for approximately four hours. In some embodiments, HCR amplifiers are added to the sample following incubation with the proximity probes. In some embodiments, the HCR amplifiers are incubated with the sample overnight, or approximately 20 hours. In some embodiments, a wash step is performed prior to detection.

In some embodiments, the target in a sample undergoes blocking for approximately one hour before one or more anti-target molecules are incubated with the target. In some embodiments, some or all of three types of targets are detected in the same sample at the same time in a multiplex experiment (for example, FIG. 47): 1) targets that are individual protein molecules, 2) targets that are two proteins forming a complex or in proximity, and 3) targets that are individual RNA molecules). In some embodiments, in a protein detection phase, one or more unlabeled primary anti-target probes are incubated in the sample, optionally followed by one or more wash steps, and then one or more initiator-labeled secondary probes and two or more fractional-initiator secondary probes are incubated in the sample, optionally followed by one or more wash steps. In some embodiments, the total incubation time for the primary probes plus the total incubation time for the secondary probes is approximately 4, or 8, or 16, or 24 hours. In some embodiments, the primary probes comprise primary antibodies or nanobodies and the secondary probes comprise secondary antibodies or nanobodies. In some embodiments, in a proximity stage, one or more proximity probes are incubated in the sample, optionally followed by one or more wash steps. In some embodiments, the one or more proximity probes are incubated with the sample for approximately 0.5, or 1, or 2, or 4, or 8, or 16, or 24 hours. In some embodiments, in an RNA detection stage, two or more fractional-initiator DNA probes are incubated with the sample, optionally followed by one or more wash steps. In some embodiments, the DNA probes are incubated in the sample for approximately 1, or 2, or 4, or 8, or 16, or 20, or 24 hours. In some embodiments, in an amplification stage, HCR amplifiers are added to the sample, optionally followed by one or more wash steps. In some embodiments, the HCR amplifiers are incubated with the sample for approximately 0.5, or 1, or 2, or 10, or 16, or 24 hours. In some embodiments, after detecting a signal for one or more targets, the signal is removed from the sample and one or more of the above steps is repeated to detect a signal for one or more new targets.

### Examples

### Example 1—Imaging a Protein:Protein Target Complex

FIG. 15 depicts imaging a protein:protein target complex in fixed adherent A-431 human cells using a pair of fractional-initiator probes, a proximity probe, and an HCR amplifier. For each protein:protein complex, background was assessed by technical controls in which the antibodies corresponding to one protein of the complex were omitted, simulating a scenario in which the two proteins are not in a complex with one another. For each of three protein:protein target complexes, a high signal-to-background ratio was obtained: 170±10 (SC35:SON), 50±10 (β-catenin:E-cadherin), and 140±10 (α-tubulin:β-tubulin) (standard error of the mean, representative regions within N=3 cells).

FIG. 16 depicts imaging the β-catenin:E-cadherin target complex in a highly autofluorescent FFPE human breast tissue section (5-μm thickness) using a pair of fractional-initiator probes, a proximity probe, and an HCR amplifier. Background was assessed by fluorescence in a region of the sample in which there is low/no abundance of the protein:protein complex. A high signal-to-background ratio was obtained (70±20) (standard error of the mean, N=3 representative regions).

### Example 2—Multiplexed Imaging of Protein:Protein Target Complexes

FIG. 17 depicts 3-plex imaging of protein:protein complexes in fixed adherent A-431 human cells utilizing a different pair of fractional-initiator probes, orthogonal HCR amplifier, and proximity probe for each target complex. The three orthogonal HCR amplifiers carried spectrally distinct fluorophores to enable multiplexed imaging. Imaging revealed fluorescent signal from the nuclear SC35:SON complex, membranous β-catenin:E-cadherin complex, and cytoskeletal α-tubulin:β-tubulin complex.

### Example 3—Simultaneous Multiplexed Imaging of a Protein:Protein Complex, a Protein Target, and an RNA Target

FIG. 18 depicts simultaneous HCR imaging of a protein:protein complex, a protein target, and an RNA target in fixed adherent A-431 human cells. The reagents and protocol are depicted in FIG. 47. The protein target was detected with an unlabeled primary antibody probe which was in turn bound by an initiator-labeled secondary antibody probe. The RNA target was detected using a probe set comprising two pairs of fractional-initiator DNA probes. The protein:protein target complex was detected with two unlabeled primary antibody probes that were in turn bound by fractional-initiator secondary antibody probes that were in turn bound by a proximity probe to colocalize a full HCR initiator. The three target types (protein:protein, protein, RNA) each triggered orthogonal HCR amplifiers carrying spectrally distinct fluorophores. Amplified HCR signal revealed membranous β-catenin:E-cadherin complex, cytoskeletal β-tubulin protein, and nuclear U6 RNA.

### Example 4—Quantitative HCR (qHCR) Imaging of a Protein:Protein Complex

Quantitative protein:protein complex imaging is demonstrated via the 2-channel redundant detection experiment of FIG. 19A. FIG. 19B depicts redundant detection of the membranous β-catenin:E-cadherin protein:protein complex in fixed adherent A-431 human cells. FIG. 19C displays a 2-channel scatter plot of voxel intensities for subcellular 2×2×0.8 μm voxels in the rectangular region of FIG. 19B, revealing a tight linear distribution (Pearson correlation coefficient, r) corresponding to accurate and precision relative quantitation. Accuracy corresponds to linearity with zero intercept and precision corresponds to scatter around the line.

### Example 5—Imaging Protein:Protein Complexes with High Signal-to-Background

FIG. 45 demonstrates the performance of HCR protein:protein imaging by comparing the fluorescence intensity between two pairs of biological sample types using the same imaging settings for both sample types. Positive samples contain the protein:protein complex of interest; negative samples did not contain the protein:protein complex of interest. For each pair of sample types, signal-to-background was calculated using the positive sample type to estimate signal plus background and the negative sample type to estimate background.

The β-catenin:E-cadherin target complex was imaged using A-431 adherent human cells as the positive sample and HeLa adherent human cells as the negative sample. While A-431 cells formed the β-catenin:E-cadherin complex at the cell membrane of intercellular junctions,⁶³ HeLa cells express N-cadherin rather than E-cadherin^{64,65} and therefore lacked the β-catenin:E-cadherin complex. A-431 cells (FIG. 45A) displayed strong signal at intercellular junctions and HeLa cells displayed no visible staining (FIG. 45B), with a signal-to-background ratio of 26±4 between the two cell lines (mean±SEM for representative regions of N=3 replicate wells on a slide).

The β-catenin:E-cadherin target complex was detected in highly autofluorescent FFPE human breast tissue sections. The β-catenin:E-cadherin complex is robustly formed in normal breast epithelial cells, but the expression of and interaction between the β-catenin and E-cadherin proteins is interrupted when breast epithelial cells become cancerous in the invasive lobular carcinoma disease process.^{66,67} Paired normal and invasive lobular carcinoma FFPE breast tissue sections from the same patient were evaluated for the β-catenin:E-cadherin target complex, yielding strong HCR signal in normal breast tissue (FIG. 45C; positive sample) and no visible staining in cancerous tissue (FIG. 45D; negative sample), with a signal-to-background ratio of 30±3 between the two tissue types (mean±SEM for representative regions of N=3 replicate sections).

### Example 6—Sequences for Cooperative Probe Junctions

Table 1 displays sequences for three cooperative probe junctions. Each cooperative probe junction comprises: a) P1 oligonucleotide comprising a fractional initiator (sequence underlined) and a proximity domain (sequence in bold), b) a P2 oligonucleotide comprising a fractional initiator (sequence underlined) and a proximity domain (sequence in bold), c) a proximity probe comprising the binding sites for the P1 and P2 proximity domains (sequence in bold). Note that a fractional-initiator probe P1 comprises the P1 oligonucleotide and that a fractional-initiator probe P2 comprises the P2 oligonucleotide (for example, see the schematic of FIG. 46A).

The two 1-plex imaging studies of FIGS. 45A-45B and 45C-45D each employ a fractional-initiator antibody probe pair (P1 and P2) and a proximity probe that form cooperative probe junction J1 (oligonucleotide sequences in Table 1).

The 3-plex imaging study of FIG. 17 employs fractional-initiator probe pairs and proximity probes that form a different cooperative probe junction for each target complex: junction J1 for β-catenin:E-cadherin, junction J2 for α-tubulin:β-tubulin, and junction J3 for SC35:SON.

| | Oligo- | Length | |
|---|---|---|---|
| Junction | nucleotide | (nt) | Sequence (5′ to 3′) |
| J1 | P1 probe | 49 | GAGGAGGGCAGCAAACGGTCGCCCATGTGTACCCGAAATTCAA |
| | | | GTCAGC (SEQ ID NO: 1) |
| | P2 probe | 49 | TTCCTGATTCTATTACTTGCCTGATCCCTATGAAGAGTCTTCC |
| | | | TTTACG (SEQ ID NO: 2) |
| | Proximity | 50 | CTTGAATTTCGGGTACACATGGGCTAAGGGATCAGGCAAGTAA |
| | probe | | TAGAATC (SEQ ID NO: 3) |
| J2 | P1 probe | 49 | GCAAACTAAACATCCCACAAGGTGAAAGCTGGTACGAATAAGA |
| | | | CTACGC (SEQ ID NO: 4) |
| | P2 probe | 49 | CTGCACCGGTATATGTTCTGAAGGTGATGCATCCAACTCTAAC |
| | | | TAAATC (SEQ ID NO: 5) |
| | Proximity | 50 | GTCTTATTCGTACCAGCTTTCACCACCATCACCTTCAGAACAT |
| | probe | | ATACCGG (SEQ ID NO: 6) |
| J3 | Pl probe | 49 | CTAACAATCTAAACATACTCGAGGGTGCGGTCTATTCTATTTC |
| | | | CAACGT (SEQ ID NO: 7) |
| | P2 probe | 49 | TATTGCGTGTTAGGTGAGTTTGAGATTTGTACACGCCCAAGAA |
| | | | CATAAA (SEQ ID NO: 8) |
| | Proximity | 50 | GGAAATAGAATAGACCGCACCCTCACCAAATCTCAAACTCACC |
| | probe | | TAACACG (SEQ ID NO: 9) |

### Table 1. Oligonucleotide Sequences for Cooperative Probe Junctions.

### Additional Arrangements

Arrangement 1: A composition comprising: a first fractional-initiator probe comprising: a first target-binding domain configured to bind directly or indirectly to a first target, a first proximity domain, a first fractional initiator, a second fractional-initiator probe comprising: a second target-binding domain configured to bind directly or indirectly to a second target, a second proximity domain, a second fractional initiator, a proximity probe configured to bind the first proximity domain and the second proximity domain; a hybridization chain reaction (HCR) amplifier comprising two or more HCR hairpin monomers at least one of which comprises a reporter; and wherein when the first fractional-initiator probe is bound to the first target and the second fractional-initiator probe is bound to the second target; and the first and the second target are bound to each other and/or are proximal, the proximity probe is able to bind the first proximity domain and the second proximity domain to colocalize a full initiator comprising the first fractional initiator and the second fractional initiator, and wherein the colocalized full initiator is configured to initiate HCR signal amplification whereupon the HCR hairpin monomers self-assemble into a tethered HCR amplification polymer thereby generating a signal.

Arrangement 2: The composition of Arrangement 1, wherein at least one of the HCR hairpin monomers comprises an input domain and wherein the first fractional initiator and second fractional initiator together form a full initiator configured to hybridize to the input domain.

Arrangement 3: The composition of any one of Arrangements 1-2, further comprising: a third fractional-initiator probe comprising: a third target-binding domain configured to bind directly or indirectly to a third target, a third proximity domain, a third fractional initiator; and a proximity probe further configured to bind the third proximity domain.

Arrangement 4: The composition of any one of Arrangements 1-3, wherein the first target is a protein, a nucleic acid, or a combination thereof, and wherein the second target is a protein, a nucleic acid, or a combination thereof.

Arrangement 5: The composition of Arrangement 4, wherein the first target and the second target are bound to each other.

Arrangement 6: The composition of Arrangement 4, wherein the first target and the second target are the same molecule.

Arrangement 7: The composition of any one of Arrangements 1-6, wherein the proximity probe contains one or more clamp domains configured to bind to some or all of the first fractional initiator and/or the second fractional initiator if one target is not proximal.

Arrangement 8: The composition of any one of Arrangements 1-7, where the first fractional-initiator probe and/or the second fractional-initiator probe comprises an antibody, a nanobody, and/or an oligonucleotide.

Arrangement 9: The composition of any one of Arrangements 1-8, wherein the colocalized full initiator is used to mediate generation of a signal directly or indirectly.

Arrangement 10: The composition of any one of Arrangements 1-9, wherein the colocalized full initiator triggers self-assembly of metastable fluorophore-labeled HCR hairpins into a tethered fluorescent amplification polymer to generate an amplified signal at the site of the targets.

Arrangement 11: The composition of any one of Arrangements 1-10, wherein the first target and second target are selected from protein, RNA, DNA, or other nucleic acids.

Arrangement 12: The composition of any one of Arrangements 1-11, wherein the first target and second target form a complex.

Arrangement 13: The composition of Arrangement 12, wherein the composition comprises additional fractional initiators for each N target complex to be detected in a sample.

Arrangement 14: The composition of Arrangement 13, wherein the N target complexes are each detected in the same sample using a different pair of fractional-initiator probes, proximity probe, and HCR amplifier for each target complex.

Arrangement 15: The composition of any one of Arrangements 1-14, wherein the sequence of the first fractional initiator is selected from SEQ ID NO: 1, SEQ ID NO: 4, and SEQ ID NO: 7; the sequence of the second fractional initiator is selected from SEQ ID NO: 2, SEQ ID NO: 5, and SEQ ID NO: 8; and the sequence of the proximity probe is selected from SEQ ID NO: 3, SEQ ID NO: 6, and SEQ ID NO: 9.

Arrangement 16: The composition of any one of Arrangements 1-15, wherein the at least one reporter comprises a fluorophore, a chromophore, a luminophore, a phosphor, a FRET pair, a member of a FRET pair, a quencher, a fluorophore/quencher pair, a rare earth element or compound, a radioactive molecule, a nucleotide, an amino acid, an oligonucleotide, DNA, RNA, 2′Ome-RNA, a chemically modified nucleic acid, a synthetic nucleic acid analog, a chemically modified protein, a synthetic protein analog, a peptide, a binding substrate, a carbon atom, a chemical linker, a magnetic molecule, carbon black (CB), carbon nanotubes, magnetized carbon nanotubes, gold nanoparticles (AuNP), gold nanoshells, gold nanorods, silver-shelled gold nanoparticles, latex, magnetic nanoparticles, silica nanoparticles, fluorophore-loaded nanoparticles, dye-loaded nanoparticles, an enzyme, or any combination thereof.

Arrangement 17: The composition of any one of Arrangements 1-16, wherein the at least one reporter mediates generation of the signal directly or indirectly.

Arrangement 18: The composition of any one of Arrangements 1-17, wherein the at least one reporter comprises a hapten, a ligand, an oligonucleotide, digoxigenin (DIG), fluorescein isothiocyanate (FITC), a fluorophore, biotin, dinitrophenol, aniline, or an enzyme utilized to facilitate generation of the signal.

Arrangement 19: The composition of any one of Arrangements 1-18, wherein the HCR amplification polymer mediates catalytic reporter deposition (CARD).

Arrangement 20: A method comprising: providing: A sample containing a first target and a second target; A first fractional-initiator probe comprising: a first target-binding domain configured to bind directly or indirectly to the first target, a first proximity domain, a first fractional initiator; A second fractional-initiator probe comprising: a second target-binding domain configured to bind directly or indirectly to the second target, a second proximity domain, a second fractional initiator; A proximity probe configured to bind the first proximity domain and the second proximity domain; A hybridization chain reaction (HCR) amplifier comprising two or more HCR hairpin monomers at least one of which comprises a reporter; adding the first fractional-initiator probe and the second fractional-initiator probe to the sample; adding the proximity probe to the sample; adding the HCR amplifier to the sample; detecting a signal indicating the presence of an HCR polymer; wherein when the first fractional-initiator probe is bound to the first target and the second fractional-initiator probe is bound to the second target, and when the first and second target are bound to each other or are proximal, then the proximity probe binds to the first proximity domain and the second proximity domain to colocalize a full initiator; and wherein the colocalized full initiator initiates polymerization of the HCR monomers thereby generating a signal.

Arrangement 21: The method of Arrangement 20, wherein the method further comprises providing, a third fractional-initiator probe comprising: a third target-binding domain configured to bind directly or indirectly to the third target, a third proximity domain, and a third fractional initiator.

Arrangement 22: The method of Arrangement 21, wherein the proximity probe is configured to bind the first proximity domain the second proximity domain, and the third proximity domain.

Arrangement 23: The method of any one of Arrangements 20-22, wherein a wash step is performed to remove unbound fractional-initiator probes following b and before c.

Arrangement 24: The method of any one of Arrangements 20-23, wherein a wash step is performed to remove unbound proximity probes following c and before d.

Arrangement 25: The method of any one of Arrangements 20-24, wherein a wash step is performed to remove unbound HCR hairpins following d.

Arrangement 26: The method of any one of Arrangements 20-25, wherein the signal is removed following e.

Arrangement 27: The method of Arrangement 26, wherein any of the above steps are repeated to detect a signal for the same or different targets.

Arrangement 28: The method of any one of Arrangements 20-27, wherein the signal is detected by a fluorescence microscope, a fluorescence scanner, a camera, a mobile phone camera, a mass spectrometer, a mass spectrometry microscope, or a radioactive scanner.

Arrangement 29: The method of any one of Arrangements 20-28, further comprising providing a helper probe to maximize signal generation.

Arrangement 30: The method of any one of Arrangements 20-29, wherein the first target is a protein, a nucleic acid, or a combination thereof, and wherein the second target is a protein, a nucleic acid, or a combination thereof.

Arrangement 31: The method of Arrangement 30, wherein the first target and the second target are bound to each other.

Arrangement 32: The method of Arrangement 30, wherein the first target and the second target are the same molecule.

Arrangement 33: The method of any one of Arrangements 20-32, wherein the proximity probe contains one or more clamp domains configured to bind to some or all of the first fractional initiator and/or the second fractional initiator if one target is not proximal.

Arrangement 34: The method of any one of Arrangements 20-33, where the first fractional-initiator probe and/or the second fractional-initiator probe comprises a target binding region comprising an antibody, a nanobody, and/or an oligonucleotide.

Arrangement 35: The method of any one of Arrangements 20-34, wherein the colocalized full initiator is used to mediate generation of a signal directly or indirectly.

Arrangement 36: The method of any one of Arrangements 20-34, wherein the colocalized full initiator triggers self-assembly of metastable fluorophore-labeled HCR hairpins into a tethered fluorescent amplification polymer to generate an amplified signal at the site of the proximal targets.

Arrangement 37: The method of any one of Arrangements 20-36, wherein the colocalized full initiator is utilized to detect one or more protein:protein complexes, RNA:protein complexes, RNA:RNA complexes, DNA:protein complexes, DNA:protein:protein complexes, or a complex of three or more RNA, DNA, and/or protein or other molecules in a sample.

Arrangement 38: The method of any one of Arrangements 20-37, wherein fractional-initiator probes that bind nonspecifically do not colocalize a full initiator and do not initiate HCR.

Arrangement 39: The method of any one of Arrangements 20-38, wherein N target complexes are each detected in the same sample using a different pair of fractional-initiator probes, proximity probe, and HCR amplifier for each target complex.

Arrangement 40: The method of any one of Arrangements 20-39, wherein the sequence of the first fractional initiator is selected from SEQ ID NO: 1, SEQ ID NO: 4, and SEQ ID NO: 7; the sequence of the second fractional initiator is selected from SEQ ID NO: 2, SEQ ID NO: 5, and SEQ ID NO: 8; and the sequence of the proximity probe is selected from SEQ ID NO: 3, SEQ ID NO: 6, and SEQ ID NO: 9.

Arrangement 41: The method of any one of Arrangements 20-40, wherein the at least one reporter comprises a fluorophore, a chromophore, a luminophore, a phosphor, a FRET pair, a member of a FRET pair, a quencher, a fluorophore/quencher pair, a rare earth element or compound, a radioactive molecule, a nucleotide, an amino acid, an oligonucleotide, DNA, RNA, 2′Ome-RNA, a chemically modified nucleic acid, a synthetic nucleic acid analog, a chemically modified protein, a synthetic protein analog, a peptide, a binding substrate, a carbon atom, a chemical linker, a magnetic molecule, carbon black (CB), carbon nanotubes, magnetized carbon nanotubes, gold nanoparticles (AuNP), gold nanoshells, gold nanorods, silver-shelled gold nanoparticles, latex, magnetic nanoparticles, silica nanoparticles, a fluorophore, fluorophore-loaded nanoparticles, dye-loaded nanoparticles, an enzyme, any combination thereof.

Arrangement 42: The method of any one of Arrangements 20-41, wherein the at least one reporter mediates generation of the signal directly or indirectly.

Arrangement 43: The method of any one of Arrangements 20-42, wherein the at least one reporter comprises a hapten, a ligand, an oligonucleotide, digoxigenin (DIG), fluorescein isothiocyanate (FITC), a fluorophore, biotin, dinitrophenol, aniline, or an enzyme utilized to facilitate generation of the signal.

Arrangement 44: The method of any one of Arrangements 20-43, wherein the HCR amplification polymer mediates catalytic reporter deposition (CARD).

Although the foregoing invention has been described in terms of certain preferred embodiments, other embodiments will be apparent to those of ordinary skill in the art. Additionally, other combinations, omissions, substitutions and modification will be apparent to the skilled artisan, in view of the disclosure herein. Accordingly, the present invention is not intended to be limited by the recitation of the preferred embodiments, but is instead to be defined by reference to the appended claims.

Any of the embodiments, compositions, and/or methods provided herein can be employed with, or in the alternative form of, any of the following. Thus, for example, the above noted compositions and/or methods can employ any of the compositions or methods noted below. Similarly, the above noted compositions and/or methods should be understood to also provide methods employing the methods below or as being part of the methods noted below.

Similarly, the embodiments and/or methods provided herein should also be understood to provide embodiments involved in the method, e.g., compositions, components of the method, kits, etc. In some embodiments, any of the ingredients in one or more of the methods and/or steps provided herein can be provided as a kit including one or more of the noted ingredients (and optionally the target or target sequence or sample).

The section headings used herein are for organizational purposes only and are not to be construed as limiting the described subject matter in any way. All literature and similar materials cited in this application, including but not limited to, patents, patent applications, articles, books, treatises, and internet web pages are expressly incorporated by reference in their entirety for any purpose. When definitions of terms in incorporated references appear to differ from the definitions provided in the present teachings, the definition provided in the present teachings shall control. It will be appreciated that there is an implied “about” prior to the temperatures, concentrations, times, etc. discussed in the present teachings, such that slight and insubstantial deviations are within the scope of the present teachings herein. In this application, the use of the singular includes the plural unless specifically stated otherwise. Also, the use of “comprise”, “comprises”, “comprising”, “contain”, “contains”, “containing”, “include”, “includes”, and “including” are not intended to be limiting. It is to be understood that both the foregoing general description and the following detailed description are exemplary and explanatory only and are not restrictive. Unless defined otherwise, technical and scientific terms used herein have the same meaning as commonly understood by one of ordinary skill in the art to which this invention belongs. See, for example Singleton et al., Dictionary of Microbiology and Molecular Biology 2nd ed., J. Wiley & Sons (New York, N.Y. 1994); Sambrook et al., Molecular Cloning, A Laboratory Manual, Cold Springs Harbor Press (Cold Springs Harbor, N.Y. 1989). It is to be understood that both the general description and the detailed description are exemplary and explanatory only and are not restrictive of the invention as claimed. In this application, the use of the singular includes the plural unless specifically stated otherwise. In this application, the use of “or” means “and/or” unless stated otherwise. Furthermore, the use of the term “including”, as well as other forms, such as “includes” and “included”, is not limiting. Also, terms such as “element” or “component” encompass both elements and components comprising one unit and elements and components that comprise more than one subunit unless specifically stated otherwise. Also, the use of the term “portion” can include part of a moiety or the entire moiety.

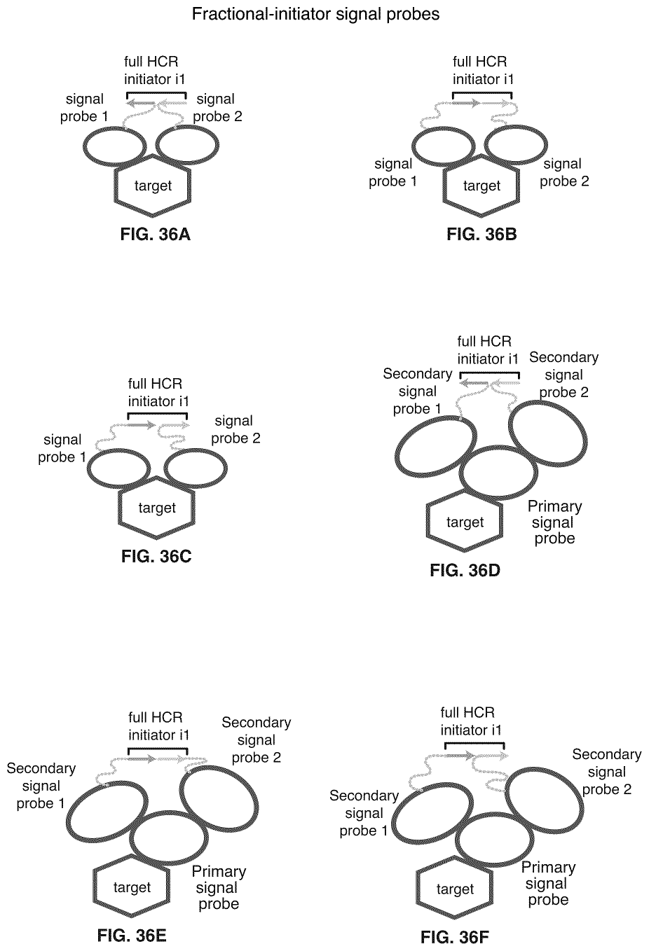

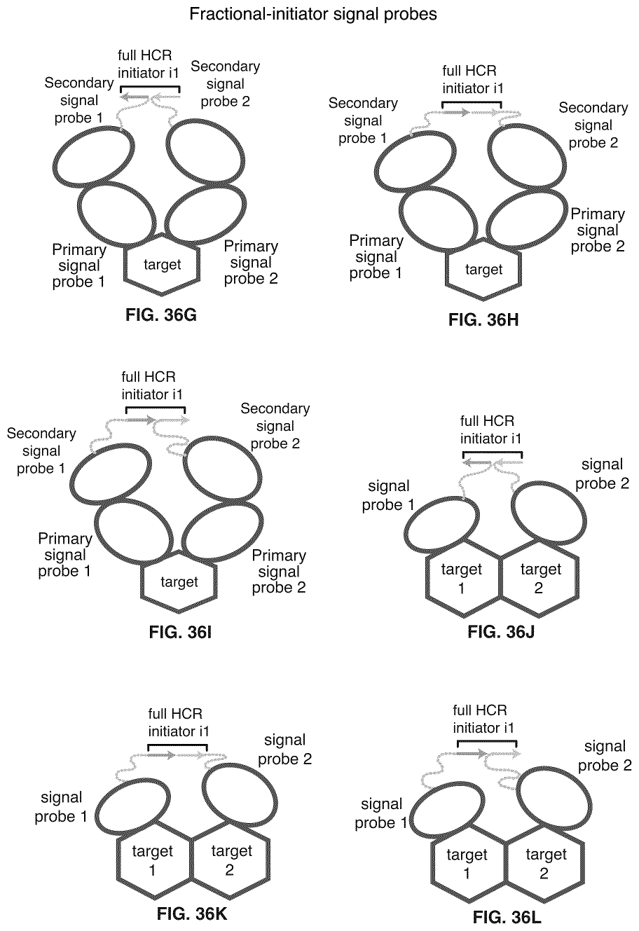

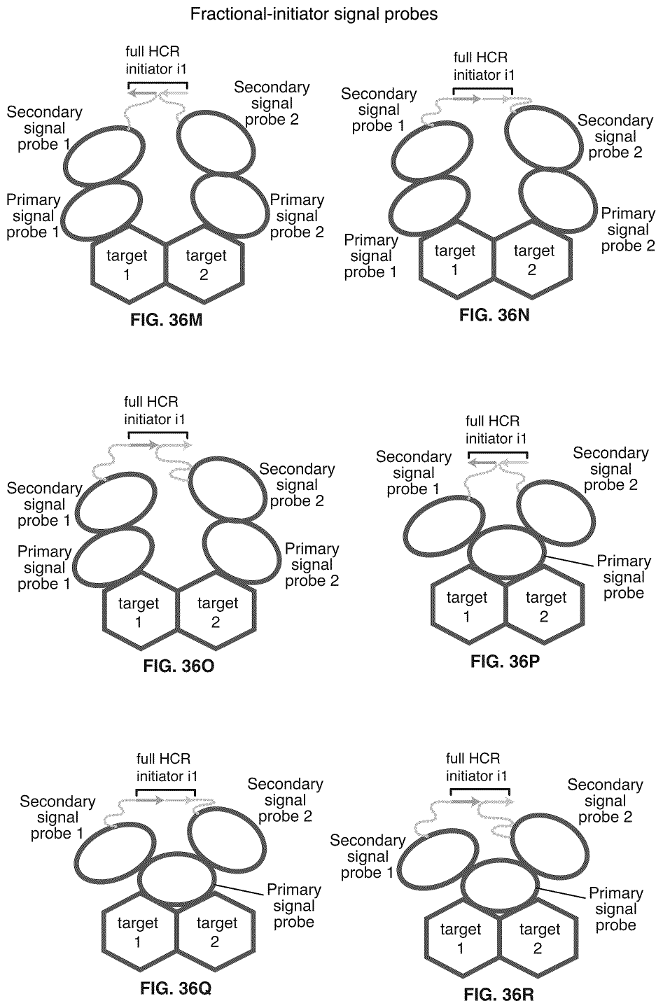

In some embodiments, any one or more of the optional elements of any one or more of the figures herein can be combined with any one or more of the other optional elements of any one or more of the figures herein. In some embodiments, any one or more of the compositions or steps provided in any of the figures provided herein can be combined with any of the other compositions or steps provided herein. As used herein, a generic reference to a set of figures (for example, FIG. 36) denotes all of the different figures contained within that number (for example, FIGS. 36A-36R), each combined together, one or more of them, or each in the alternative, unless otherwise denoted.

### REFERENCES

(1) Söderberg, O.; Gullberg, M.; Jarvius, M.; Ridderstrale, K.; Leuchowius, K. J.; Jarvius, J.; Wester, K.; Hydbring, P.; Bahram, F.; Larsson, L. G.; Landegren, U. Direct Observation of Individual Endogenous Protein Complexes in Situ by Proximity Ligation. Nat. Methods 2006, 3 (12), 995-1000.
(2) Weibrecht, I.; Gavrilovic, M.; Lindbom, L.; Landegren, U.; Wahlby, C.; Söderberg, O. Visualising Individual Sequence-Specific Protein-DNA Interactions in Situ. New Biotechnol. 2012, 29 (5), 589-598.
(3) Jung, J.; Lifland, A. W.; Zurla, C.; Alonas, E. J.; Santangelo, P. J. Quantifying RNA-Protein Interactions in Situ Using Modified-MTRIPs and Proximity Ligation. Nucleic Acids Res 2013, 41 (1), e12.
(4) Gomez, D.; Shankman, L. S.; Nguyen, A. T.; Owens, G. K. Detection of Histone Modifications at Specific Gene Loci in Single Cells in Histological Sections. Nat Methods 2013, 10 (2), 171-177.
(5) Leuchowius, K. J.; Clausson, C. M.; Grannas, K.; Erbilgin, Y.; Botling, J.; Zieba, A.; Landegren, U.; Söderberg, O. Parallel Visualization of Multiple Protein Complexes in Individual Cells in Tumor Tissue. Mol. Cell. Proteomics 2013, 12 (6), 1563-1571.
(6) Bellucci, A.; Fiorentini, C.; Zaltieri, M.; Missale, C.; Spano, P. The in Situ Proximity Ligation Assay to Probe Protein-Protein Interactions in Intact Tissues. Methods Mol Biol 2014, 1174, 397-405.
(7) Koos, B.; Andersson, L.; Clausson, C. M.; Grannas, K.; Klaesson, A.; Cane, G.; Söderberg, O. Analysis of Protein Interactions in Situ by Proximity Ligation Assays. Curr. Top. Microbiol. Immunol. 2013, 377, 111-126.
(8) Roussis, I. M.; Guille, M.; Myers, F. A.; Scarlett, G. P. RNA Whole-Mount in Situ Hybridisation Proximity Ligation Assay (rISH-PLA), an Assay for Detecting RNA-Protein Complexes in Intact Cells. PLoS One 2016, 11 (1), e0147967.
(9) Petruk, S.; Fenstermaker, T. K.; Black, K. L.; Brock, H. W.; Mazo, A. Detection of RNA-DNA Association by a Proximity Ligation-Based Method. Sci Rep 2016, 6, 27313.
(10) Black, K. L.; Petruk, S.; Fenstermaker, T. K.; Hodgson, J. W.; Caplan, J. L.; Brock, H. W.; Mazo, A. Chromatin Proteins and RNA Are Associated with DNA during All Phases of Mitosis. Cell Discov 2016, 2, 16038.
(11) Zurla, C.; Jung, J.; Blanchard, E. L.; Santangelo, P. J. A Novel Method to Quantify RNA-Protein Interactions in Situ Using FMTRIP and Proximity Ligation. Methods Mol Biol 2017, 1468, 155-170.
(12) Klaesson, A.; Grannas, K.; Ebai, T.; Heldin, J.; Koos, B.; Leino, M.; Raykova, D.; Oelrich, J.; Arngirden, L.; Söderberg, O.; Landegren, U. Improved Efficiency of in Situ Protein Analysis by Proximity Ligation Using UnFold Probes. Sci. Rep. 2018, 8 (1), 5400.
(13) Koos, B.; Cane, G.; Grannas, K.; Löf, L.; Arngirden, L.; Heldin, J.; Clausson, C. M.; Klaesson, A.; Hirvonen, M. K.; de Oliveira, F. M. S.; Talibov, V. O.; Pham, N. T.; Auer, M.; Danielson, U. H.; Haybaeck, J.; Kamali-Moghaddam, M.; Söderberg, O. Proximity-Dependent Initiation of Hybridization Chain Reaction. Nat. Commun. 2015, 6, 7294.
(14) Dirks, R. M.; Pierce, N. A. Triggered Amplification by Hybridization Chain Reaction. Proc. Natl. Acad. Sci. U.S.A. 2004, 101 (43), 15275-15278.
(15) Choi, H. M. T.; Chang, J. Y.; Trinh, L. A.; Padilla, J. E.; Fraser, S. E.; Pierce, N. A. Programmable in Situ Amplification for Multiplexed Imaging of mRNA Expression. Nat. Biotechnol. 2010, 28 (11), 1208-1212.
(16) Choi, H. M. T.; Beck, V. A.; Pierce, N. A. Next-Generation in Situ Hybridization Chain Reaction: Higher Gain, Lower Cost, Greater Durability. ACS Nano 2014, 8 (5), 4284-4294.
(17) Choi, H. M. T.; Schwarzkopf, M.; Fornace, M. E.; Acharya, A.; Artavanis, G.; Stegmaier, J.; Cunha, A.; Pierce, N. A. Third-Generation in Situ Hybridization Chain Reaction: Multiplexed, Quantitative, Sensitive, Versatile, Robust. Development 2018, 145, dev165753.
(18) Schwarzkopf, M.; Liu, M. C.; Schulte, S. J.; Ives, R.; Husain, N.; Choi, H. M. T.; Pierce, N. A. Hybridization Chain Reaction Enables a Unified Approach to Multiplexed, Quantitative, High-Resolution Immunohistochemistry and in Situ Hybridization. Development 2021, 148 (22), dev199847.
(19) Choi, H. M. T.; Calvert, C. R.; Husain, N.; Huss, D.; Barsi, J. C.; Deverman, B. E.; Hunter, R. C.; Kato, M.; Lee, S. M.; Abelin, A. C. T.; Rosenthal, A. Z.; Akbari, O. S.; Li, Y.; Hay, B. A.; Sternberg, P. W.; Patterson, P. H.; Davidson, E. H.; Mazmanian, S. K.; Prober, D. A.; van de Rijn, M.; Leadbetter, J. R.; Newman, D. K.; Readhead, C.; Bronner, M. E.; Wold, B.; Lansford, R.; Sauka-Spengler, T.; Fraser, S. E.; Pierce, N. A. Mapping a Multiplexed Zoo of mRNA Expression. Development 2016, 143, 3632-3637.
(20) Leino, M.; Heldin, J.; Sander, M. R.; Kermpatsou, D.; Raykova, D.; Koos, B.; Söderberg, O. Optimization of Proximity-Dependent Initiation of Hybridization Chain Reaction for Improved Performance. Mol. Syst. Des. Eng. 2019, 4 (5), 1058-1065.
(21) Burke, K. S.; Antilla, K. A.; Tirrell, D. A. A Fluorescence in Situ Hybridization Method to Quantify mRNA Translation by Visualizing Ribosome-mRNA Interactions in Single Cells. ACS Cent Sci 2017, 3 (5), 425-433.
(22) Pavani, S. R. P.; Thompson, M. A.; Biteen, J. S.; Moerner, W. E. Three-Dimensional, Single-Molecule Fluorescence Imaging beyond the Diffraction Limit by Using a Double-Helix Point Spread Function. Proc. Natl. Acad. Sci. U.S.A. 2009, 106 (9), 2995-2999.
(23) Shtengel, G.; Galbraith, J. A.; Galbraith, C. G.; Hess, H. F. Interferometric Fluorescent Super-Resolution Microscopy Resolves 3D Cellular Ultrastructure. Proc. Natl. Acad. Sci. U.S.A. 2009, 106 (9), 3125-3130.
(24) Qian, X.; Jin, L.; Lloyd, R. V. In Situ Hybridization: Basic Approaches and Recent Development. J. Histotechnol. 2004, 27 (1), 53-67.
(25) Silverman, A.; Kool, E. Oligonucleotide Probes for RNA-Targeted Fluorescence in Situ Hybridization. Adv. Clin. Chem. 2007, 43, 79-115.
(26) Ramos-Vara, J. A.; Miller, M. A. When Tissue Antigens and Antibodies Get along: Revisiting the Technical Aspects of Immunohistochemistry—the Red, Brown, and Blue Technique. Vet. Pathol. 2014, 51 (1), 42-87.
(27) Collins, M. L.; Irvine, B.; Tyner, D.; Fine, E.; Zayati, C.; Chang, C. A.; Horn, T.; Ahle, D.; Detmer, J.; Shen, L.-P.; Kolberg, J.; Bushnell, S.; Urdea, M. S.; Ho, D. D. A Branched DNA Signal Amplification Assay for Quantification of Nucleic Acid Targets below 100 Molecules/Ml. Nucleic Acids Res. 1997, 25 (15), 2979-2984.
(28) Bushnell, S.; Budde, J.; Catino, T.; Cole, J.; Derti, A.; Kelso, R.; Collins, M. L.; Molino, G.; Sheridan, P.; Monahan, J.; Urdea, M. ProbeDesigner: For the Design of Probesets for Branched DNA (bDNA) Signal Amplification Assays. Bioinformatics 1999, 15 (5), 348-355.
(29) Player, A. N.; Shen, L.-P.; Kenny, D.; Antao, V. P.; Kolberg, J. A. Single-Copy Gene Detection Using Branched DNA (bDNA) in Situ Hybridization. J. Histochem. Cytochem. 2001, 49 (5), 603-611.
(30) Qian, X.; Lloyd, R. V. Recent Developments in Signal Amplification Methods for in Situ Hybridization. Diagn. Mol. Pathol. 2003, 12 (1), 1-13.
(31) Wang, F.; Flanagan, J.; Su, N.; Wang, L.-C.; Bui, S.; Nielson, A.; Wu, X. Y.; Vo, H.-T.; Ma, X.-J.; Luo, Y. L. RNAscope: A Novel in Situ RNA Analysis Platform for Formalin-Fixed, Paraffin-Embedded Tissues. J. Mol. Diagn. 2012, 14 (1), 22-29.
(32) Kishi, J. Y.; Lapan, S. W.; Beliveau, B. J.; West, E. R.; Zhu, A.; Sasaki, H. M.; Saka, S. K.; Wang, Y.; Cepko, C. L.; Yin, P. SABER Amplifies FISH: Enhanced Multiplexed Imaging of RNA and DNA in Cells and Tissues. Nat Methods 2019, 16 (6), 533-544.
(33) Saka, S. K.; Wang, Y.; Kishi, J. Y.; Zhu, A.; Zeng, Y.; Xie, W.; Kirli, K.; Yapp, C.; Cicconet, M.; Beliveau, B. J.; Lapan, S. W.; Yin, S.; Lin, M.; Boyden, E. S.; Kaeser, P. S.; Pihan, G.; Church, G. M.; Yin, P. Immuno-SABER Enables Highly Multiplexed and Amplified Protein Imaging in Tissues. Nat. Biotechnol. 2019, 37 (9), 1080-1090.
(34) Zhou, Y.; Calciano, M.; Hamann, S.; Leamon, J. H.; Strugnell, T.; Christian, M. W.; Lizardi, P. M. In Situ Detection of Messenger RNA Using Digoxigenin-Labeled Oligonucleotides and Rolling Circle Amplification. Exp. Mol. Pathol. 2001, 70, 281-288.
(35) Schweitzer, B.; Kingsmore, S. Combining Nucleic Acid Amplification and Detection. Curr. Opin. Biotechnol. 2001, 12, 21-27.
(36) Larsson, C.; Koch, J.; Nygren, A.; Janssen, G.; Raap, A. K.; Landegren, U.; Nilsson, M. In Situ Genotyping Individual DNA Molecules by Target-Primed Rolling-Circle Amplification of Padlock Probes. Nat. Methods 2004, 1 (3), 227-232.
(37) Zhou, H.; Bouwman, K.; Schotanus, M.; Verweij, C.; Marrero, J. A.; Dillon, D.; Costa, J.; Lizardi, P.; Haab, B. B. Two-Color, Rolling-Circle Amplification on Antibody Microarrays for Sensitive, Multiplexed Serum-Protein Measurements. Genome Biol. 2004, 5 (4), R28.
(38) Larsson, C.; Grundberg, I.; Söderberg, O.; Nilsson, M. In Situ Detection and Genotyping of Individual mRNA Molecules. Nat. Methods, 2010, 7, 395-397.
(39) Tautz, D.; Pfeifle, C. A Non-Radioactive in Situ Hybridization Method for the Localization of Specific RNAs in Drosophila Embryos Reveals Translational Control of the Segmentation Gene Hunchback. Chromosoma 1989, 98, 81-85.
(40) Harland, R. M. In Situ Hybridization: An Improved Whole-Mount Method for Xenopus Embryos. Methods Cell Biol. 1991, 36, 685-695.
(41) Lehmann, R.; Tautz, D. In Situ Hybridization to RNA. In Drosophila melanogaster: practical uses in cell and molecular biology; Goldstein, L. S. B., Fyrberg, E. A., Eds.; Methods in Cell Biology; Elsevier Academic Press: San Diego, C A, 1994; Vol. 44, pp 575-598.
(42) Kerstens, H. M. J.; Poddighe, P. J.; Hanselaar, A. G. J. M. A Novel In-Situ Hybridization Signal Amplification Method Based on the Deposition of Biotinylated Tyramine. J. Histochem. Cytochem. 1995, 43 (4), 347-352.
(43) Nieto, M. A.; Patel, K.; Wilkinson, D. G. In Situ Hybridization Analysis of Chick Embryos in Whole Mount and Tissue Sections. In Methods in Avian Embryology; Bronner-Fraser, M., Ed.; Methods in Cell Biology; Elsevier Academic Press: San Diego, C A, 1996; Vol. 51, pp 219-235.
(44) Pernthaler, A.; Pernthaler, J.; Amann, R. Fluorescence in Situ Hybridization and Catalyzed Reporter Deposition for the Identification of Marine Bacteria. Appl. Environ. Microbiol. 2002, 68 (6), 3094-3101. https://doi.org/10.1128/Aem.68.6.3094-3101.2002.
(45) Kosman, D.; Mizutani, C. M.; Lemons, D.; Cox, W. G.; McGinnis, W.; Bier, E. Multiplex Detection of RNA Expression in Drosophila Embryos. Science 2004, 305, 846.
(46) Thisse, B.; Heyer, V.; Lux, A.; Alunni, V.; Degrave, A.; Seiliez, I.; Kirchner, J.; Parkhill, J. P.; Thisse, C. Spatial and Temporal Expression of the Zebrafish Genome by Large-Scale in Situ Hybridization Screening. In The Zebrafish: 2nd Edition Genetics Genomics and Informatics; Detrich III, H. W. D., Zon, L. I., Westerfield, M., Eds.; Methods in Cell Biology; Elsevier Academic Press: San Diego, C A, 2004; Vol. 77, pp 505-519.
(47) Denkers, N.; Garcia-Villalba, P.; Rodesch, C. K.; Nielson, K. R.; Mauch, T. J. FISHing for Chick Genes: Triple-label Whole-mount Fluorescence in Situ Hybridization Detects Simultaneous and Overlapping Gene Expression in Avian Embryos. Dev. Dyn. 2004, 229 (3), 651-657.
(48) Clay, H.; Ramakrishnan, L. Multiplex Fluorescent in Situ Hybridization in Zebrafish Embryos Using Tyramide Signal Amplification. Zebrafish 2005, 2 (2), 105-111.
(49) Barroso-Chinea, P.; Aymerich, M. S.; Castle, M. M.; Perez-Manso, M.; Tunon, T.; Erro, E.; Lanciego, J. L. Detection of Two Different mRNAs in a Single Section by Dual in Situ Hybridization: A Comparison between Colorimetric and Fluorescent Detection. J. Neurosci. Methods 2007, 162 (1-2), 119-128.
(50) Acloque, H.; Wilkinson, D. G.; Nieto, M. A. In Situ Hybridization Analysis of Chick Embryos in Whole-Mount and Tissue Sections. In Avian Embryology, 2nd Edition; Bronner-Fraser, M., Ed.; Methods in Cell Biology; Elsevier Academic Press: San Diego, C A, 2008; Vol. 87, pp 169-185.
(51) Piette, D.; Hendrickx, M.; Willems, E.; Kemp, C. R.; Leyns, L. An Optimized Procedure for Whole-Mount in Situ Hybridization on Mouse Embryos and Embryoid Bodies. Nat. Protoc. 2008, 3 (7), 1194-1201.
(52) Thisse, C.; Thisse, B. High-Resolution in Situ Hybridization to Whole-Mount Zebrafish Embryos. Nat. Protoc. 2008, 3 (1), 59-69.
(53) Weiszmann, R.; Hammonds, A. S.; Celniker, S. E. Determination of Gene Expression Patterns Using High-Throughput RNA in Situ Hybridization to Whole-Mount Drosophila Embryos. Nat. Protoc. 2009, 4 (5), 605-618.
(54) Takakura, N.; Yoshida, H.; Ogura, Y.; Kataoka, H.; Nishikawa, S.; Nishikawa, S. PDGFR Alpha Expression during Mouse Embryogenesis: Immunolocalization Analyzed by Whole-Mount Immunohistostaining Using the Monoclonal Anti-Mouse PDGFR Alpha Antibody APA5. J. Histochem. Cytochem. 1997, 45 (6), 883-893.
(55) Sillitoe, R. V.; Hawkes, R. Whole-Mount Immunohistochemistry: A High-Throughput Screen for Patterning Defects in the Mouse Cerebellum. J. Histochem. Cytochem. 2002, 50 (2), 235-244.
(56) Ahnfelt-Ronne, J.; Jorgensen, M. C.; Hald, J.; Madsen, O. D.; Serup, P.; Hecksher-Sorensen, J. An Improved Method for Three-Dimensional Reconstruction of Protein Expression Patterns in Intact Mouse and Chicken Embryos and Organs. J. Histochem. Cytochem. 2007, 55 (9), 925-930.
(57) Psychoyos, D.; Finnell, R. Method for Whole Mount Antibody Staining in Chick. J. Vis. Exp. 2009, No. 24, e956.
(58) Fujisawa, S.; Yarilin, D.; Fan, N.; Turkekul, M.; Xu, K.; Barlas, A.; Manova-Todorova, K. Understanding the Three-Dimensional World from Two-Dimensional Immunofluorescent Adjacent Sections. J. Pathol. Inform. 2015, 6, 27.
(59) Staudt, N.; Muller-Sienerth, N.; Fane-Dremucheva, A.; Yusaf, S. P.; Millrine, D.; Wright, G. J. A Panel of Recombinant Monoclonal Antibodies against Zebrafish Neural Receptors and Secreted Proteins Suitable for Wholemount Immunostaining. Biochem. Biophys. Res. Commun. 2015, 456 (1), 527-533.
(60) Wiedorn, K. H.; Kuhl, H.; Galle, J.; Caselitz, J.; Vollmer, E. Comparison of In-Situ Hybridization, Direct and Indirect in-Situ PCR as Well as Tyramide Signal Amplification for the Detection of HPV. Histochem. Cell Biol. 1999, 111, 89-95.
(61) Söderberg, O.; Gullberg, M.; Jarvius, M.; Ridderstrale, K.; Leuchowius, K. J.; Jarvius, J.; Wester, K.; Hydbring, P.; Bahram, F.; Larsson, L. G.; Landegren, U. Direct Observation of Individual Endogenous Protein Complexes in Situ by Proximity Ligation. Nat Methods, 2006, 3, 995-1000.
(62) Koos, B.; Andersson, L.; Clausson, C. M.; Grannas, K.; Klaesson, A.; Cane, G.; Söderberg, O. Analysis of Protein Interactions in Situ by Proximity Ligation Assays. Curr Top Microbiol Immunol, 2014, 377, 111-126.
(63) Chitaev, N. A.; Troyanovsky, S. M. Adhesive but Not Lateral E-Cadherin Complexes Require Calcium and Catenins for Their Formation. J. Cell Biol. 1998, 142 (3), 837-846.
(64) Wahl, J. K.; Kim, Y. J.; Cullen, J. M.; Johnson, K. R.; Wheelock, M. J. N-Cadherin-Catenin Complexes Form Prior to Cleavage of the Proregion and Transport to the Plasma Membrane. J. Biol. Chem. 2003, 278 (19), 17269-17276.
(65) Yano, H.; Mazaki, Y.; Kurokawa, K.; Hanks, S. K.; Matsuda, M.; Sabe, H. Roles Played by a Subset of Integrin Signaling Molecules in Cadherin-Based Cell-Cell Adhesion. J. Cell Biol. 2004, 166 (2), 283-295.
(66) Berx, G.; Roy, F. V. The E-Cadherin/Catenin Complex: An Important Gatekeeper in Breast Cancer Tumorigenesis and Malignant Progression. Breast Cancer Res. 2001, 3 (5), 289.
(67) Karayiannakis, A. J.; Nakopoulou, L.; Gakiopoulou, H.; Keramopoulos, A.; Davaris, P. S.; Pignatelli, M. Expression Patterns of β-Catenin in in Situ and Invasive Breast Cancer. Eur. J Surg. Oncol. EJSO 2001, 27 (1), 31-36.

What is claimed is:

1. A composition comprising:
a. a first fractional-initiator probe comprising:
i. a first target-binding domain configured to bind directly or indirectly to a first target,
ii. a first proximity domain,
iii. a first fractional initiator,
b. a second fractional-initiator probe comprising:
i. a second target-binding domain configured to bind directly or indirectly to a second target,
ii. a second proximity domain,
iii. a second fractional initiator,
c. a proximity probe configured to bind the first proximity domain and the second proximity domain;
d. a hybridization chain reaction (HCR) amplifier comprising two or more HCR hairpin monomers at least one of which comprises a reporter; and
wherein when the first fractional-initiator probe is bound to the first target and the second fractional-initiator probe is bound to the second target; and the first and the second target are bound to each other and/or are proximal, the proximity probe is able to bind the first proximity domain and the second proximity domain to colocalize a full initiator comprising the first fractional initiator and the second fractional initiator, and wherein the colocalized full initiator is configured to initiate HCR signal amplification whereupon the HCR hairpin monomers self-assemble into a tethered HCR amplification polymer thereby generating a signal.

2. The composition of claim 1, wherein at least one of the HCR hairpin monomers comprises an input domain and wherein the first fractional initiator and second fractional initiator together form a full initiator configured to hybridize to the input domain.

3. The composition of claim 1, further comprising:
a. a third fractional-initiator probe comprising:
i. a third target-binding domain configured to bind directly or indirectly to a third target,
ii. a third proximity domain,
iii. a third fractional initiator; and
b. a proximity probe further configured to bind the third proximity domain.

4. The composition of claim 1, wherein the first target is a protein, a nucleic acid, or a combination thereof, and wherein the second target is a protein, a nucleic acid, or a combination thereof.

5. The composition of claim 4, wherein the first target and the second target are bound to each other.

6. The composition of claim 4, wherein the first target and the second target are the same molecule.

7. The composition of claim 1, wherein the proximity probe contains one or more clamp domains configured to bind to some or all of the first fractional initiator and/or the second fractional initiator if one target is not proximal.

8. The composition of claim 1, where the first fractional-initiator probe and/or the second fractional-initiator probe comprises an antibody, a nanobody, and/or an oligonucleotide.

9. The composition of claim 1, wherein the colocalized full initiator is used to mediate generation of a signal directly or indirectly.

10. The composition of claim 1, wherein the colocalized full initiator triggers self-assembly of metastable fluorophore-labeled HCR hairpins into a tethered fluorescent amplification polymer to generate an amplified signal at the site of the targets.

11. The composition of claim 1, wherein the first target and second target are selected from protein, RNA, DNA, or other molecules.

12. The composition of claim 1, wherein the first target and second target form a complex.

13. The composition of claim 12, wherein the composition comprises additional fractional initiators for each of N target complexes to be detected in a sample.

14. The composition of claim 13, wherein the N target complexes are each detected in the same sample using a different pair of fractional-initiator probes, proximity probe, and HCR amplifier for each target complex.

15. The composition of claim 1, wherein the sequence of the first fractional initiator is selected from SEQ ID NO: 1, SEQ ID NO: 4, and SEQ ID NO: 7; the sequence of the second fractional initiator is selected from SEQ ID NO: 2, SEQ ID NO: 5, and SEQ ID NO: 8; and the sequence of the proximity probe is selected from SEQ ID NO: 3, SEQ ID NO: 6, and SEQ ID NO: 9.

16. The composition of claim 1, wherein the at least one reporter comprises a fluorophore, a chromophore, a luminophore, a phosphor, a FRET pair, a member of a FRET pair, a quencher, a fluorophore/quencher pair, a rare earth element or compound, a radioactive molecule, a nucleotide, an amino acid, an oligonucleotide, DNA, RNA, 2′Ome-RNA, a chemically modified nucleic acid, a synthetic nucleic acid analog, a chemically modified protein, a synthetic protein analog, a peptide, a binding substrate, a carbon atom, a chemical linker, a magnetic molecule, carbon black (CB), carbon nanotubes, magnetized carbon nanotubes, gold nanoparticles (AuNP), gold nanoshells, gold nanorods, silver-shelled gold nanoparticles, latex, magnetic nanoparticles, silica nanoparticles, fluorophore-loaded nanoparticles, dye-loaded nanoparticles, an enzyme, or any combination thereof.

17. The composition of claim 1, wherein the at least one reporter mediates generation of the signal directly or indirectly.

18. The composition of claim 1, wherein the at least one reporter comprises a hapten, a ligand, an oligonucleotide, digoxigenin (DIG), fluorescein isothiocyanate (FITC), a fluorophore, biotin, dinitrophenol, aniline, or an enzyme utilized to facilitate generation of the signal.

19. The composition of claim 1, wherein the HCR amplification polymer mediates catalytic reporter deposition (CARD).

20. A method comprising:
a. Providing:
i. A sample containing a first target and a second target;
ii. A first fractional-initiator probe comprising:
1. a first target-binding domain configured to bind directly or indirectly to the first target,
2. a first proximity domain,
3. a first fractional initiator;
iii. A second fractional-initiator probe comprising:
1. a second target-binding domain configured to bind directly or indirectly to the second target,
2. a second proximity domain,
3. a second fractional initiator;
iv. A proximity probe configured to bind the first proximity domain and the second proximity domain;
v. A hybridization chain reaction (HCR) amplifier comprising two or more HCR hairpin monomers at least one of which comprises a reporter;
b. adding the first fractional-initiator probe and the second fractional-initiator probe to the sample;
c. adding the proximity probe to the sample;
d. adding the HCR amplifier to the sample;
e. detecting a signal indicating the presence of an HCR polymer;
wherein when the first fractional-initiator probe is bound to the first target and the second fractional-initiator probe is bound to the second target, and when the first and second target are bound to each other or are proximal, then the proximity probe binds to the first proximity domain and the second proximity domain to colocalize a full initiator; and
wherein the colocalized full initiator initiates polymerization of the HCR monomers thereby generating a signal.

21. The method of claim 20, wherein the method further comprises providing, a third fractional-initiator probe comprising:
1. a third target-binding domain configured to bind directly or indirectly to the third target,
2. a third proximity domain, and
3. a third fractional initiator.

22. The method of claim 21, wherein the proximity probe is configured to bind the first proximity domain the second proximity domain, and the third proximity domain.

23. The method of claim 20, wherein a wash step is performed to remove unbound fractional-initiator probes following b and before c.

24. The method of claim 20, wherein a wash step is performed to remove unbound proximity probes following c and before d.

25. The method of claim 20, wherein a wash step is performed to remove unbound HCR hairpins following d.

26. The method of claim 20, wherein the signal is removed following e.

27. The method of claim 26, wherein any of the above steps are repeated to detect a signal for the same or different targets.

28. The method of claim 20, wherein the signal is detected by a fluorescence microscope, a fluorescence scanner, a camera, a mobile phone camera, a mass spectrometer, a mass spectrometry microscope, or a radioactive scanner.

29. The method of claim 20, further comprising providing a helper probe to maximize signal generation.

30. The method of claim 20, wherein the first target is a protein, a nucleic acid, or a combination thereof, and wherein the second target is a protein, a nucleic acid, or a combination thereof.

31. The method of claim 30, wherein the first target and the second target are bound to each other.

32. The method of claim 30, wherein the first target and the second target are the same molecule.

33. The method of claim 20, wherein the proximity probe contains one or more clamp domains configured to bind to some or all of the first fractional initiator and/or the second fractional initiator if one target is not proximal.

34. The method of claim 20, where the first fractional-initiator probe and/or the second fractional-initiator probe comprises a target binding region comprising an antibody, a nanobody, and/or an oligonucleotide.

35. The method of claim 20, wherein the colocalized full initiator is used to mediate generation of a signal directly or indirectly.

36. The method of claim 20, wherein the colocalized full initiator triggers self-assembly of metastable fluorophore-labeled HCR hairpins into a tethered fluorescent amplification polymer to generate an amplified signal at the site of the proximal targets.

37. The method of claim 20, wherein the colocalized full initiator is utilized to detect one or more protein:protein complexes, RNA:protein complexes, RNA:RNA complexes, DNA:protein complexes, DNA:protein:protein complexes, or a complex of three or more RNA, DNA, and/or protein or other molecules in a sample.

38. The method of claim 20, wherein fractional-initiator probes that bind nonspecifically do not colocalize a full initiator and do not initiate HCR.

39. The method of claim 20, wherein N target complexes are each detected in the same sample using a different pair of fractional-initiator probes, proximity probe, and HCR amplifier for each target complex.

40. The method of claim 20, wherein the sequence of the first fractional initiator is selected from SEQ ID NO: 1, SEQ ID NO: 4, and SEQ ID NO: 7; the sequence of the second fractional initiator is selected from SEQ ID NO: 2, SEQ ID NO: 5, and SEQ ID NO: 8; and the sequence of the proximity probe is selected from SEQ ID NO: 3, SEQ ID NO: 6, and SEQ ID NO: 9.

41. The method of claim 20, wherein the at least one reporter comprises a fluorophore, a chromophore, a luminophore, a phosphor, a FRET pair, a member of a FRET pair, a quencher, a fluorophore/quencher pair, a rare earth element or compound, a radioactive molecule, a nucleotide, an amino acid, an oligonucleotide, DNA, RNA, 2′Ome-RNA, a chemically modified nucleic acid, a synthetic nucleic acid analog, a chemically modified protein, a synthetic protein analog, a peptide, a binding substrate, a carbon atom, a chemical linker, a magnetic molecule, carbon black (CB), carbon nanotubes, magnetized carbon nanotubes, gold nanoparticles (AuNP), gold nanoshells, gold nanorods, silver-shelled gold nanoparticles, latex, magnetic nanoparticles, silica nanoparticles, a fluorophore, fluorophore-loaded nanoparticles, dye-loaded nanoparticles, an enzyme, any combination thereof.

42. The method of claim 20, wherein the at least one reporter mediates generation of the signal directly or indirectly.

43. The method of claim 20, wherein the at least one reporter comprises a hapten, a ligand, an oligonucleotide, digoxigenin (DIG), fluorescein isothiocyanate (FITC), a fluorophore, biotin, dinitrophenol, aniline, or an enzyme utilized to facilitate generation of the signal.

44. The method of claim 20, wherein the HCR amplification polymer mediates catalytic reporter deposition (CARD).


SEQUENCE LISTING (9 sequences)

SEQ ID NO: 1 - DNA, 49 bp, synthetic construct
  source 1..49: mol_type = other DNA
gaggagggcagcaaacggtcgcccatgtgtacccgaaattcaagtcagc

SEQ ID NO: 2 - DNA, 49 bp, synthetic construct
  source 1..49: mol_type = other DNA
ttcctgattctattacttgcctgatccctatgaagagtcttcctttacg

SEQ ID NO: 3 - DNA, 50 bp, synthetic construct
  source 1..50: mol_type = other DNA
cttgaatttcgggtacacatgggctaagggatcaggcaagtaatagaatc

SEQ ID NO: 4 - DNA, 49 bp, synthetic construct
  source 1..49: mol_type = other DNA
gcaaactaaacatcccacaaggtgaaagctggtacgaataagactacgc

SEQ ID NO: 5 - DNA, 49 bp, synthetic construct
  source 1..49: mol_type = other DNA
ctgcaccggtatatgttctgaaggtgatgcatccaactctaactaaatc

SEQ ID NO: 6 - DNA, 50 bp, synthetic construct
  source 1..50: mol_type = other DNA
gtcttattcgtaccagctttcaccaccatcaccttcagaacatataccgg

SEQ ID NO: 7 - DNA, 49 bp, synthetic construct
  source 1..49: mol_type = other DNA
ctaacaatctaaacatactcgagggtgcggtctattctatttccaacgt

SEQ ID NO: 8 - DNA, 49 bp, synthetic construct
  source 1..49: mol_type = other DNA
tattgcgtgttaggtgagtttgagatttgtacacgcccaagaacataaa

SEQ ID NO: 9 - DNA, 50 bp, synthetic construct
  source 1..50: mol_type = other DNA
ggaaatagaatagaccgcaccctcaccaaatctcaaactcacctaacacg